(19) United States
(12) Patent Application Publication
(10) Pub. No.: US 2025/0085300 A1
(43) Pub. Date: Mar. 13, 2025

(54) STABILITY-ENHANCING COMPOSITIONS AND METHODS OF PREPARING COMPOUNDS

(71) Applicant: FOGHORN THERAPEUTICS INC., Cambridge, MA (US)

(72) Inventors: Liyue HUANG, Sharon, MA (US); David B. TERRY, Watertown, MA (US); Joshua D. WAETZIG, Waltham, MA (US); Chong-Hui GU, Waban, MA (US)

(21) Appl. No.: 18/292,508

(22) PCT Filed: Jul. 28, 2022
(86) PCT No.: PCT/US2022/038668

§ 371 Date: Jan. 26, 2024

Related U.S. Application Data
(60) Provisional application No. 63/226,623, filed on Jul. 28, 2021.

(51) Int. Cl. G01N 33/94; C07D 519/00; G01N 33/68

(52) U.S. Cl. CPC G01N 33/94; C07D 519/00; G01N 33/6851

(57) ABSTRACT

Chiral glutarimide stereochemical identity-preserving methods and compositions are disclosed. Also disclosed are methods of preparing chiral glutarimides stereoretentively.

### BACKGROUND

Thalidomide and its derivatives (e.g., lenalidomide, pomalidomide, and degraders based on them) have recently become of interest in the medical field, especially in oncology, as being useful for targeted protein degradation. It is believed that thalidomide and its derivatives mediate targeted protein degradation by targeting the protein slated for degradation to an E3 ubiquitin ligase (e.g., cereblon) for subsequent proteasomal degradation.

Thalidomide and its derivatives (e.g., lenalidomide, pomalidomide, and degraders based on them) are recognized for their stereochemical instability due to the propensity for epimerization of the stereogenic center at the α-carbon of the glutarimide moiety present in thalidomide and its derivatives. Such stereochemical instability impairs applications reliant upon the structural integrity of the compound of interest that includes the glutarimide moiety present in thalidomide and its derivatives. For example, biological studies, such as absorption-distribution-metabolism-excretion (ADME) and pharmacokinetic (PK) studies, often require quantification of specific compounds, and post-sampling epimerization of the stereogenic center at the α-carbon of the glutarimide moiety present in thalidomide and its derivatives may impact the accuracy and precisions of the biological study. There is a need for compositions and methods for enhancing the stereochemical integrity of thalidomide and its derivatives, especially in a body fluid after its sampling.

Moreover, preparation of compounds containing a stereogenic center at the α-carbon of the glutarimide moiety is often achieved through preparation of a racemate and subsequent isolation of the desired enantiomer through resolution, e.g., using costly methods, such as preparatory high performance liquid chromatography or supercritical fluid chromatography. In addition to being costly, such resolution methods are not atom-economical, as half of the starting material is converted to an undesired enantiomer during the racemate preparation. There is a need for new methods for atom-economical syntheses of stereochemically enriched compounds containing an epimerizable stereogenic center at the α-carbon of the glutarimide moiety.

### SUMMARY

In one aspect, the invention provides a method of determining the level of a chiral glutarimide including an epimerizable stereogenic center at the α-carbon of the glutarimide ring in a subject, the method including the steps of: collecting a body fluid from the subject into a container including a solution including citric acid to form a composition; and determining the level of the chiral glutarimide in the composition, thereby determining the level of a chiral glutarimide in a subject.

In another aspect, the invention provides a method of preparing a composition including a chiral glutarimide including an epimerizable stereogenic center at the α-carbon of the glutarimide ring and a body fluid, the method including the steps of collecting the body fluid from a subject into a container including a solution including citric acid, the body fluid including the chiral glutarimide.

In yet another aspect, the invention provides a composition including a chiral glutarimide including an epimerizable stereogenic center at the α-carbon of the glutarimide ring, a body fluid, and citrate buffer, where the composition is enriched for one of the stereoisomers of the epimerizable stereogenic center.

In some embodiments, the body fluid is blood. In some embodiments, the solution (e.g., aqueous solution) including citric acid is 0.5 M to 5 M. In some embodiments, the solution including citric acid is 3 M citric acid (e.g., 3 M aqueous citric acid)). In some embodiments, the ratio of body fluid to the solution of citric acid in the composition is 99 to 1. In some embodiments, citric acid is present in an amount providing 0.005 M to 0.05 M (e.g., 0.03 M) concentration of citric acid in the composition.

In still another aspect, the invention provides a method of determining the level of a chiral glutarimide including an epimerizable stereogenic center at the α-carbon of the glutarimide ring in a subject, the method including the steps of: collecting a body fluid from the subject; separating the body fluid into two or more components; combining one of the components and citric acid to form a composition; and determining the level of the chiral glutarimide in the composition, thereby determining the level of a chiral glutarimide in a subject.

In a further aspect, the invention provides a method of preparing a composition including a chiral glutarimide including an epimerizable stereogenic center at the α-carbon of the glutarimide ring and a component of a body fluid, the method including the step of combining citric acid and the component of the body fluid collected from a subject.

In yet further aspect, the invention provides a composition including a chiral glutarimide including an epimerizable stereogenic center at the α-carbon of the glutarimide ring, a component of a body fluid, and citrate buffer, where the composition is enriched for one of the stereoisomers of the epimerizable stereogenic center.

In some embodiments, the component of a body fluid is blood serum or blood plasma. In some embodiments, the component of a body fluid is blood serum. In some embodiments, the component of a body fluid is blood plasma. In some embodiments, the citric acid is in lyophilized form. In some embodiments, the ratio of the component of body fluid to citric acid in the composition is 99 to 1. In some embodiments, citric acid is present in an amount providing 0.005 M to 0.05 M (e.g., 0.03 M) concentration of citric acid in the composition. In some embodiments, the body fluid is stored at 0° C. to 4° C. between the step of collecting the body fluid and the step of preparing the component of the body fluid.

In some embodiments, the chiral glutarimide including an epimerizable stereogenic center at the α-carbon of the glutarimide ring is a compound of formula I:

A-L-B Formula I,
where
L is a linker;
B is a degradation moiety having the structure:

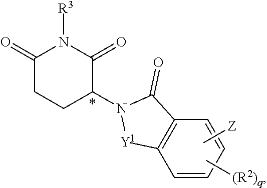

where
   *designates the stereoenriched epimerizable stereogenic center at the α-carbon of the glutarimide ring;
Y¹ is

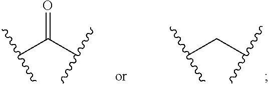

R³ is H, optionally substituted C₁-C₆ alkyl, or optionally substituted C₁-C₆ heteroalkyl;
q is 0, 1, 2, 3, or 4;
each R₂ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; and
Z is a substituent; and
A is a protein binding moiety,
or a pharmaceutically acceptable salt thereof.

In some embodiments, the protein binding moiety has the structure of Formula E-3, Formula E-4, Formula G-2, Formula G-3, or Formula E-5:

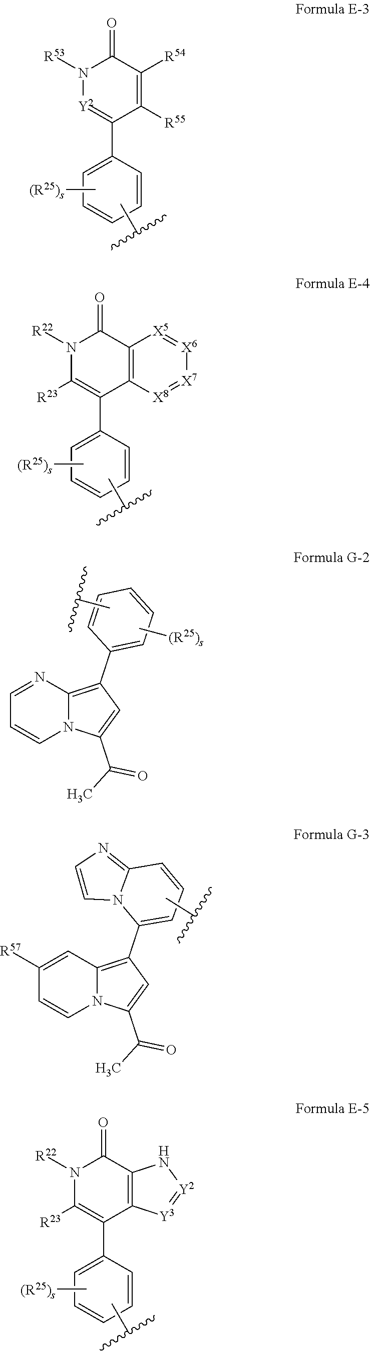

where
Y² is N or CR²³;
R²² is H, optionally substituted C₁-C₆ alkyl, or optionally substituted C₁-C₆ heteroalkyl;
R²³ is H, halogen, optionally substituted C₁-C₆ alkyl, or optionally substituted C₆-C₁₀ aryl;
s is 0, 1, 2, 3, or 4;
each R²⁵ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino;
R⁵³ is H, optionally substituted C₁-C₆ alkyl, optionally substituted C₁-C₆ heteroalkyl, or optionally substituted C₃-C₁₀ carbocyclyl;
R⁵⁴ is H or optionally substituted C₂-C₉ heteroaryl;
R⁵⁵ is H or N(R^{a})₂, where each R^{a} is independently H, optionally substituted C₁-C₆ alkyl, optionally substituted C₁-C₆ heteroalkyl, or optionally substituted C₃-C₁₀ carbocyclyl, or two geminal Ra, together with the nitrogen atom to which they are attached, combine to form optionally substituted C₂-C₉ heterocyclyl;
each of X⁵, X⁶, X⁷, and X⁸ is, independently, N or CR⁵⁶;
each R⁵⁶ is, independently, H or N(R^{a})₂, where Ra is H, optionally substituted C₁-C₆ alkyl, optionally substituted C₁-C₆ heteroalkyl, or optionally substituted C₃-C₁₀ carbocyclyl, or two geminal Ra, together with the nitrogen atom to which they are attached, combine to form optionally substituted C₂-C₉ heterocyclyl;
R⁵⁷ is optionally substituted C₂-C₁₀ heterocyclyl;
each of Y² and Y³ is, independently, N or CR⁵⁸; and
each R⁵⁸ is, independently, H or optionally substituted C₁-C₆ alkyl,
where if R⁵³ is H and R⁵⁴ is H, then R⁵⁵ is NR^{a}; if R⁵⁴ is H and R⁵⁵ is H, then R⁵³ is optionally substituted C₃-C₁₀ carbocyclyl; and if R⁵³ is H and R⁵⁵ is H, then R⁵⁴ is optionally substituted C₂-C₉ heteroaryl,
or a pharmaceutically acceptable salt thereof.
In some embodiments, A has the structure of Formula E-3. In some embodiments, A has the structure of Formula E-4. In some embodiments, A has the structure of Formula G-2. In some embodiments, A has the structure of Formula G-3. In some embodiments, A has the structure of Formula E-5. In some embodiments, s is 0, 1, or 2.

In some embodiments, the degradation moiety has the structure of Formula A-1:

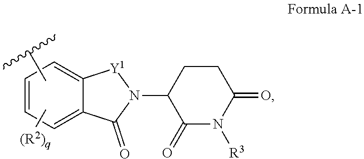

where
Y¹ is

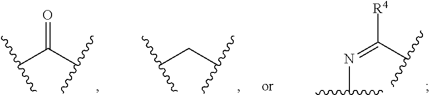

R³ and R⁴ are, independently, H, optionally substituted C₁-C₆ alkyl, or optionally substituted C₁-C₆ heteroalkyl;
q is 0, 1, 2, 3, or 4; and
each R₂ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino,
or a pharmaceutically acceptable salt thereof.

In some embodiments, R³ is H or optionally substituted C₁-C₆ alkyl. In some embodiments, R³ is H or CH₃. In some embodiments, R³ is H. In some embodiments, R³ is CH₃.

In some embodiments, Y¹ is

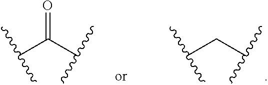

In some embodiments, Y¹ is

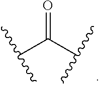 [SMILES: *C(*)=O]

In some embodiments, each R² is, independently, optionally substituted C₁-C₆ alkyl, optionally substituted C₁-C₆ heteroalkyl, hydroxyl, or optionally substituted amino.

In some embodiments, q is 0 or 1. In some embodiments, q is 0.

In some embodiments, the degradation moiety has the structure of Formula A-1a:

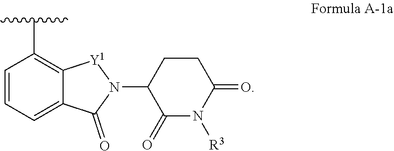

In some embodiments, the degradation moiety has the structure of Formula A-1b:

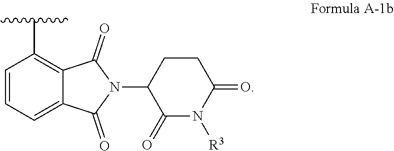

In some embodiments, the degradation moiety has the structure of Formula A-1c:

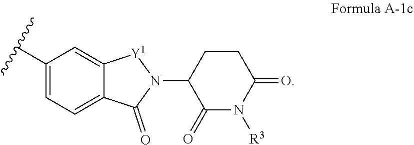

In some embodiments, the degradation moiety has the structure of Formula A-1d:

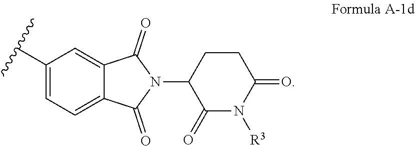

In some embodiments, the degradation moiety has the structure:

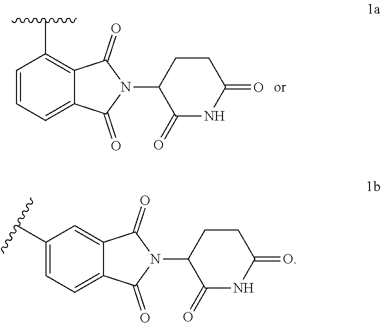 [SMILES: *c1cccc2c1C(=O)N(C1CCC(=O)NC1=O)C2=O ; "or" ; *c1ccc2c(c1)C(=O)N(C1CCC(=O)NC1=O)C2=O]

In some embodiments, the linker has the structure of Formula II:

A¹-(B¹)_{f}-(C¹)_{g}-(B²)ₕ-(D)-(B³)ᵢ-(C²)ⱼ-(B⁴)ₖ-A² Formula II
where
A¹ is a bond between the linker and A;
A² is a bond between B and the linker;
each of B¹, B², B³, and B⁴ is, independently, optionally substituted C₁-C₂ alkyl, optionally substituted C₁-C₃ heteroalkyl, optionally substituted C₂₋₉ heterocyclyl, O, S, S(O)₂, or NR^{N};
R^{N} is H, optionally substituted C₁₋₄ alkyl, optionally substituted C₂₋₄ alkenyl, optionally substituted C₂₋₄ alkynyl, optionally substituted C₂₋₉ heterocyclyl, optionally substituted C₆₋₁₂ aryl, or optionally substituted C₁₋₇ heteroalkyl;
each of C¹ and C² is, independently, carbonyl, thiocarbonyl, sulphonyl, or phosphoryl;
f, g, h, I, j, and k are each, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₉ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}-(C¹)_{g}-(B²)ₙ-to-(B³)ᵢ-(C²)ⱼ-(B⁴)ₖ-A².

In some embodiments, each of B¹, B², B³, and B⁴ is, independently, optionally substituted C₁-C₄ alkyl, optionally substituted C₁-C₄ heteroalkyl, or NR^{N}. In some embodiments, R^{N} is H or optionally substituted C₁₋₄ alkyl. In some embodiments, R^{N} is H or CH₃.

In some embodiments, each of B¹ and B⁴ is, independently,

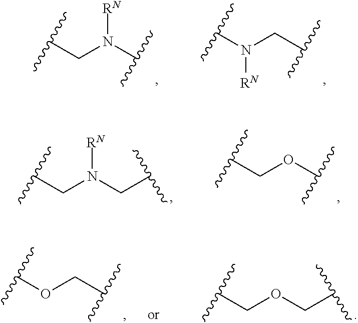

In some embodiments, B¹ is

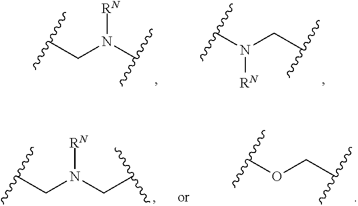

In some embodiments, each of C¹ and C² is, independently,

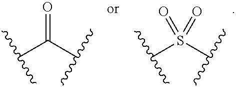

In some embodiments, C¹ is

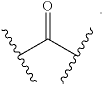 [SMILES: *C(*)=O]

In some embodiments, the chiral glutarimide has the structure:

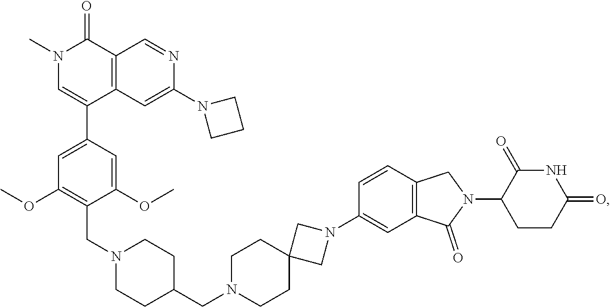 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N(C2CCC(=O)NC2=O)C4)C3)CC1]

In some embodiments, the chiral glutarimide has the structure:

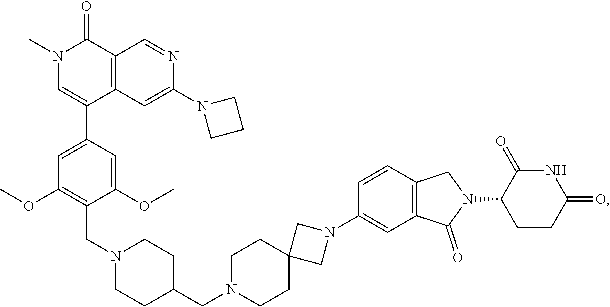 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N([C@H]2CCC(=O)NC2=O)C4)C3)CC1]

In some embodiments, the chiral glutarimide is enriched for one of the stereoisomers at the epimerizable stereogenic center.

In still further aspect, the invention provides a method of preparing a chiral glutarimide or a salt thereof including an epimerizable stereogenic center at the α-carbon of the glutarimide ring, the epimerizable stereogenic center being enriched for one of the stereoisomers, and the method including the step of reacting a stereoenriched aminoglutarimide with a carboxybenzaldehyde,
where the chiral glutarimide is of the following structure:

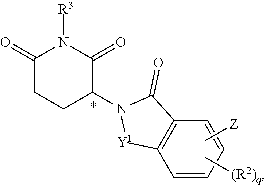

where
   *designates the stereoenriched epimerizable stereogenic center at the α-carbon of the glutarimide ring;
   Y¹ is

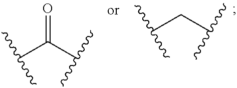

R³ is H, optionally substituted C₁-C₆ alkyl, or optionally substituted C₁-C₆ heteroalkyl;
q is 0, 1, 2, 3, or 4;
each R² is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; and
Z is L-A;
where L is a linker; and
A is a protein binding moiety;
where the enantioenriched aminoglutarimide is of the following structure:

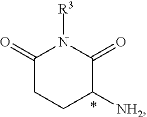

or a salt thereof,
where all variables are same as those in the chiral glutarimide;
   and
where the carboxybenzaldehyde is of the following structure:

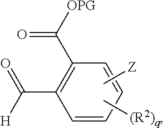

where
   PG is an O-protecting group, and
   all remaining variables are same as those in the chiral glutarimide.

In some embodiments, R³ is H or optionally substituted C₁-C₆ alkyl. In some embodiments, R³ is H or CH₃. In some embodiments, R³ is H. In some embodiments, R³ is CH₃. In some embodiments, Y¹ is

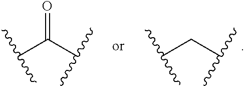

In some embodiments, Y¹ is

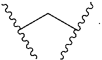 [SMILES: *C*]

In some embodiments, each R² is, independently, optionally substituted C₁-C₆ alkyl, optionally substituted C₁-C₆ heteroalkyl, hydroxyl, or optionally substituted amino. In some embodiments, q is 0 or 1. In some embodiments, q is 0. In some embodiments, the enantioenriched aminoglutarimide is reacted with the carboxybenzaldehyde under the reductive amination conditions.

In some embodiments, A has the structure of Formula E-3, Formula E-4, Formula G-2, Formula G-3, or Formula E-5:

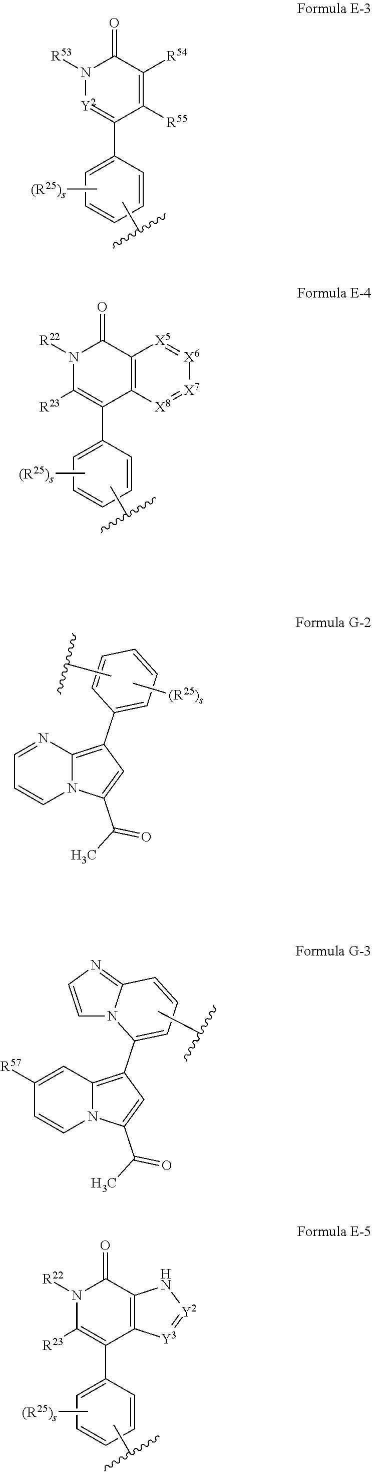

where
Y² is N or CR²³;
R²² is H, optionally substituted C₁-C₆ alkyl, or optionally substituted C₁-C₆ heteroalkyl;
R²³ is H, halogen, optionally substituted C₁-C₆ alkyl, or optionally substituted C₆-C₁₀ aryl;
s is 0, 1, 2, 3, or 4;
each R²⁵ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino;
R⁵³ is H, optionally substituted C₁-C₆ alkyl, optionally substituted C₁-C₆ heteroalkyl, or optionally substituted C₃-C₁₀ carbocyclyl;
R⁵⁴ is H or optionally substituted C₂-C₉ heteroaryl;
R⁵⁵ is H or N(R^{a})₂, where each R^{a} is independently H, optionally substituted C₁-C₆ alkyl, optionally substituted C₁-C₆ heteroalkyl, or optionally substituted C₃-C₁₀ carbocyclyl, or two geminal R^{a}, together with the nitrogen atom to which they are attached, combine to form optionally substituted C₂-C₉ heterocyclyl;
each of X⁵, X⁶, X⁷, and X⁸ is, independently, N or CR⁵⁶;
each R⁵⁶ is, independently, H or N(R^{a})₂, where Ra is H, optionally substituted C₁-C₆ alkyl, optionally substituted C₁-C₆ heteroalkyl, or optionally substituted C₃-C₁₀ carbocyclyl, or two geminal Ra, together with the nitrogen atom to which they are attached, combine to form optionally substituted C₂-C₉ heterocyclyl;
R⁵⁷ is optionally substituted C₂-C₁₀ heterocyclyl;
each of Y² and Y³ is, independently, N or CR⁵⁸; and
each R⁵⁸ is, independently, H or optionally substituted C₁-C₆ alkyl,
where if R⁵³ is H and R⁵⁴ is H, then R⁵⁵ is NR^{a}; if R⁵⁴ is H and R⁵⁵ is H, then R⁵³ is optionally substituted C₃-C₁₀ carbocyclyl; and if R⁵³ is H and R⁵⁵ is H, then R⁵⁴ is optionally substituted C₂-C₉ heteroaryl.

In some embodiments, A has the structure of Formula E-3. In some embodiments, A has the structure of Formula E-4. In some embodiments, A has the structure of Formula G-2. In some embodiments, A has the structure of Formula G-3. In some embodiments, A has the structure of Formula E-5.

In some embodiments, s is 0, 1, or 2.

In some embodiments, the linker has the structure of Formula II:

A¹-(B¹)_{f}-(C¹)_{g}-(B²)ₕ-(D)-(B³)ᵢ-(C²)ⱼ-(B⁴)ₖ-A² Formula II
where
A¹ is a bond between the linker and A;
A² is the valency of Z;
each of B¹, B², B³, and B⁴ is, independently, optionally substituted C₁-C₂ alkyl, optionally substituted C₁-C₃ heteroalkyl, optionally substituted C₂₋₉ heterocyclyl, O, S, S(O)₂, or NR^{N};
R^{N} is H, optionally substituted C₁₋₄ alkyl, optionally substituted C₂₋₄ alkenyl, optionally substituted C₂₋₄ alkynyl, optionally substituted C₂₋₉ heterocyclyl, optionally substituted C₆₋₁₂ aryl, or optionally substituted C₁₋₇ heteroalkyl;
each of C₁ and C₂ is, independently, carbonyl, thiocarbonyl, sulphonyl, or phosphoryl;
f, g, h, I, j, and k are each, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₉ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}-(C¹)_{g}-(B²)ₕ-to-(B³)ᵢ-(C²)ⱼ-(B⁴)ₖ-A².

In some embodiments, each of B¹, B², B³, and B⁴ is, independently, optionally substituted C₁-C₄ alkyl, optionally substituted C₁-C₄ heteroalkyl, or NR^{N}.

In some embodiments, R^{N} is H or optionally substituted C₁₋₄ alkyl. In some embodiments, R^{N} is H or CH₃.

In some embodiments, each of B¹ and B⁴ is, independently,

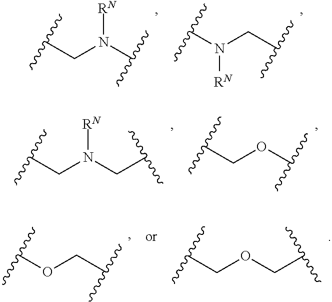

In some embodiments, B¹ is

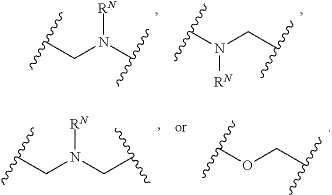

In some embodiments, each of C¹ and C² is, independently,

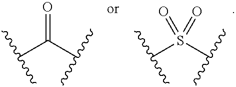

In some embodiments, C¹ is

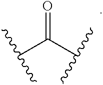 [SMILES: *C(*)=O]

In some embodiments, the carboxybenzaldehyde is of the following structure:

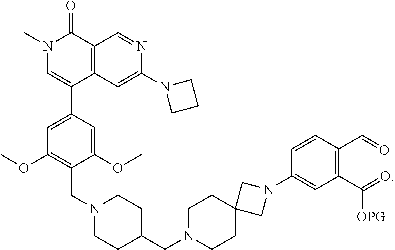

In some embodiments, the method further includes the step of preparing the carboxybenzaldehyde from a first reactant and a second reactant,

where the first reactant is of the following structure:

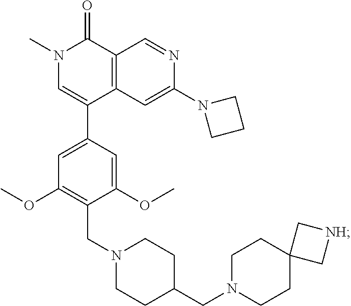 [SMILES: COc1cc(-c2cn(C)c(=O)c3cnc(N4CCC4)cc23)cc(OC)c1CN1CCC(CN2CCC3(CC2)CNC3)CC1]

and
where the second reactant is of the following structure:

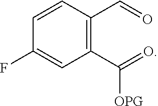

In some embodiments, the step of preparing the carboxybenzaldehyde is performed under the nucleophilic aromatic substitution reaction conditions.

In some embodiments, the method further includes the step of preparing the first reactant from a third reactant and a fourth reactant,

where the third reactant is a compound of the following structure:

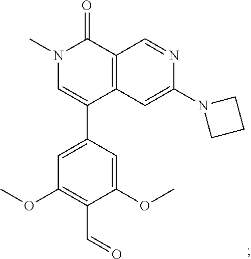 [SMILES: COc1cc(-c2cn(C)c(=O)c3cnc(N4CCC4)cc23)cc(OC)c1C=O]

and
where the fourth reactant is a compound of the following structure:

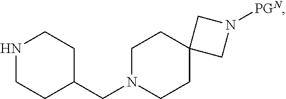

where PG^{N} is an N-protecting group.

In some embodiments, the step of preparing the first reactant includes reacting the third reactant and the fourth reactant under reductive amination reaction conditions and removing the N-protecting group.

In some embodiments, the method further includes the step of preparing the salt of the chiral glutarimide, where the step includes reacting a free-base form of the chiral glutarimide with an acid to produce the salt of the chiral glutarimide.

In some embodiments, the acid is citric acid, and the salt of the chiral glutarimide is a citrate salt of the chiral glutarimide.

### Chemical Terms

The term “acyl,” as used herein, represents a hydrogen or an alkyl group that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms).

An alkylene is a divalent alkyl group. The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the compounds described herein can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “bridged cyclyl,” as used herein, refers to a bridged polycyclic group of 5 to 20 atoms, containing from 1 to 3 bridges. Bridged cyclyl includes bridged carbocyclyl (e.g., norbornyl) and bridged heterocyclyl (e.g., 1,4-diazabicyclo[2.2.2]octane).

The term “cyano,” as used herein, represents a-CN group.

The term “carbocyclyl,” as used herein, refers to a non-aromatic C₃-C₁₂ monocyclic or polycyclic (e.g., bicyclic or tricyclic) structure in which the rings are formed by carbon atoms. Carbocyclyl structures include cycloalkyl groups and unsaturated carbocyclyl radicals. Polycyclic carbocyclyl includes spirocyclic carbocyclyl, bridged carbocyclyl, and fused carbocyclyl.

The term “cycloalkyl,” as used herein, refers to a saturated, non-aromatic, monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

The term “halogen,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic monocyclic or polycyclic structure of 5 to 12 atoms having at least one aromatic ring containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, refers a monocyclic or polycyclic (e.g., bicyclic or tricyclic) structure having 3 to 12 atoms having at least one ring containing 1, 2, 3, or 4 ring atoms selected from N, O or S and no aromatic ring containing any N, O, or S atoms. Polycyclic heterocyclyl includes spirocyclic heterocyclyl, bridged heterocyclyl, and fused heterocyclyl. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyalkyl,” as used herein, represents alkyl group substituted with an-OH group.

The term “hydroxyl,” as used herein, represents an-OH group.

The term “nitro,” as used herein, represents an-NO₂ group.

The term “protecting group,” as used herein, represents a group intended to protect a hydroxy, an amino, or a carbonyl from participating in one or more undesirable reactions during chemical synthesis. The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino, amido, heterocyclic N—H, or hydrazine) group from participating in one or more undesirable reactions during chemical synthesis. Commonly used O- and N-protecting groups are disclosed in Greene, “Protective Groups in Organic Synthesis,” 3rd Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, a-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p methoxybenzyloxycarbonyl, p-nitrobenzyloxycarbonyl, 2-nitrobenzyloxycarbonyl, p bromobenzyloxycarbonyl, 3,4-dimethoxybenzyloxycarbonyl, 3,5 dimethoxybenzyl oxycarbonyl, 2,4-dimethoxybenzyloxycarbonyl, 4 methoxybenzyloxycarbonyl, 2-nitro-4,5-dimethoxybenzyloxycarbonyl, 3,4,5 trimethoxybenzyloxycarbonyl, 1-(p-biphenylyl)-1-methylethoxycarbonyl, α,α-dimethyl-3,5 dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, p-methoxybenzyl, 2,4-dimethoxybenzyl, triphenylmethyl, benzyloxymethyl, and the like, silylalkylacetal groups such as [2-(trimethylsilyl)ethoxy]methyl and silyl groups such as trimethylsilyl, and the like. Useful N-protecting groups are formyl, acetyl, benzoyl, pivaloyl, t-butylacetyl, alanyl, phenylsulfonyl, benzyl, dimethoxybenzyl, [2-(trimethylsilyl)ethoxy]methyl (SEM), tetrahydropyranyl (THP), t-butyloxycarbonyl (Boc), and benzyloxycarbonyl (Cbz).

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxyl), aryl (e.g., substituted and unsubstituted phenyl), carbocyclyl (e.g., substituted and unsubstituted cycloalkyl), halogen (e.g., fluoro), hydroxyl, oxo (═O), heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds described herein can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, where such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R”,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds described herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “including” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “degradation moiety” refers to a moiety whose binding results in degradation of a protein, e.g., BRD9. In one example, the moiety binds to a protease or a ubiquitin ligase that metabolizes the protein, e.g., BRD9.

As used herein, the term “epimerizable” refers to a property of a stereogenic center to undergo inversion in certain polar liquids at room temperature.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid. The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

As used herein, the term “stereochemically enriched” refers to a composition containing a plurality of compounds having the same connectivity and differing from each other by their stereochemical identities, such that those compounds having a particular stereochemical feature (e.g., stereogenic center) in excess of its opposite are considered to be stereochemically enriched. The stereochemical enrichment may be, e.g., by at least 20% in favor of the stereochemical feature that is stereochemically enriched (e.g., diastereomeric or enantiomeric excess of at least 10%, at least 50%, at least 70%, at least 80%, at least 90%, at least 95%, at least 98%, or at least 99%; up to diastereopure or enantiopure). A stereochemically enriched compound that has only one stereogenic center may also be called as “enantioenriched.”

As used herein, the term “subject” refers to a mammal (e.g., a human, non-human primate, dog, pig, guinea pig, rat, or mouse). Typically, subjects in the present disclosure are those that have been previously administered a chiral glutarimide, e.g., as part of ADME or PK studies.

### BRIEF DESCRIPTION OF THE DRAWINGS

FIG. 1 is an image illustrating dose dependent depletion of BRD9 levels in a synovial sarcoma cell line (SYO1) in the presence of a BRD9 degrader.

FIG. 2 is an image illustrating sustained suppression of BRD9 levels in a synovial sarcoma cell line (SYO1) in the presence of a BRD9 degrader over 72 hours.

FIG. 3 is an image illustrating sustained suppression of BRD9 levels in two cell lines (293T and SYO1) in the presence of a BRD9 degrader over 5 days.

FIG. 4 is an image illustrating sustained suppression of BRD9 levels in synovial sarcoma cell lines (SYO1 and Yamato) in the presence of a BRD9 degrader over 7 days compared to the levels in cells treated with CRISPR reagents.

FIG. 5 is an image illustrating the effect on cell growth of six cell lines (SYO1, Yamato, A549, HS-SY-II, ASKA, and 293T) in the presence of a BRD9 degrader and a BRD9 inhibitor.

FIG. 6 is an image illustrating the effect on cell growth of two cell lines (SYO1 and G401) in the presence of a BRD9 degrader.

FIG. 7 is an image illustrating the effect on cell growth of three synovial sarcoma cell lines (SYO1, HS-SY-II, and ASKA) in the presence of a BRD9 degrader, BRD9 binder and E3 ligase binder.

FIG. 8 is an image illustrating the effect on cell growth of three non-synovial sarcoma cell lines (RD, HCT116, and Calu6) in the presence of a BRD9 degrader, BRD9 binder and E3 ligase binder.

FIG. 9 is a graph illustrating the percentage of SYO1 in various cell cycle phases following treatment with DMSO, Compound 1 at 200 nM, or Compound 1 at 1 μM for 8 or 13 days.

FIG. 10 is a series of contour plots illustrating the percentage of SYO1 cells in various cell cycle phases following treatment with DMSO, Compound 1 at 200 nM, Compound 1 at 1 μM, or lenalidomide at 200 nM for 8 days. Numerical values corresponding to each contour plot are found in the table below.

FIG. 11 is a series of contour plots illustrating the percentage of SYO1 cells in various cell cycle phases following treatment with DMSO, Compound 1 at 200 nM, Compound 1 at 1 UM, or lenalidomide at 200 nM for 13 days. Numerical values corresponding to each contour plot are found in the table below.

FIG. 12 is a series of contour plots illustrating the percentage of early- and late-apoptotic SYO1 cells following treatment with DMSO, Compound 1 at 200 nM, Compound 1 at 1 μM, or lenalidomide at 200 nM for 8 days. Numerical values corresponding to each contour plot are found in the table below.

FIG. 13 is a graph illustrating the proteins present in BAF complexes including the SS18-SSX fusion protein.

FIG. 14 is a graph showing efficacy of compound D1 in SOY-1 xenograft mouse model. Treatment with compound D1 led to tumor growth inhibition.

FIG. 15 is an image of a western blot showing BRD9 detection in the control group and the treatment group (compound D1). Treatment with compound D1 led to BRD9 inhibition.

FIG. 16 is an image of western blots showing BRD9 detection in the SYO-1 cells treated with DMSO, Enantiomer 1, or racemic compound D1 for 1 or 6 hours.

FIG. 17 is an image of western blots showing BRD9 detection in the SYO-1 cells treated with DMSO, Enantiomer 2, or racemic compound D1 for 1 or 6 hours.

FIG. 18 is a graph showing dose response curves fitted to BRD9 band intensity data points from western blot images illustrated in FIGS. 16 and 17.

FIG. 19 is an image of western blots showing BRD9 detection in the SYO-1 cells treated with Enantiomer 1, Enantiomer 2, or racemic compound D1 for 24 hours.

FIG. 20 is an image of western blots showing BRD9 detection in the ASKA cell controls and the ASKA cells treated with Enantiomer 1 or racemic compound D1 for 0.5 or 2 hours.

FIG. 21 is an image of western blots showing BRD9 detection in the ASKA cell controls and the ASKA cells treated with Enantiomer 2 or racemic compound D1 for 0.5 or 2 hours.

FIG. 22 is a graph showing dose response curves fitted to BRD9 band intensity data points from western blot images illustrated in FIGS. 20 and 21.

FIG. 23 are images showing a series of western blots for BRD9 detection in SYO-1 Zenograft model treated with Enantiomer 1, Enantiomer 2, or racemic compound D1.

FIG. 24 is a bar graph quantifying the BRD9 level changes observed in western blots illustrated in FIG. 23.

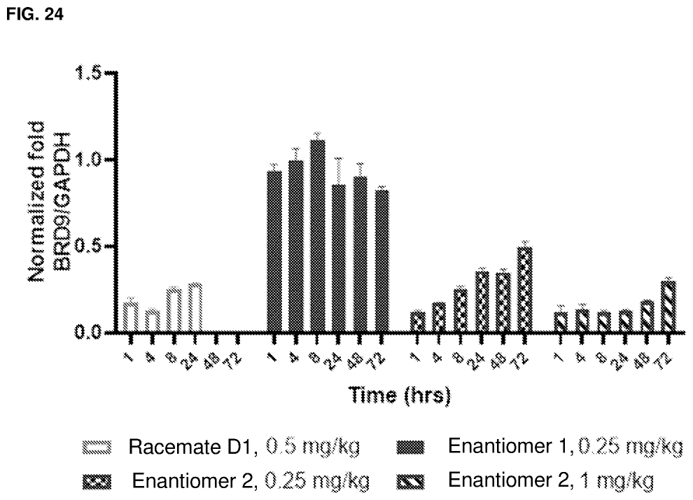

### DETAILED DESCRIPTION

The invention provides methods and compositions useful for preparing or storing a chiral glutarimide having an epimerizable stereogenic center at the α-carbon of the glutarimide ring, while reducing or eliminating epimerization of the epimerizable stereogenic center. Thus, methods and compositions disclosed herein may reduce or eliminate erosion of the stereochemical enrichment of the chiral glutarimide at the epimerizable stereogenic center at the α-carbon of the glutarimide ring.

The methods and compositions described herein take advantage of the stabilizing effect of citric acid upon the stereochemistry of the epimerizable stereogenic center at the α-carbon of the glutarimide ring in the chiral glutarimide. Thus, the methods and compositions may be used to slow down significantly epimerization at the α-carbon of the glutarimide ring in chiral glutarimides, e.g., in certain media (e.g., blood or a component thereof, such as blood plasma or blood serum) otherwise capable of promoting such epimerization and concomitant erosion of the stereochemical enrichment. The methods described herein include methods of determining the level of a chiral glutarimide including an epimerizable stereogenic center at the α-carbon of the glutarimide ring in a subject; methods of preparing a composition including a chiral glutarimide including an epimerizable stereogenic center at the α-carbon of the glutarimide ring and a body fluid; and methods of preparing a composition including a chiral glutarimide including an epimerizable stereogenic center at the α-carbon of the glutarimide ring and a component of a body fluid.

The invention also provides a stereoretentive synthesis of a chiral glutarimide. Advantageously, the synthesis approach described herein introduces the glutarimide ring at the end of the synthesis and reduces exposure of the epimerizable stereogenic center at the α-carbon of the glutarimide ring to various reaction, work up, and purification conditions, which could promote epimerization.

### Stability-Enhancing Compositions and Methods

The compositions and methods disclosed herein typically combine a chiral glutarimide, a body fluid (e.g., blood) or a component thereof (e.g., blood plasma or blood serum), and citric acid. A chiral glutarimide includes an epimerizable stereogenic center at the α-carbon of the glutarimide ring that is typically stereochemically enriched for one of the stereoisomeric forms of the stereogenic center (e.g., S or R). For example, in compositions and methods disclosed herein, the epimerizable stereogenic center at the α-carbon of the glutarimide may be stereochemically enriched by at least 20% in favor of the stereochemical orientation (e.g., S) that is stereochemically enriched (e.g., stereochemical excess of at least 10%, at least 50%, at least 70%, at least 80%, at least 90%, at least 95%, at least 98%, or at least 99%; up to stereochemically pure). Thus, compositions and method disclosed herein may be substantially stereochemical enrichment-preserving for an epimerizable stereogenic center at the a-carbon of the glutarimide ring.

Preparation of a composition containing a body fluid (e.g., blood) and the chiral glutarimide (e.g., as a subject's blood sample, e.g., the subject having been administered the chiral glutarimide) typically includes the step of collecting the body fluid from a subject into a container including a solution (e.g., an aqueous solution) containing citric acid (e.g., 0.5 M to 5 M citric acid or 3 M citric acid).

Preparation of a composition containing a body fluid component (e.g., blood plasma or blood serum) and the chiral glutarimide (e.g., as a subject's blood plasma or serum sample, e.g., the subject having been administered the chiral glutarimide) typically includes the step of combining citric acid and the component of the body fluid collected from a subject. For the combining step, citric acid may be, e.g., in lyophilized form. The methods described herein for the preparation of a composition containing a body fluid component may also include the step of preparing the body fluid component from the body fluid collected from the subject. The component of the body fluid may be prepared from the body fluid using methods known in the art. For example, blood plasma may be prepared by centrifugation of blood; blood serum may be prepared by clotting and subsequent centrifugation of blood. The methods described herein for the preparation of a composition containing a body fluid component may further include the step of collecting the body fluid from the subject (e.g., the subject having been administered the chiral glutarimide). The body fluid collections methods are known in the art and, e.g., for blood, typically involve arterial sampling, venipuncture sampling, or fingerstick sampling. The body fluid may be stored at 0° C. to 4° C. (e.g., in wet-ice bath) between the step of collecting the body fluid and the step of preparing the component of the body fluid (e.g., for up to 2 hours). Typically, the body fluid component is combined with citric acid immediately after the component's preparation.

In the compositions and methods described herein, the concentration of citric acid may be, e.g., 0.005M to 0.05M (e.g., 0.03M) after the citric acid is combined with blood or a component thereof.

### Chiral Glutarimides

The compositions and methods described herein typically contain a chiral glutarimide having an epimerizable stereogenic center at the α-carbon of the glutarimide ring. Such chiral glutarimides are typically of the following structure:

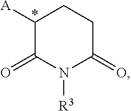

where
*designates the stereoenriched epimerizable stereogenic center at the α-carbon of the glutarimide ring;
A is a non-hydrogen group; and
R³ is H, optionally substituted C₁-C₆ alkyl, or optionally substituted C₁-C₆ heteroalkyl.
The chiral glutarimides can often be used as a Cereblon ligand, e.g., to be used as degraders for targeted protein degradation. Accordingly, the chiral glutarimide may be, e.g., a compound of formula I:

A-L-B Formula I,
where
L is a linker;
B is a degradation moiety including a Cereblon ligand including an epimerizable stereogenic center at the α-carbon of the glutarimide ring in the Cereblon ligand; and

A has the structure of Formula E-3, Formula E-4, Formula G-2, Formula G-3, or Formula E-5:

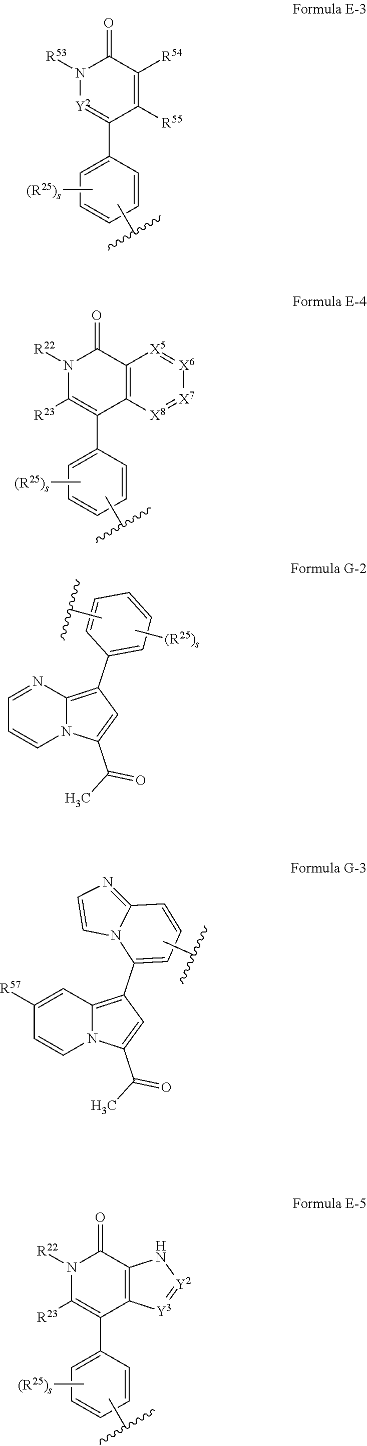

where
Y² is N or CR²³;
R²² is H, optionally substituted C₁-C₆ alkyl, or optionally substituted C₁-C₆ heteroalkyl;
R²³ is H, halogen, optionally substituted C₁-C₆ alkyl, or optionally substituted C₆-C₁₀ aryl;
s is 0, 1, 2, 3, or 4;
each R²⁵ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino;
R⁵³ is H, optionally substituted C₁-C₆ alkyl, optionally substituted C₁-C₆ heteroalkyl, or optionally substituted C₃-C₁₀ carbocyclyl;
R⁵⁴ is H or optionally substituted C₂-C₉ heteroaryl;
R⁵⁵ is H or N(R^{a})₂, wherein each R^{a} is independently H, optionally substituted C₁-C₆ alkyl, optionally substituted C₁-C₆ heteroalkyl, or optionally substituted C₃-C₁₀ carbocyclyl, or two geminal Ra, together with the nitrogen atom to which they are attached, combine to form optionally substituted C₂-C₉ heterocyclyl;
each of X⁵, X⁶, X⁷, and X⁸ is, independently, N or CR⁵⁶;
each R⁵⁶ is, independently, H or N(R^{a})₂, wherein R^{a} is H, optionally substituted C₁-C₆ alkyl, optionally substituted C₁-C₆ heteroalkyl, or optionally substituted C₃-C₁₀ carbocyclyl, or two geminal R^{a}, together with the nitrogen atom to which they are attached, combine to form optionally substituted C₂-C₉ heterocyclyl;
R⁵⁷ is optionally substituted C₂-C₁₀ heterocyclyl;
each of Y² and Y³ is, independently, N or CR⁵⁸; and
each R⁵⁸ is, independently, H or optionally substituted C₁-C₆ alkyl,
wherein if R⁵³ is H and R⁵⁴ is H, then R⁵⁵ is NR^{a}; if R⁵⁴ is H and R⁵⁵ is H, then R⁵³ is optionally substituted C₃-C₁₀ carbocyclyl; and if R⁵³ is H and R⁵⁵ is H, then R⁵⁴ is optionally substituted C₂-C₉ heteroaryl,
or a pharmaceutically acceptable salt thereof.

In some embodiments, A has the structure of Formula E-3. In some embodiments, A has the structure of Formula E-4. In some embodiments, A has the structure of Formula G-2. In some embodiments, A has the structure of Formula G-3. In some embodiments, A has the structure of Formula E-5. In some embodiments, s is 0, 1, or 2.

In some embodiments, the degradation moiety has the structure of Formula A-1:

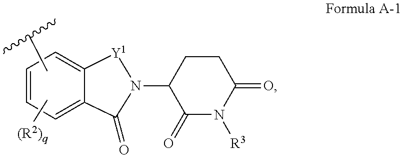

wherein

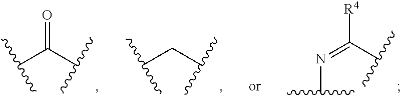

Y¹ is
R³ and R⁴ are, independently, H, optionally substituted C₁-C₆ alkyl, or optionally substituted C₁-C₆ heteroalkyl;
q is 0, 1, 2, 3, or 4; and
each R₂ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino,
or a pharmaceutically acceptable salt thereof.

In some embodiments, the degradation moiety has the structure of Formula A-1a:

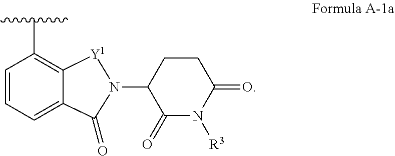

In some embodiments, the degradation moiety has the structure of Formula A-1b:

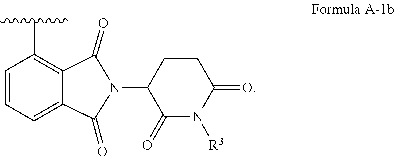

In some embodiments, the degradation moiety has the structure of Formula A-1c:

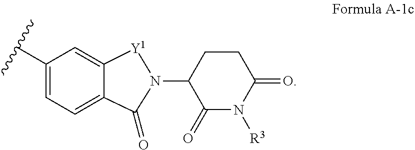

In some embodiments, the degradation moiety has the structure of Formula A-1d:

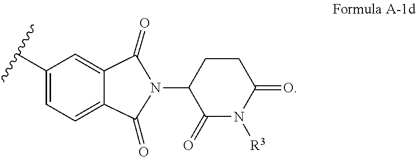

In some embodiments, the degradation moiety has the structure:

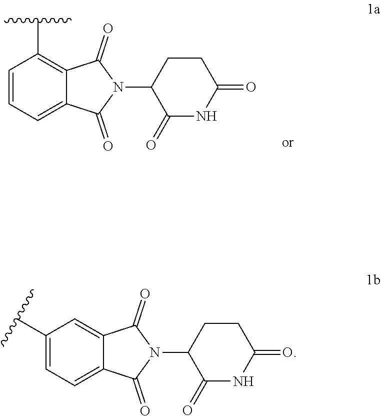 [SMILES: *c1cccc2c1C(=O)N(C1CCC(=O)NC1=O)C2=O ; *c1ccc2c(c1)C(=O)N(C1CCC(=O)NC1=O)C2=O]

In some embodiments, the linker has the structure of Formula II:

A¹-(B¹)_{f}-(C¹)_{g}-(B²)ₕ-(D)-(B³)ᵢ-(C₂)ⱼ-(B⁴)ₖ-A² Formula II
wherein
A¹ is a bond between the linker and A;
A² is a bond between B and the linker;
each of B¹, B², B³, and B⁴ is, independently, optionally substituted C₁-C₂ alkyl, optionally substituted C₁-C₃ heteroalkyl, optionally substituted C₂₋₉ heterocyclyl, O, S, S(O)₂, or NR^{N};
R^{N} is H, optionally substituted C₁₋₄ alkyl, optionally substituted C₂₋₄ alkenyl, optionally substituted C₂₋₄ alkynyl, optionally substituted C₂₋₉ heterocyclyl, optionally substituted C₆₋₁₂ aryl, or optionally substituted C₁₋₇ heteroalkyl;
each of C¹ and C² is, independently, carbonyl, thiocarbonyl, sulphonyl, or phosphoryl;
f, g, h, I, j, and k are each, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₉ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}-(C¹)_{g}-(B²)ₕ-to-(B³)ᵢ-(C²)ⱼ-(B⁴)ₖ-A².

In some embodiments, the chiral glutarimide is compound S-D1:

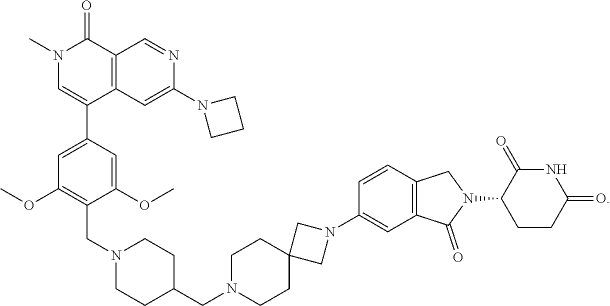 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N([C@H]2CCC(=O)NC2=O)C4)C3)CC1]

### Stereoretentive Synthesis of Chiral Glutarimides

The invention further provides stereoretentive approaches for the synthesis of chiral glutarimides. The methods disclosed herein thus may be used to prepare a chiral glutarimide comprising an epimerizable stereogenic center at the α-carbon of the glutarimide ring, the epimerizable stereogenic center being enriched for one of the stereoisomers. The method typically include the step of reacting an stereoenriched aminoglutarimide with a carboxybenzaldehyde, wherein the chiral glutarimide is of the following structure:

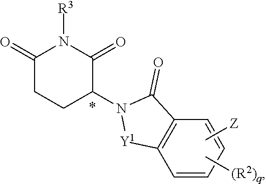

wherein
   *designates the stereoenriched epimerizable stereogenic center at the α-carbon of the glutarimide ring;
   Y¹ is

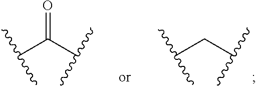

R³ is H, optionally substituted C₁-C₆ alkyl, or optionally substituted C₁-C₆ heteroalkyl;
q is 0, 1, 2, 3, or 4;
each R₂ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; and
Z is a substituent;
   wherein the enantioenriched aminoglutarimide is of the following structure:

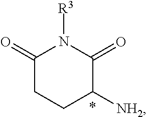

or a salt thereof,
wherein all variables are same as those in the chiral glutarimide;
   and
   wherein the carboxybenzaldehyde is of the following structure:

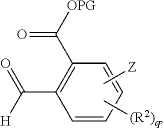

wherein
   PG is an O-protecting group (e.g., alkyl, such as methyl), and
   all remaining variables are same as those in the chiral glutarimide.

In some embodiments, the enantioenriched aminoglutarimide is reacted with the carboxybenzaldehyde under the reductive amination conditions. Reductive amination reaction conditions are known in the art. Typically, reductive amination involves a reaction between a carbonyl functional group in an aldehyde or ketone with a primary or secondary amine in the presence of a 1,2-reducing agent (e.g., NaBH₃CN, NaBH(OAc)₃, or NaBH₄/acetic acid) to produce a secondary or tertiary amine, respectively.

In some embodiments, Z is -L-A,

where
L is a linker, and
A has the structure of Formula E-3, Formula E-4, Formula G-2, Formula G-3, or Formula E-5:

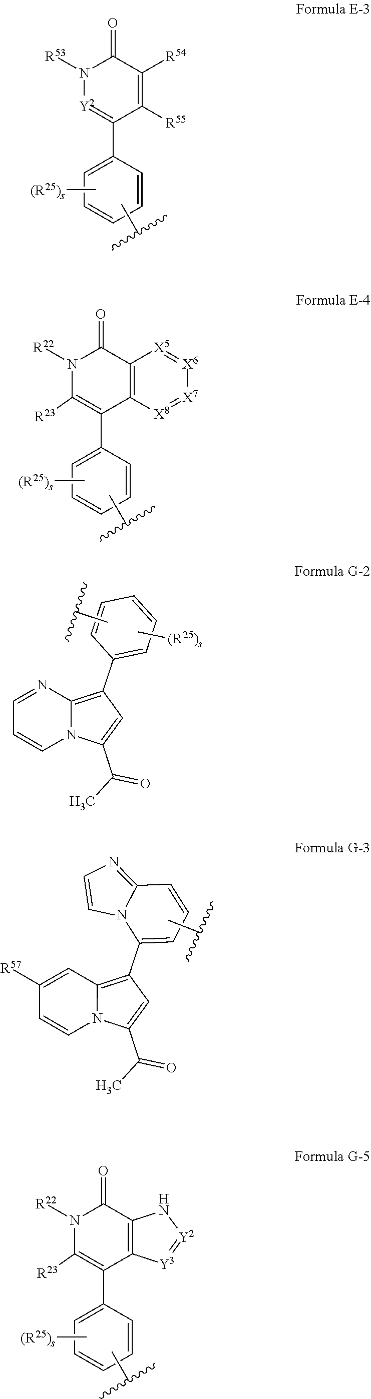

wherein
Y² is N or CR²³;
R²² is H, optionally substituted C₁-C₆ alkyl, or optionally substituted C₁-C₆ heteroalkyl;
R²³ is H, halogen, optionally substituted C₁-C₆ alkyl, or optionally substituted C₆-C₁₀ aryl;
s is 0, 1, 2, 3, or 4;
each R²⁵ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino;
R⁵³ is H, optionally substituted C₁-C₆ alkyl, optionally substituted C₁-C₆ heteroalkyl, or optionally substituted C₃-C₁₀ carbocyclyl;
R⁵⁴ is H or optionally substituted C₂-C₉ heteroaryl;
R⁵⁵ is H or N(R^{a})₂, wherein each R^{a} is independently H, optionally substituted C₁-C₆ alkyl, optionally substituted C₁-C₆ heteroalkyl, or optionally substituted C₃-C₁₀ carbocyclyl, or two geminal R^{a}, together with the nitrogen atom to which they are attached, combine to form optionally substituted C₂-C₉ heterocyclyl;
each of X⁵, X⁸, X⁷, and X⁸ is, independently, N or CR⁵⁶;
each R⁵⁶ is, independently, H or N(R^{a})₂, wherein R^{a} is H, optionally substituted C₁-C₆ alkyl, optionally substituted C₁-C₆ heteroalkyl, or optionally substituted C₃-C₁₀ carbocyclyl, or two geminal R^{a}, together with the nitrogen atom to which they are attached, combine to form optionally substituted C₂-C₉ heterocyclyl;
R⁵⁷ is optionally substituted C₂-C₁₀ heterocyclyl;
each of Y² and Y³ is, independently, N or CR⁵⁸; and
each R⁵⁸ is, independently, H or optionally substituted C₁-C₆ alkyl,
wherein if R⁵³ is H and R⁵⁴ is H, then R⁵⁵ is NR^{a}; if R⁵⁴ is H and R⁵⁵ is H, then R⁵³ is optionally substituted C₃-C₁₀ carbocyclyl; and if R⁵³ is H and R⁵⁵ is H, then R⁵⁴ is optionally substituted C₂-C₉ heteroaryl.

In some embodiments, the linker has the structure of Formula II:

A¹-(B¹)_{f}-(C¹)_{g}-(B²)ₕ-(D)-(B³)ᵢ-(C²)ⱼ-(B⁴)ₖ-A² Formula II
wherein
A¹ is a bond between the linker and A;
A² is the valency of Z;
each of B¹, B², B³, and B⁴ is, independently, optionally substituted C₁-C₂ alkyl, optionally substituted C₁-C₃ heteroalkyl, optionally substituted C₂₋₉ heterocyclyl, O, S, S(O)₂, or NR^{N};
R^{N} is H, optionally substituted C₁₋₄ alkyl, optionally substituted C₂₋₄ alkenyl, optionally substituted C₂₋₄ alkynyl, optionally substituted C₂₋₉ heterocyclyl, optionally substituted C₆₋₁₂ aryl, or optionally substituted C₁₋₇ heteroalkyl;
each of C¹ and C² is, independently, carbonyl, thiocarbonyl, sulphonyl, or phosphoryl;
f, g, h, I, j, and k are each, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₉ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}-(C¹)_{g}-(B²)ₙ-to-(B³)ᵢ-(C²)ⱼ-(B⁴)ₖ-A².

In some embodiments, the carboxybenzaldehyde is of the following structure:

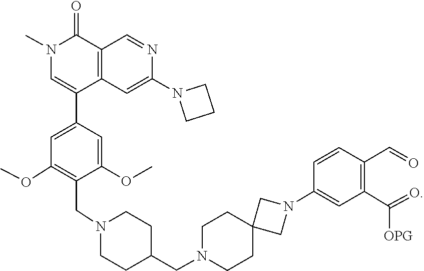

In some embodiments, the method further comprises the step of preparing the carboxybenzaldehyde from a first reactant and a second reactant, wherein the first reactant is of the following structure:

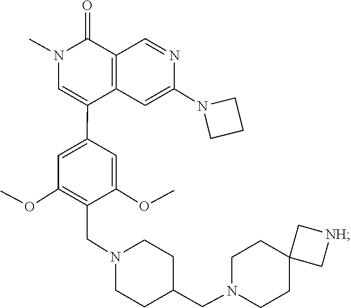 [SMILES: COc1cc(-c2cn(C)c(=O)c3cnc(N4CCC4)cc23)cc(OC)c1CN1CCC(CN2CCC3(CC2)CNC3)CC1]

and
wherein the second reactant is of the following structure:

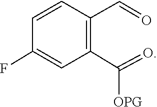

In some embodiments, the step of preparing the carboxybenzaldehyde is performed under the nucleophilic aromatic substitution reaction conditions.

In some embodiments, the method further comprises the step of preparing the first reactant from a third reactant and a fourth reactant,

wherein the third reactant is a compound of the following structure:

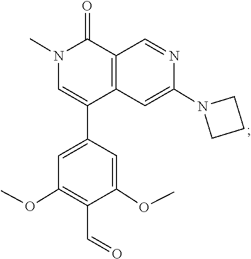 [SMILES: COc1cc(-c2cn(C)c(=O)c3cnc(N4CCC4)cc23)cc(OC)c1C=O]

and
wherein the fourth reactant is a compound of the following structure:

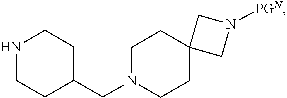

wherein PG^{N} is an N-protecting group (e.g., Boc).

In some embodiments, the step of preparing the first reactant comprises reacting the third reactant and the fourth reactant under reductive amination reaction conditions and removing the N-protecting group. N-protecting groups can be removed methods known in the art.

In some embodiments, the method further comprises the step of preparing the salt of the chiral glutarimide, wherein the step comprises reacting a free-base form of the chiral glutarimide with an acid to produce the salt of the chiral glutarimide. In some embodiments, the acid is citric acid, and the salt of the chiral glutarimide is a citrate salt of the chiral glutarimide.

### EXAMPLES

Throughout the Examples, compound numbers are as shown in Table 1.

TABLE 1
Compounds

| Compound | |
|---|---|
| No. | Structure |
| D1 | 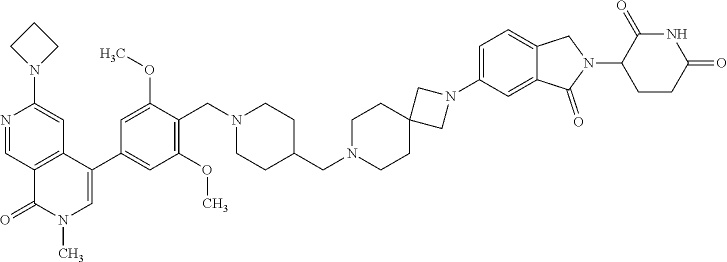 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N(C2CCC(=O)NC2=O)C4)C3)CC1] |
| S-D1 | 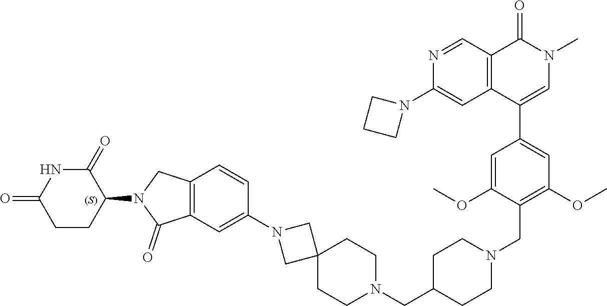 |
| R-D1 | 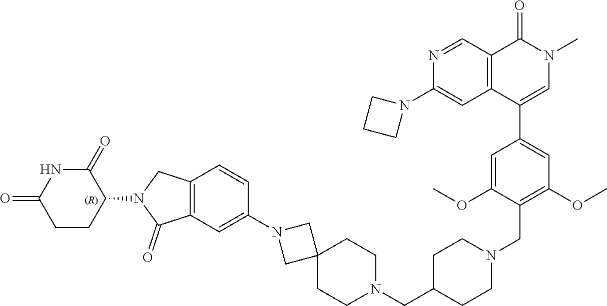 |

Examples 6-8 illustrate the preparation of compounds D1, as well as compounds S-D1 and R-D1 through resolution of racemate D1. Example

### Example 1-BRD9 Degrader Depletes BRD9 Protein

The following example demonstrates the depletion of the BRD9 protein in synovial sarcoma cells treated with a BRD9 degrader.

Procedure: Cells were treated with DMSO or the BRD9 degrader, Compound 1 (also known as dBRD9, see Remillard et al, Angew. Chem. Int. Ed. Engl. 56 (21): 5738-5743 (2017); see structure of compound 1 below), for indicated doses and timepoints.

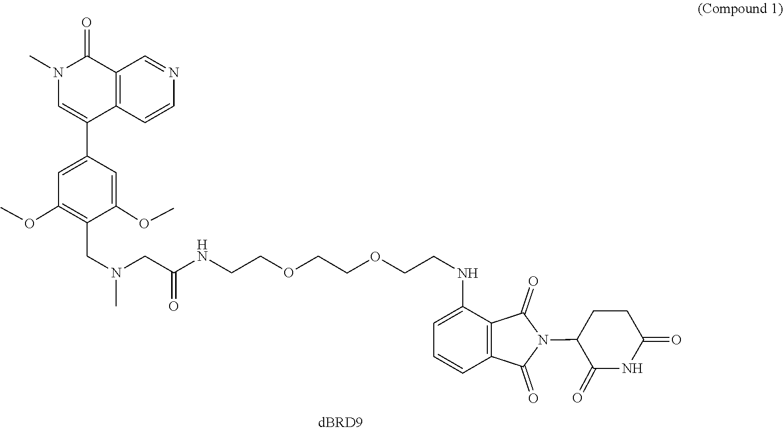 [SMILES: COc1cc(-c2cn(C)c(=O)c3cnccc23)cc(OC)c1CN(C)CC(=O)NCCOCCOCCNc1cccc2c1C(=O)N(C1CCC(=O)NC1=O)C2=O]

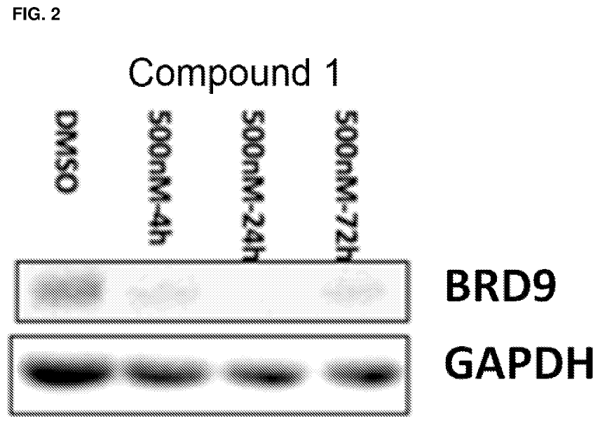

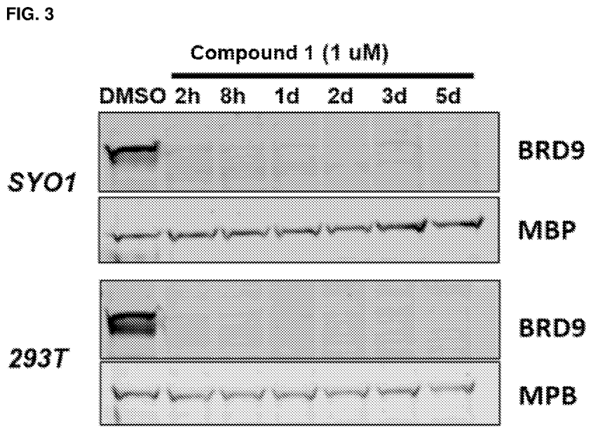

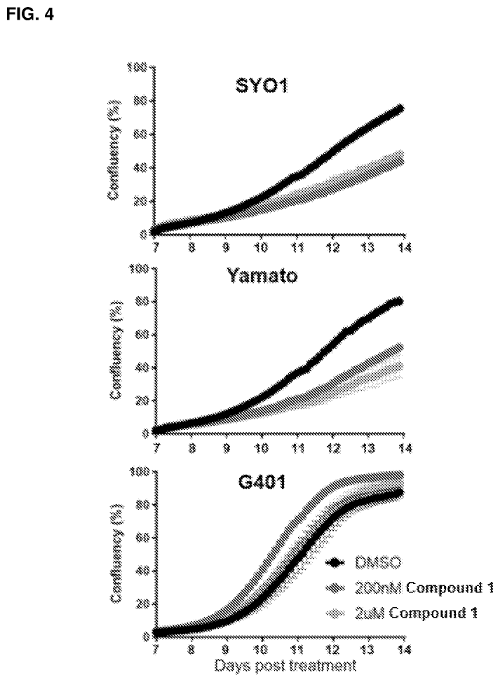

Whole cell extracts were fractionated by SDS-PAGE and transferred to a polyvinylidene difluoride membrane using a transfer apparatus according to the manufacturer's protocols (Bio-Rad). After incubation with 5% nonfat milk in TBST (10 mM Tris, pH 8.0, 150 mM NaCl, 0.5% Tween 20) for 60 min, the membrane was incubated with antibodies against BRD9 (1:1,000, Bethyl laboratory A303-781A), GAPDH (1:5,000, Cell Signaling Technology), and/or MBP (1:1,000, BioRad) overnight at 4° C. Membranes were washed three times for 10 min and incubated with anti-mouse or anti-rabbit antibodies conjugated with either horseradish peroxidase (HRP, FIGS. 2-3) or IRDye (FIG. 4, 1:20,000, LI-COR) for at least 1 h. Blots were washed with TBST three times and developed with either the ECL system according to the manufacturer's protocols (FIGS. 2-3) or scanned on an Odyssey CLx Imaging system (FIG. 4).

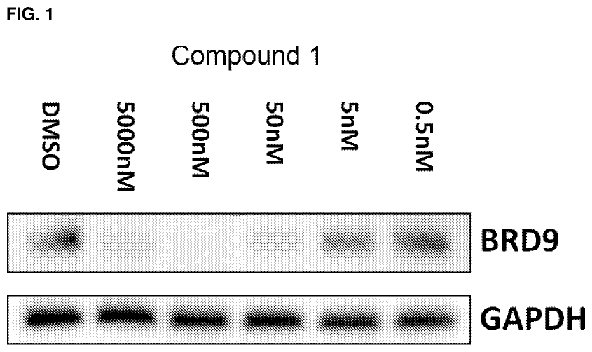

Results: Treatment of SYO1 synovial sarcoma cells with the BRD9 degrader Compound 1 results in dose dependent (FIG. 1) and time dependent (FIG. 2) depletion of BRD9 in the cells. Further, as shown in FIG. 3, the depletion of BRD9 by Compound 1 is replicated in a non-synovial sarcoma cell line (293T) and may be sustained for at least 5 days.

### Example 2—Inhibition of Growth of Synovial Cell Lines by BRD9 Inhibitors and BRD9 Degraders

The following example demonstrates that BRD9 degraders and inhibitors selectively inhibit growth of synovial sarcoma cells.

### Procedures:

Cells were treated with DMSO or the BRD9 degrader, Compound 1, at indicated concentrations, and proliferation was monitored from day 7 to day 14 by measuring confluency over time using an IncuCyte live cell analysis system (FIG. 4). Growth medium and compounds were refreshed every 3-4 days.

Cells were seeded into 12-well plates and treated with DMSO, 1 μM BRD9 inhibitor, Compound 2 (also known as BI-7273, see Martin et al, J Med Chem. 59 (10): 4462-4475 (2016); see structure of compound 2 below), or 1 μM BRD9 degrader, Compound 1.

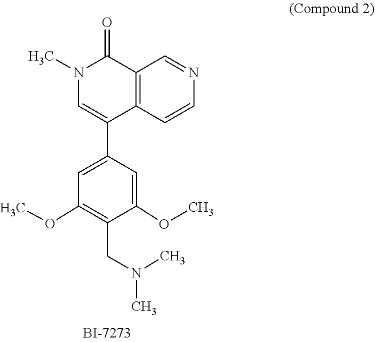 [SMILES: COc1cc(-c2cn(C)c(=O)c3cnccc23)cc(OC)c1CN(C)C]

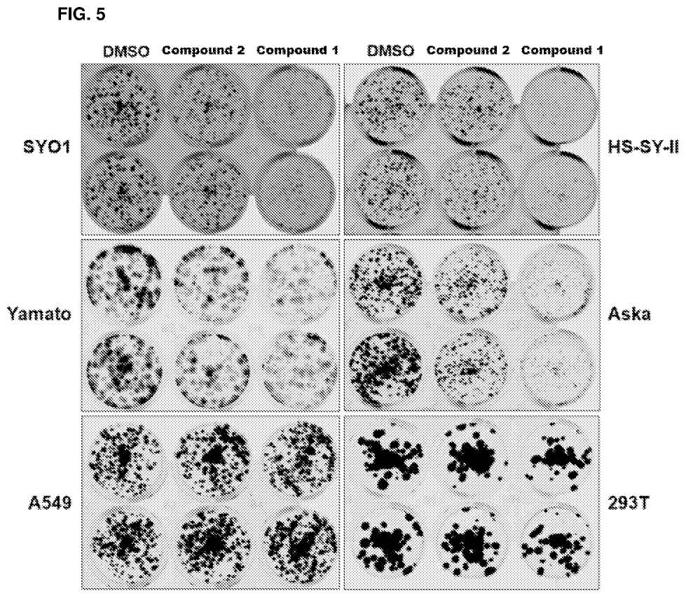

The number of cells was optimized for each cell line. Growth medium and compounds were refreshed every 3-5 days. SYO1, Yamato, A549, 293T and HS-SY-II cells were fixed and stained at day 11. ASKA cells were fixed and stained at day 23. Staining was done by incubation with crystal violet solution (0.5 g Crystal Violet, 27 ml 37% Formaldehyde, 100 mL 10×PBS, 10 mL Methanol, 863 dH20 to 1 L) for 30 min followed by 3×washes with water and drying the plates for at least 24 h at room temperature. Subsequently plates were scanned on an Odyssey CLx Imaging system (FIG. 5).

Cells were seeded into 96-well ultra-low cluster plate (Costar, #7007) in 200 μL complete media and treated at day 2 with DMSO, Staurosporin, or BRD9 degrader, Compound 1, at indicated doses (FIG. 2C). Media and compounds were changed every 5 d and cell colonies were imaged at day 14.

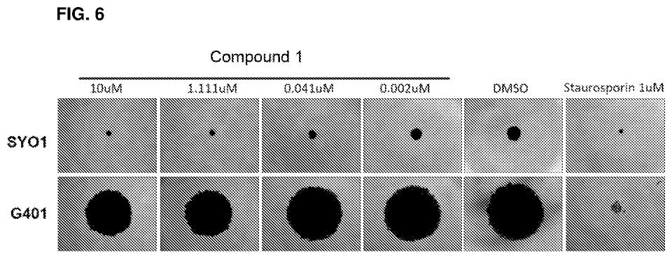

Results: As shown in FIGS. 4, 5, and 6, treatment of synovial sarcoma cell lines (SYO1, Yamato, HS-SY-II, and ASKA) with a BRD9 inhibitor, Compound 2, or a BRD9 degrader, Compound 1, results in inhibition of the growth of the cells, but does not result in inhibition of the growth of non-synovial control cancer cell lines (293T, A549, G401).

### Example 3—Selective Inhibition of Growth of Synovial Cell Lines by BRD9 Degraders and BRD9 Binders

The following example demonstrates that BRD9 degraders and binders selectively inhibit growth of synovial sarcoma cells.

Procedure: Cells were seeded into 6-well or 12-well plates and were treated daily with a BRD9 degrader (Compound 1), a bromo-domain BRD9 binder (Compound 2), E3 ligase binder (lenalidomide), DMSO, or staurosporin (positive control for cell killing), at indicated concentrations. The number of cells was optimized for each cell line. Growth media was refreshed every 5 days. By day 14, medium was removed, cells were washed with PBS, and stained using 500 μL of 0.005% (w/v) crystal violet solution in 25% (v/v) methanol for at least 1 hour at room temperature. Subsequently plates were scanned on an Odyssey CLx Imaging system.

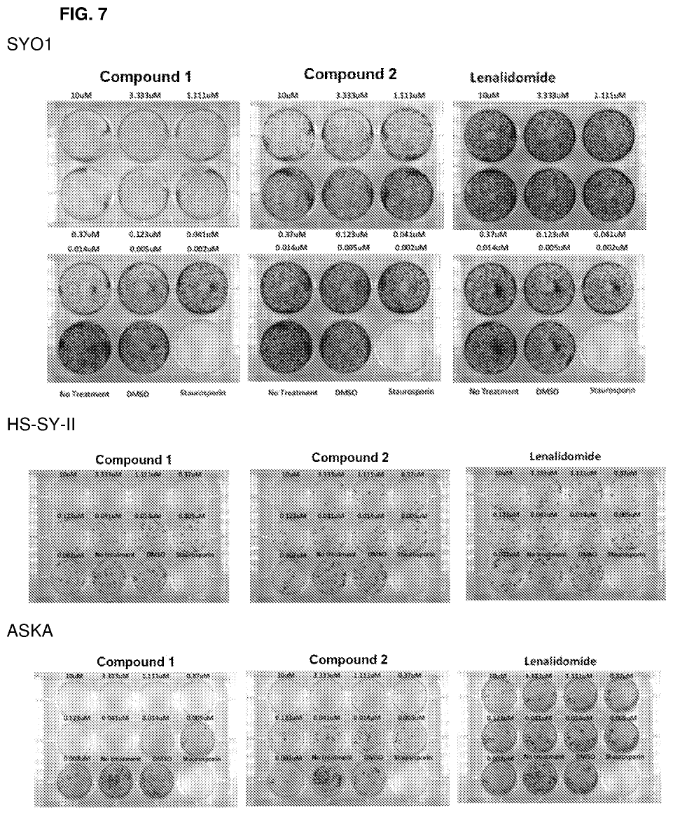

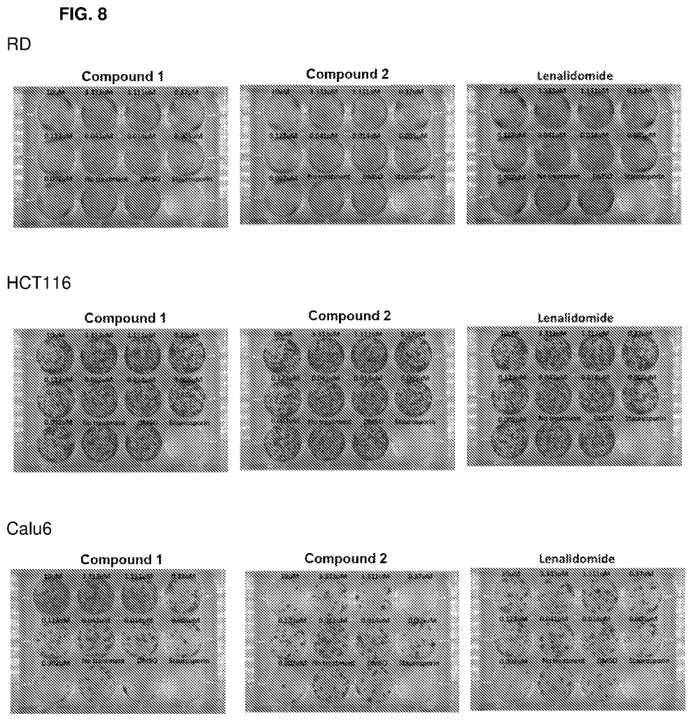

Results: As shown in FIGS. 7 and 8, treatment of synovial sarcoma cell lines (SYO1, HS-SY-II, and ASKA) with Compound 1 or Compound 2 resulted in inhibition of the growth of the cells but did not result in inhibition of the growth of non-synovial control cancer cell lines (RD, HCT116, and Calu6). Overall, Compound 1 showed most significant growth inhibition in all synovial cell lines.

### Example 4—Inhibition of Cell Growth in Synovial Sarcoma Cells

The following example shows that BRD9 degraders inhibit cell growth and induce apoptosis in synovial sarcoma cells.

Procedure: SYO1 cells were treated for 8 or 13 days with DMSO, a BRD9 degrader (Compound 1) at 200 nM or 1 μM, or an E3 ligase binder (lenalidomide) at 200 nM. Compounds were refreshed every 5 days. Cell cycle analysis was performed using the Click-iT™ Plus EdU Flow Cytometry Assay (Invitrogen). The apoptosis assay was performed using the Annexin V-FITC Apoptosis Detection Kit (Sigma A9210). Assays were performed according to the manufacturer's protocol.

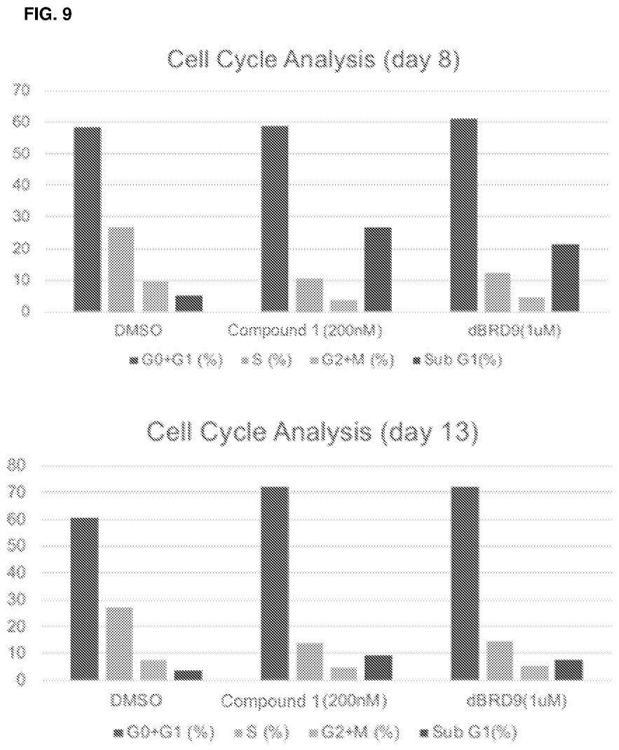

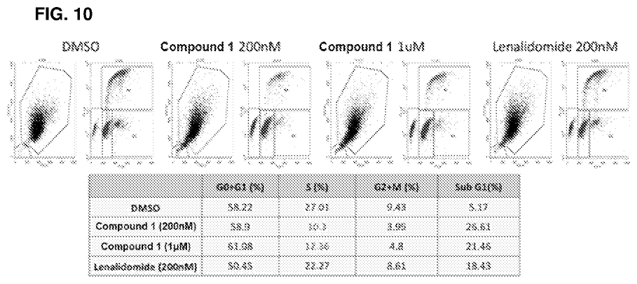

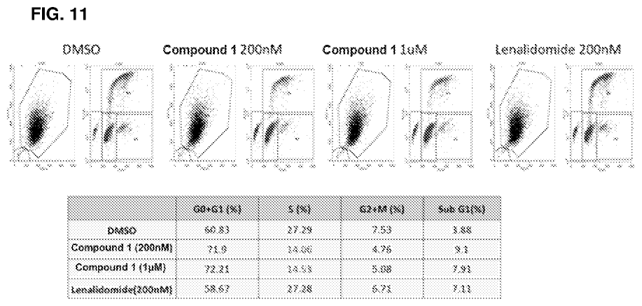

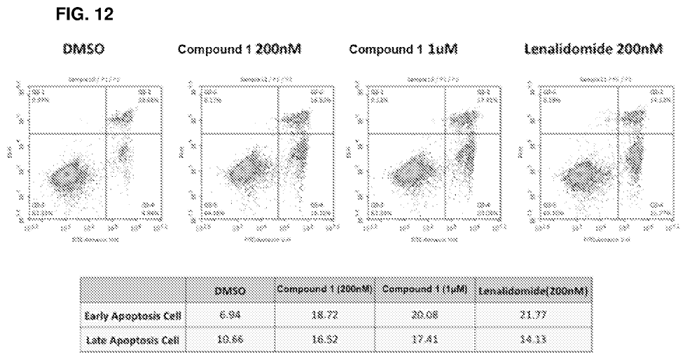

Results: As shown in FIGS. 9-12, treatment with Compound 1 for 8 or 13 days resulted in reduced numbers of cells in the S-phase of the cell cycle as compared to DMSO and lenalidomide. Treatment with Compound 1 for 8 days also resulted in increased numbers of early- and late-apoptotic cells as compared to DMSO controls.

### Example 5—Composition for SS18-SSX1-BAF

The following example shows the identification of BRD9 as a component of SS18-SSX containing BAF complexes.

Procedure: A stable 293T cell line expressing HA-SS18SSX1 was generated using lentiviral integration. SS18-SSX1 containing BAF complexes were subject to affinity purification and subsequent mass spectrometry analysis revealed SS18-SSX1 interacting proteins.

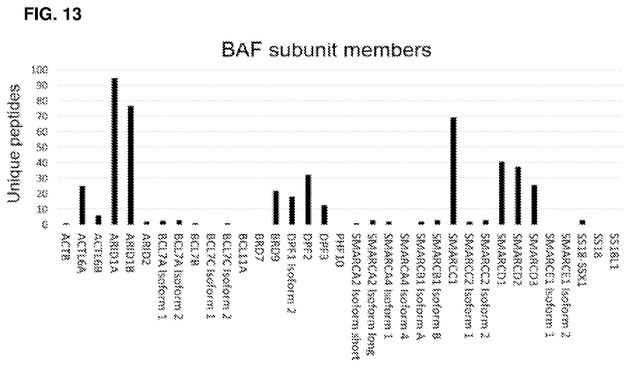

Results: As shown in FIG. 13, BAF complexes including the SS18-SSX fusion protein also included BRD9. More than 5 unique peptides were identified for ARID1A (95 peptides), ARID1B (77 peptides), SMARCC1 (69 peptides), SMARCD1 (41 peptides), SMARCD2 (37 peptides), DPF2 (32 peptides), SMARCD3 (26 peptides), ACTL6A (25 peptides), BRD9 (22 peptides), DPF1 Isoform 2 (18 peptides), DPF3 (13 peptides), and ACTL6B (6 peptides).

### Example 6—Preparation of 4-[6-(azetidin-1-yl)-2-methyl-1-oxo-2,7-naphthyridin-4-yl]-2,6-dimethoxybenzaldehyde (Intermediate H)

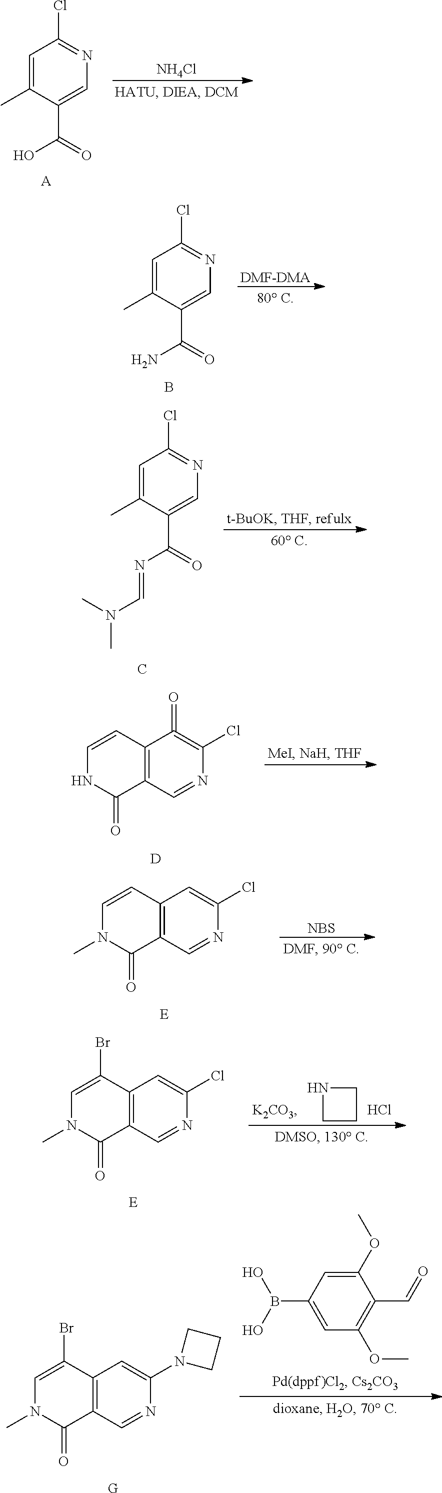

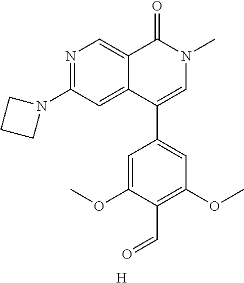

### Step 1: Preparation of 6-chloro-4-methylpyridine-3-carboxamide (Intermediate B)

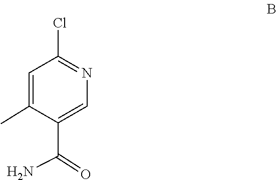

To a stirred mixture of 6-chloro-4-methylpyridine-3-carboxylic acid (20.00 g, 116.564 mmol, 1.00 equiv) and NH₄Cl (62.35 g, 1.17 mol, 10.00 equiv) in dichloromethane (DCM; 400 mL) was added DIEA (22.60 g, 174.846 mmol, 3.00 equiv). After stirring for 5 minutes, HATU (66.48 g, 174.846 mmol, 1.50 equiv) was added in portions. The resulting mixture was stirred for 3 hours at room temperature. The resulting mixture was concentrated under reduced pressure, and the residue was purified by silica gel column chromatography, eluted with petroleum ether/ethyl acetate (PE/EtOAc) from 1/1 to 3/2 to afford 6-chloro-4-methylpyridine-3-carboxamide (18.30 g, 61.3%) as a yellow solid. LCMS (ESI) m/z: [M+H]⁺=171.

### Step 2: Preparation of 6-chloro-N-[(1E)-(dimethylamino) methylidene]-4-methylpyridine-3-carboxamide (Intermediate C)

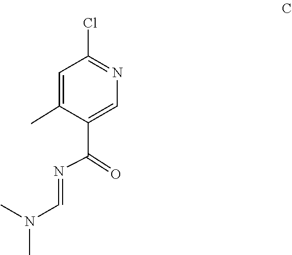

To a stirred mixture of 6-chloro-4-methylpyridine-3-carboxamide (18.30 g, 107.268 mmol, 1.00 equiv) and in 2-methyltetrahydrofuran (100 mL) was added DMF-DMA (19.17 g, 160.903 mmol, 1.50 equiv) at 80° C. under nitrogen atmosphere, and stirred for additional 1 hour. Then the mixture was cooled and concentrated to afford 6-chloro-N-[(1E)-(dimethylamino)methylidene]-4-methylpyridine-3-carboxamide (26.3 g, 91.3%) as a yellow crude solid, which was used directly without further purification. LCMS (ESI) m/z: [M+H]⁺=226.

### Step 3: Preparation of 6-chloro-2H-2,7-naphthyridin-1-one (Intermediate D)

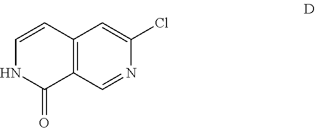 [SMILES: O=c1[nH]ccc2cc(Cl)ncc12]

To a stirred mixture of 6-chloro-N-[(1E)-(dimethylamino) methylidene]-4-methylpyridine-3-carboxamide (26.30 g) in THF (170.00 mL) was added t-BuOK (174.00 mL, 1 mol/L in THF). The resulting solution was stirred at 60° C. under nitrogen atmosphere for 30 minutes. Then the mixture was cooled and concentrated under reduced pressure. The crude solid was washed with saturated NaHCO₃ solution (100 mL) and collected to give 6-chloro-2H-2,7-naphthyridin-1-one (14.1 g, 67.0%) as a pink solid, which was used directly without further purification. LCMS (ESI) m/z: [M+H]⁺=181.

### Step 4: Preparation of 6-chloro-2-methyl-2,7-naphthyridin-1-one (Intermediate E)

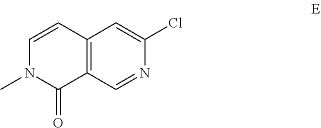

To a stirred mixture of 6-chloro-2H-2, 7-naphthyridin-1-one (14.10 g, 78.077 mmol, 1.00 equiv) in anhydrous THF (280.00 mL) was added NaH (9.37 g, 234.232 mmol, 3.00 equiv, 60%) in portions at 0° C. After 10 minutes, Mel (33.25 g, 234.232 mmol, 3.00 equiv) was added at 0° C., and the mixture was allowed to stir for 10 minutes at 0° C., and then the mixture was allowed to stir for 12 hours at room temperature. The resulting mixture was concentrated under reduced pressure. The crude solid was slurried with water (100 mL), and the solid was filtered and collected to give the 6-chloro-2-methyl-2,7-naphthyridin-1-one (14.6 g, 94.1%) as a yellow solid, which was used directly without further purification. LCMS (ESI) m/z: [M+H]⁺=195.

### Step 5: Preparation of 4-bromo-6-chloro-2-methyl-2,7-naphthyridin-1-one (Intermediate F)

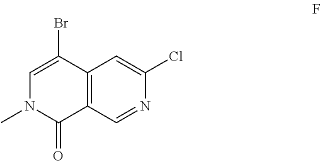

To a stirred mixture of 6-chloro-2-methyl-2,7-naphthyridin-1-one (8.00 g, 41.106 mmol, 1.00 equiv) in DMF (160.00 mL) was added NBS (8.78 g, 49.327 mmol, 1.20 equiv), and the resulting mixture was stirred for 2 hours at 90° C. The reaction mixture was cooled and diluted with DCM (150 mL) and washed with water (3×100 mL). The organic layers were dried and concentrated. Then the residue was slurried with EtOAc (20 mL), and the slurry was filtered. The filter cake was washed with EtOAc (20 mL) to give 4-bromo-6-chloro-2-methyl-2,7-naphthyridin-1-one (6.32 g, 55.7%) as a white solid, which was used directly without further purification. LCMS (ESI) m/z: [M+H]⁺=273.

### Step 6: Preparation of 6-(azetidin-1-yl)-4-bromo-2-methyl-2, 7-naphthyridin-1-one (Intermediate G)

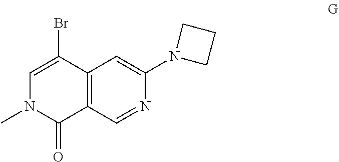 [SMILES: Cn1cc(Br)c2cc(N3CCC3)ncc2c1=O]

To a solution of 4-bromo-6-chloro-2-methyl-2,7-naphthyridin-1-one (5.00 g, 18.281 mmol, 1.00 equiv) and azetidine hydrochloride (3.2 g, 54.843 mmol, 3 equiv) in DMSO (50.00 mL) was added K₂CO₃ (12.6 g, 91.404 mmol, 5 equiv). The resulting solution was stirred at 130° C. for 2 hours. The resulting mixture was cooled and diluted with water (100 mL), and then extracted with EtOAc (3×100 mL). The combined organic layers were washed with saturated NaCl solution (3×50 mL), dried over anhydrous Na₂SO₄, and concentrated under reduced pressure to afford 6-(azetidin-1-yl)-4-bromo-2-methyl-2,7-naphthyridin-1-one (3.7 g, 68.8%) as a grey solid, which was used directly without further purification. LCMS (ESI) m/z: [M+H]⁺=294.

### Step 7: Preparation of 4-[6-(azetidin-1-yl)-2-methyl-1-oxo-2,7-naphthyridin-4-yl]-2,6-dimethoxybenzaldehyde (Intermediate H)

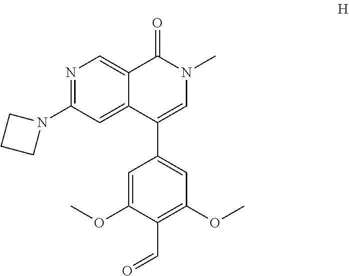 [SMILES: COc1cc(-c2cn(C)c(=O)c3cnc(N4CCC4)cc23)cc(OC)c1C=O]

To a solution of 6-(azetidin-1-yl)-4-bromo-2-methyl-2,7-naphthyridin-1-one (1.42 g, 4.827 mmol, 1.00 equiv) and 4-formyl-3,5-dimethoxyphenylboronic acid (1.52 g, 7.241 mmol, 1.5 equiv) in dioxane (16.00 mL) and H₂O (4.00 mL) was added Pd(dppf)Cl₂ (353.2 mg, 0.483 mmol, 0.1 equiv) and Cs₂CO₃ (3.15 g, 9.655 mmol, 2 equiv), and the resulting solution was stirred at 70° C. for 2 hours. The resulting mixture was cooled and concentrated under reduced pressure. The residue was slurried with water (30 mL) and filtered, and the filter cake was collected. This solid was further slurried with MeOH (30 mL) and filtered. The solid was collected to afford 4-[6-(azetidin-1-yl)-2-methyl-1-oxo-2,7-naphthyridin-4-yl]-2,6-dimethoxybenzaldehyde (1.42 g, 77.5%) as a grey solid. LCMS (ESI) m/z: [M+H]⁺=380.

### Example 7—Preparation of 3-[1-oxo-6-[7-(piperidin-4-ylmethyl)-2,7-diazaspiro[3.5]nonan-2-yl]-3H-isoindol-2-yl]piperidine-2,6-dione (Intermediate P)

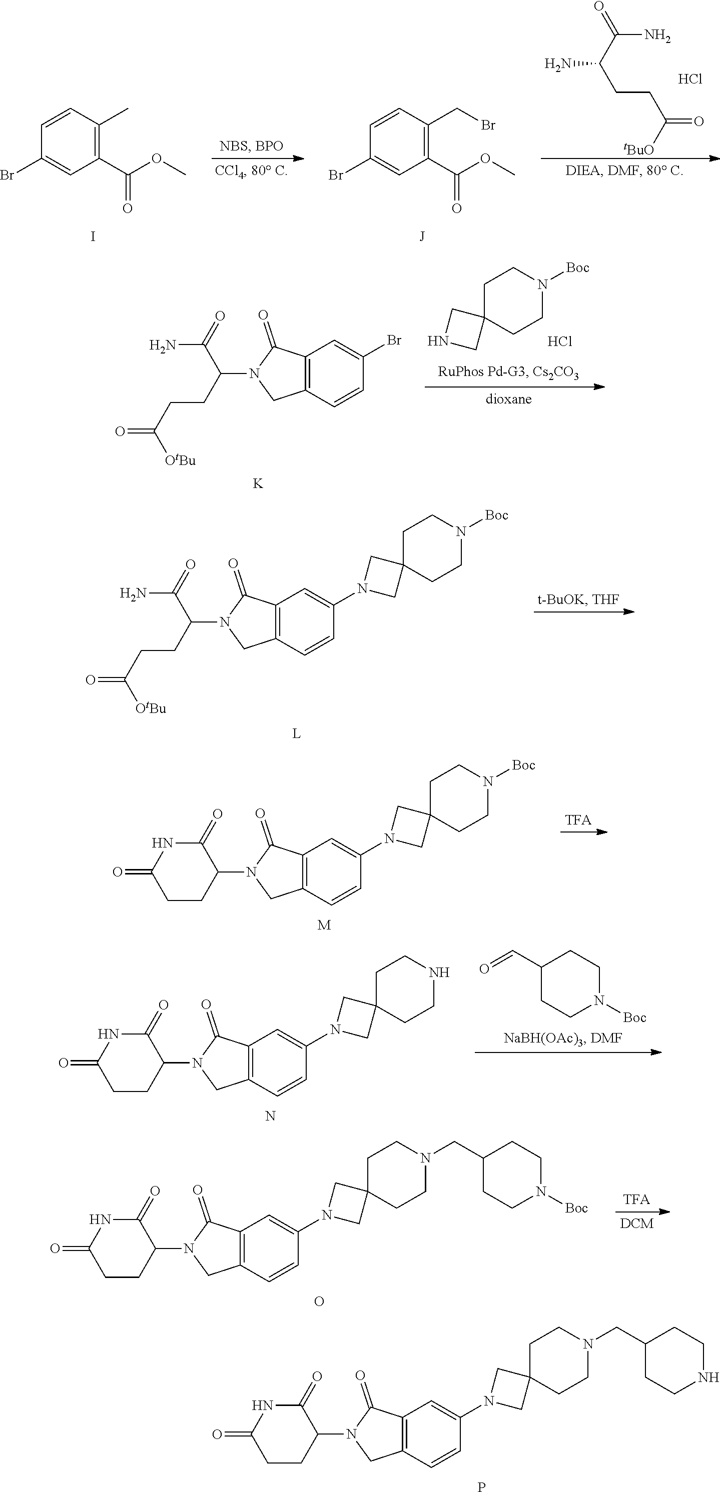

### Step 1: Preparation of methyl 5-bromo-2-(bromomethyl)benzoate (Intermediate J)

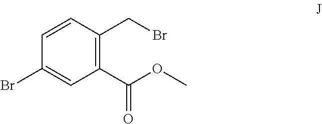

To a stirred mixture of methyl 5-bromo-2-methylbenzoate (50.00 g, 218.271 mmol, 1.00 equiv) in CCl₄ (500.00 mL) was added NBS (38.85 g, 218.271 mmol, 1.00 equiv) and BPO (5.59 g, 21.827 mmol, 0.10 equiv). After stirring for overnight at 80° C., the mixture was purified by silica gel column chromatography, eluted with PE/EtOAc (50:1) to afford methyl 5-bromo-2-(bromomethyl)benzoate (67 g, 74.75%) as a yellow oil. LCMS (ESI) m/z: [M+H]⁺=307.

### Step 2: Preparation of tert-butyl 4-(6-bromo-1-oxo-3H-isoindol-2-yl)-4-carbamoylbutanoate (Intermediate K)

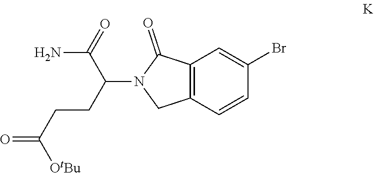 [SMILES: CC(C)(C)OC(=O)CCC(C(N)=O)N1Cc2ccc(Br)cc2C1=O]

To a stirred mixture of methyl 5-bromo-2-(bromomethyl)benzoate (67.00 g, 217.554 mmol, 1.00 equiv) and tert-butyl (4S)-4-amino-4-carbamoylbutanoate hydrochloride (62.32 g, 261.070 mmol, 1.20 equiv) in DMF (100.00 mL) was added DIEA (112.47 g, 870.217 mmol, 4 equiv). After stirring for overnight at 80° C., the mixture was concentrated under reduced pressure. The residue was added water (200 mL) and stirred for 1 h at room temperature. The resulting mixture was filtered, the filter cake was added water (100 mL) and stirred. The precipitated solids were collected by filtration and washed with water (3×30 mL). This resulted in tert-butyl 4-(6-bromo-1-oxo-3H-isoindol-2-yl)-4-carbamoylbutanoate (55 g, 60.46%) as an off-white solid. LCMS (ESI) m/z: [M+H]⁺=397.

### Step 3: Preparation of tert-butyl 2-[2-[4-(tert-butoxy)-1-carbamoyl-4-oxobutyl]-3-oxo-1H-isoindol-5-yl]-2,7-diazaspiro[3.5]nonane-7-carboxylate (Intermediate L)

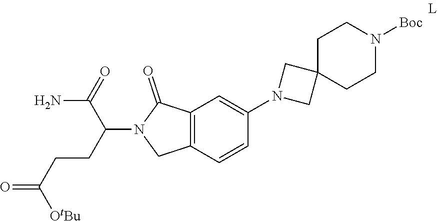 [SMILES: CC(C)(C)OC(=O)CCC(C(N)=O)N1Cc2ccc(N3CC4(CCN(C(=O)OC(C)(C)C)CC4)C3)cc2C1=O]

To a stirred solution of tert-butyl 4-(6-bromo-1-oxo-3H-isoindol-2-yl)-4-carbamoylbutanoate (10.00 g, 25.172 mmol, 1.00 equiv) and tert-butyl 2,7-diazaspiro[3.5]nonane-7-carboxylate hydrochloride (8.60 g, 32.723 mmol, 1.30 equiv) in dioxane (200.00 mL) was added Cs₂CO₃ (24.60 g, 75.516 mmol, 3.00 equiv) and RuPhos Palladacycle Gen.3 (2.11 g, 2.517 mmol, 0.10 equiv). After stirring for overnight at 100° C. under nitrogen atmosphere, the resulting mixture was filtered while hot, and the filter cake was washed with 1,4-dioxane (3×50 mL). The filtrate was concentrated under reduced pressure to afford tert-butyl 2-[2-[4-(tert-butoxy)-1-carbamoyl-4-oxobutyl]-3-oxo-1H-isoindol-5-yl]-2,7-diazaspiro [3.5]nonane-7-carboxylate (21 g, crude) as a black solid. LCMS (ESI) m/z: [M+H]⁺=543.

### Step 4: Preparation of tert-butyl 2-[2-(2,6-dioxopiperidin-3-yl)-3-oxo-1H-isoindol-5-yl]-2,7-diazaspiro[3.5]nonane-7-carboxylate (Intermediate M)

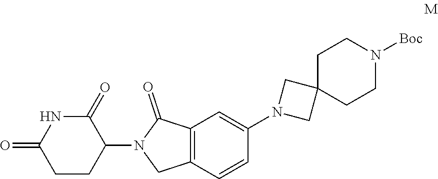 [SMILES: CC(C)(C)OC(=O)N1CCC2(CC1)CN(c1ccc3c(c1)C(=O)N(C1CCC(=O)NC1=O)C3)C2]

To a stirred mixture of tert-butyl 2-[2-[(1S)-4-(tert-butoxy)-1-carbamoyl-4-oxobutyl]-3-oxo-1H-isoindol-5-yl]-2,7-diazaspiro[3.5]nonane-7-carboxylate (13.68 g, 25.208 mmol, 1.00 equiv) in THF (100.00 mL) was added t-BuOK in THF (25.00 mL, 25.000 mmol, 0.99 equiv). The resulting mixture was stirred for 2 hours at room temperature. The mixture was acidified to pH 6 with 1 M HCl (aq.) and then neutralized to pH 7 with saturated NaHCO₃(aq.). The resulting mixture was extracted with EtOAc (3×200 mL). The combined organic layers were concentrated under reduced pressure. This resulted in tert-butyl 2-[2-(2,6-dioxopiperidin-3-yl)-3-oxo-1H-isoindol-5-yl]-2,7-diazaspiro[3.5]nonane-7-carboxylate (7.8 g, 59.43%) as a yellow solid. LCMS (ESI) m/z: [M+H]⁺=469.

### Step 5: Preparation of 3-(6-[2,7-diazaspiro[3.5]nonan-2-yl]-1-oxo-3H-isoindol-2-yl)piperidine-2,6-dione (Intermediate N)

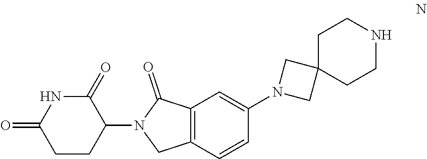 [SMILES: O=C1CCC(N2Cc3ccc(N4CC5(CCNCC5)C4)cc3C2=O)C(=O)N1]

To a stirred mixture of tert-butyl 2-[2-(2,6-dioxopiperidin-3-yl)-3-oxo-1H-isoindol-5-yl]-2,7-diazaspiro[3.5]nonane-7-carboxylate (7.80 g, 16.647 mmol, 1.00 equiv) in DCM (10.00 mL) was added trifluoroacetic acid (TFA; 5.00 mL). After stirring for 2 hours at room temperature, the resulting mixture was concentrated under vacuum. This resulted in 3-(6-[2,7-diazaspiro[3.5]nonan-2-yl]-1-oxo-3H-isoindol-2-yl) piperidine-2,6-dione (6 g, 92.93%) as a light yellow solid. LCMS (ESI) m/z: [M+H]⁺=369.

### Step 6: Preparation of tert-butyl 4-([2-[2-(2,6-dioxopiperidin-3-yl)-3-oxo-1H-isoindol-5-yl]-2,7-diazaspiro[3.5]nonan-7-yl]methyl)piperidine-1-carboxylate (Intermediate O)

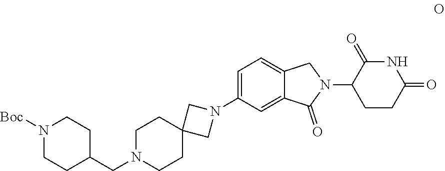 [SMILES: CC(C)(C)OC(=O)N1CCC(CN2CCC3(CC2)CN(c2ccc4c(c2)C(=O)N(C2CCC(=O)NC2=O)C4)C3)CC1]

To a stirred solution of 3-(6-[2,7-diazaspiro[3.5]nonan-2-yl]-1-oxo-3H-isoindol-2-yl) piperidine-2,6-dione (4.00 g, 8.685 mmol, 1.00 equiv, 80%) and tert-butyl 4-formylpiperidine-1-carboxylate (1.48 g, 6.939 mmol, 0.80 equiv) in DMF (20.00 mL) was added NaBH(OAc)₃ (3.68 g, 17.363 mmol, 2.00 equiv) at room temperature. The resulting mixture was stirred for 2 hours at room temperature. The reaction was quenched with water at room temperature. The resulting mixture was purified by reverse flash chromatography with the following conditions (column, C18 silica gel; mobile phase, CH₃CN in water (0.1% FA), 0 to 100% gradient in 40 minutes; detector, UV 254 nm). This resulted in tert-butyl 4-([2-[2-(2,6-dioxopiperidin-3-yl)-3-oxo-1H-isoindol-5-yl]-2,7-diazaspiro[3.5]nonan-7-yl]methyl)piperidine-1-carboxylate (2.8 g, 51.29%) as a dark yellow solid. LCMS (ESI) m/z: [M+H]⁺=566.

### Step 7: Preparation of 3-[1-oxo-6-[7-(piperidin-4-ylmethyl)-2,7-diazaspiro[3.5]nonan-2-yl]-3H-isoindol-2-yl]piperidine-2,6-dione (Intermediate P)

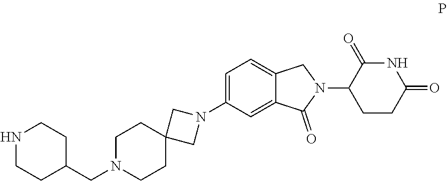 [SMILES: O=C1CCC(N2Cc3ccc(N4CC5(CCN(CC6CCNCC6)CC5)C4)cc3C2=O)C(=O)N1]

To a stirred mixture of tert-butyl 4-([2-[2-(2,6-dioxopiperidin-3-yl)-3-oxo-1H-isoindol-5-yl]-2,7-diazaspiro[3.5]nonan-7-yl]methyl)piperidine-1-carboxylate (2.80 g, 4.949 mmol, 1.00 equiv) in DCM (5.00 mL) was added TFA (2.00 mL). The mixture was stirred for 2 hours at room temperature. The resulting mixture was concentrated under reduced pressure to afford 3-[1-oxo-6-[7-(piperidin-4-ylmethyl)-2,7-diazaspiro[3.5]nonan-2-yl]-3H-isoindol-2-yl]piperidine-2,6-dione (3.9 g, crude) as a yellow solid. LCMS (ESI) m/z: [M+H]⁺=466.

### Example 8—Preparation of 3-[6-(7-[[1-([4-[6-(azetidin-1-yl)-2-methyl-1-oxo-2,7-naphthyridin-4-yl]-2,6-dimethox yphenyl]methyl)piperidin-4-yl]methyl]-2,7-diazaspiro[3.5]nonan-2-yl)-1-oxo-3H-isoindol-2-yl]piperidine-2,6-dione TFA Salt (Compound D1 TFA Salt)

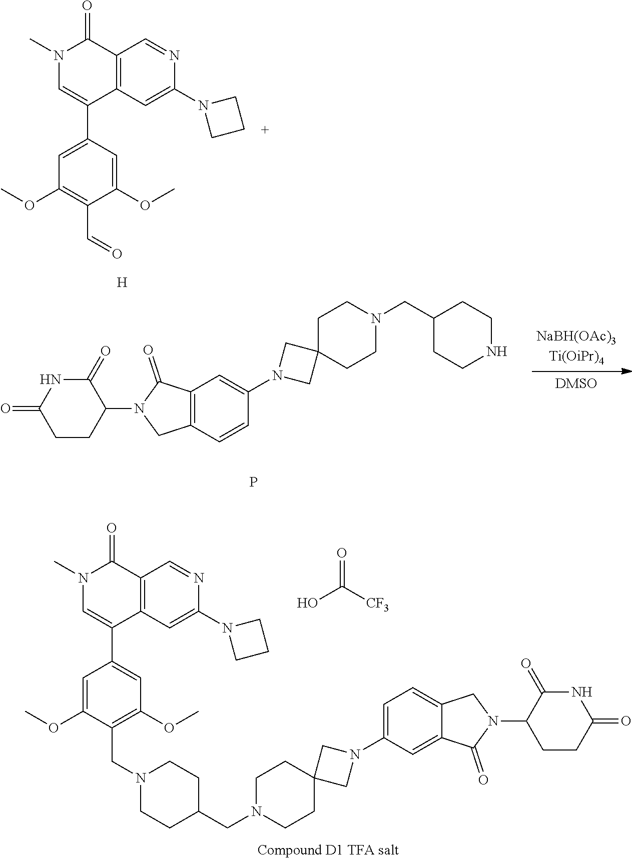

A solution of 3-[1-oxo-6-[7-(piperidin-4-ylmethyl)-2,7-diazaspiro[3.5]nonan-2-yl]-3H-isoindol-2-yl]piperidine-2,6-dione (4.5 g, 10.52 mmol, 1.00 equiv) and 4-[6-(azetidin-1-yl)-2-methyl-1-oxo-2,7-naphthyridin-4-yl]-2,6-dimethoxybenzaldehyde (4.0 g, 10.52 mmol, 1.00 equiv) and titanium (IV) isopropoxide (3.0 g, 10.52 mmol, 1.00 equiv) in DMSO (100 mL) was stirred at room temperature for 3 hours. Then NaBH(OAc)₃ (8.92 g, 42.08 mmol, 4.00 equiv) was added in batches to the above resulting solution, and the resulting mixture was stirred at room temperature overnight. The reaction was quenched by the addition of water (30 mL), and then the solution was filtered. The filter cake was wash by water and acetonitrile. Then the filtrate was concentrated in vacuo. The crude product was purified by reverse phase flash chromatography with the following conditions (Column: AQ C₁₈ Column, 50×250 mm 10 um; Mobile Phase A: Water (TFA 0.1%), Mobile Phase B: ACN; Flow rate: 100 mL/minute; Gradient: 5 B to 25 B in 35 minutes; 254/220 nm). Pure fractions were evaporated to dryness to afford 3-[6-(7-[[1-([4-[6-(azetidin-1-yl)-2-methyl-1-oxo-2,7-naphthyridin-4-yl]-2,6-dimethoxyphenyl]methyl)piperidin-4-yl]methyl]-2,7-diazaspiro[3.5]nonan-2-yl)-1-oxo-3H-isoindol-2-yl]piperidine-2,6-dione TFA salt (3.2 g, 32.3%) as a white solid. ¹H NMR (400 MHZ, DMSO-d6) δ 10.96 (s, 1H), 9.01 (s, 1H), 7.59 (s, 1H), 7.36 (d, J=8.0 Hz, 1H), 6.72 (s, 2H), 6.68 (d, J=8.1 Hz, 2H), 6.20 (s, 1H), 5.07 (dd, J=13.3, 5.1 Hz, 1H), 4.35-4.13 (m, 2H), 4.06-3.95 (m, 4H), 3.80 (s, 6H), 3.57 (s, 4H), 3.47 (s, 5H), 2.97-2.75 (m, 3H), 2.70-2.55 (m, 1H), 2.44-2.16 (m, 7H), 2.13-1.88 (m, 5H), 1.80-1.67 (m, 4H), 1.61 (d, J=12.4 Hz, 2H), 1.53-1.33 (m, 1H), 1.13-0.94 (m, 2H). LCMS (ESI) m/z: [M+H]⁺=829.55.

Enantiomers of compound D1 were separated by supercritical fluid chromatography on chiral support to produce compound S-D1 and compound R-D1.

Compound D1 is of the following structure:

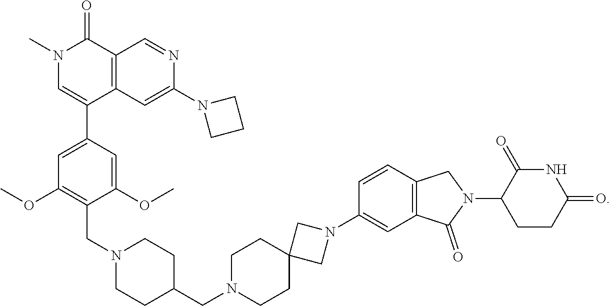 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N(C2CCC(=O)NC2=O)C4)C3)CC1]

Compound S-D1 is of the following structure:

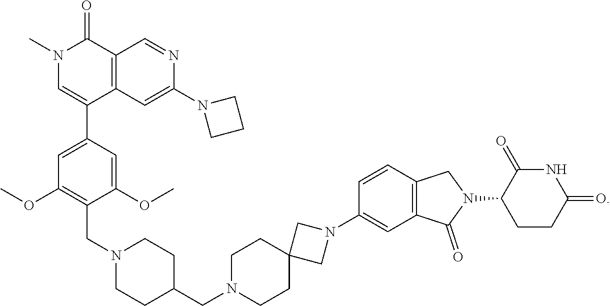 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N([C@H]2CCC(=O)NC2=O)C4)C3)CC1]

Compound R-D1 is of the following structure:

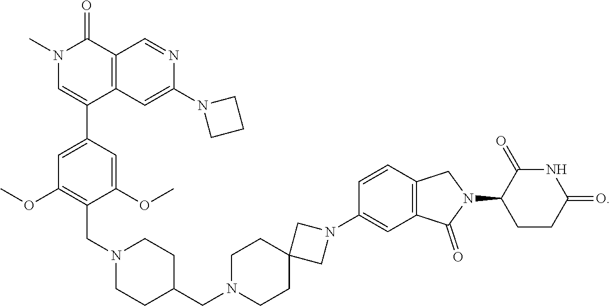 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N([C@@H]2CCC(=O)NC2=O)C4)C3)CC1]

### Example 9—SYO1 BRD9 NanoLuc Degradation Assay

This example demonstrates the ability of the compounds of the disclosure to degrade a Nanoluciferase-BRD9 fusion protein in a cell-based degradation assay.

Procedure: A stable SYO-1 cell line expressing 3×FLAG-NLuc-BRD9 was generated. On day 0 cells were seeded in 30 μL media into each well of 384-well cell culture plates. The seeding density was 8000 cells/well. On day 1, cells were treated with 30 nL DMSO or 30 nL of 3-fold serially DMSO-diluted compounds (10 points in duplicates with 1 μM as final top dose). Subsequently plates were incubated for 6 hours in a standard tissue culture incubator and equilibrated at room temperature for 15 minutes. Nanoluciferase activity was measured by adding 15 μL of freshly prepared Nano-Glo Luciferase Assay Reagent (Promega N1130), shaking the plates for 10 minutes and reading the bioluminescence using an EnVision reader.

Results: The Inhibition % was calculated using the following formula: % Inhibition=100×(Lum_{HC}−Lum_{Sample})/(Lum_{HC}−Lum_{LC}). DMSO treated cells are employed as High Control (HC) and 1 μM of a known BRD9 degrader standard treated cells are employed as Low Control (LC). The data was fit to a four parameter, non-linear curve fit to calculate IC₅₀ (UM) values as shown in Table 2. As shown by the results in Table 2, a number of compounds of the present disclosure exhibit an IC₅₀ value of <1 μM for the degradation of BRD9, indicating their use as compounds for reducing the levels and/or activity of BRD9 and their potential for treating BRD9-related disorders.

TABLE 2
SYO1 BRD9-NanoLuc Degradation

| Compound No. | SYO1 BRD9-NanoLuc degradation IC₅₀ (nM) |
|---|---|
| D1 | 0.13 |
| D2 | 0.18 |

### Example 10—Degradation of BRD9 Inhibits the Growth of Synovial Sarcoma Tumor In Vivo

Method: NOD SCID mice (Beijing Anikeeper Biotech, Beijing) were inoculated subcutaneously on the right flank with the single cell suspension of SYO-1 human biphasic synovial sarcoma tumor cells (5×106) in 100 μL Dulbecco's Modified Eagle Medium (DMEM) with 10% fetal bovine serum (FBS). The mice were randomized into either control group [10% dimethyl sulfoxide (DMSO), 40% polyethylene glycol (PEG400) and 50% water], or treatment group D1 when the mean tumor size reached about 117 mm³. Mice were dosed daily through intraperitoneal (i.p.) route over the course of 3 weeks. All dose volumes were adjusted by body weights in terms of mg/kg.

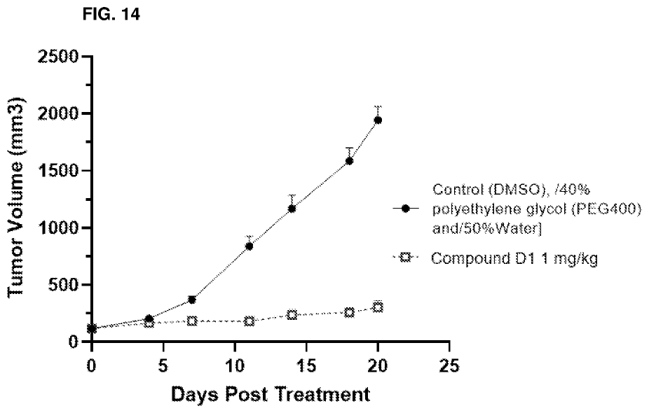

Results: As shown in FIG. 14, treatment with compound D1 at 1 mg/kg had led to tumor growth inhibition. All treatments were well tolerated based on % body weight change observed.

### Example 11—Compound D1 Causes Degradation of BRD9 in Synovial Sarcoma Tumor In Vivo

Method: Mice were treated with D1, 1 mg/kg, i.p. for 4 weeks. Mice were then euthanized, and tumors were collected at 8 hours, 72 hours, and 168 hours post last dose. Tumors were lysed with 1x RIPA lysis buffer (Boston BioProducts, BP-115D) with protease and phosphatase protein inhibitor (Roche Applied Science #04906837001 & 05892791001). Equal amounts of lysate (30 μg) were loaded in in 4-12% Bis-Tris Midi Protein Gels in 1×MOPS buffer; samples ran at 120 V for 120 minutes. Protein was transferred to membrane with TransBlot at 250 mA for 150 minutes, and then membranes were blocked with Odyssey blocking buffer for 1 hour at room temperature. Membranes were hybridized overnight in cold room with primary antibodies. Images acquired using Li-COR imaging system (Li-COR Biotechnology, Lincoln, Nebraska).

Table 3 shows detection antibody information.

TABLE 3

| Antibody | Vendor | Cat# | Species | Dilution |
|---|---|---|---|---|
| BRD9 | Bethyl, (Montgomery, TX) | A303-781A | Rabbit | 1:1000 |
| GAPDH | CST, (Danvers, MA) | 97166 | Mouse | 1:2000 |

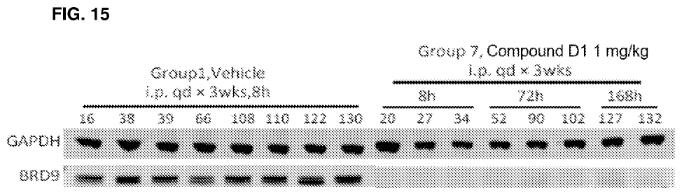

Results: As shown in FIG. 15, treatment with compound D1 at 1 mg/kg led to complete degradation of BRD9 target up to 168 hours after dose.

### Example 12—the Effect of Compounds S-D1 and R-D1 on Synovial Sarcoma Cells

Method. Synoial sarcoma cells were plated in 6-well plate at 500-100 k cells/well and treated with serial concentrations of BRD9 degrader (10 nM top concentration, diluted 1:3) the next day for two time points at 37° C. Cells were then harvested, washed with cold PBS, and frozen in cell pellets at −80° C. Lysates were prepared by resuspending thawed pellets in 1x RIPA Lysis and Extraction buffer (Thermo Fisher, Cat #89900) with 1×Halt™ Protease and Phosphatase Inhibitor Cocktail, EDTA-free (Thermo Fisher, Cat #78441) and 1:1000 dilution Pierce™ Universal Nuclease for Cell Lysis 25 ku (Thermo Fisher, Cat #88700). Lysates were incubated on ice for 10 minutes and then centrifuged in 4° C. at maximum speed (15,000 rpm) for 10 minutes. Samples were then analyzed for total protein using BCA protein quantification assay and diluted to 1 μg/μL with lysis buffer and 1×NuPAGE™ LDS Sample Buffer (4×) (Thermo Fisher, Cat #NP0007) and 1×DTT from 30× stock (Cell Signaling Technologies, Cat #14265S). Samples with 20-25 ug of total protein were loaded into 4-12% Bis-Tris Mini-Gel with 1×MES Running buffer and run at 150V for 45 minutes. Gels were transferred using Trans-Blot® Turbo™ Transfer System (semi-dry) at 25V for 10 minutes (High MW setting) on nitrocellulose blots. Blots were blocked in 5% milk in TBST for 1 hour and probed with BRD9 antibody (Bethyl Labs, Cat #A303-781A, 1:750 for SYO1, and Cell Signaling Technologies, Cat #71232S for ASKA) and beta-Actin antibody (Cell Signaling Technologies, Cat #3700, 1:2000) overnight at 4° C. The next day, blots were washed in TBST 3x and probed with 1:5000 IRDye® 680LT Goat anti-Rabbit IgG Secondary Antibody (LICOR, Cat #926-68021) and 1:10000 IRDye® 800CW Goat anti-Mouse IgG Secondary Antibody (LiCOR, Cat #926-32210) in LiCOR Odyssey® Blocking Buffer (TBS) for 1 hour at room temperature. Blots were washed in TBST 3x and scanned at 700 nM and 800 nM wavelength using LiCOR Odyssey® CLx Imaging System. Western blot signal was quantified using same analyses program included in the same machine. BRD9 signal was quantified by normalizing to beta-actin signal and all samples were normalized to DMSO, set as 100% signal.

For the assessment of interconversion between Enantiomer 1 and Enantiomer 2 in cell medium, the following test was performed. Enantiomer 1 and Enantiomer 2 (each was 40 μM in DMSO) was spiked into cell medium (DMEM+Glutamax+10% FBS) at a final concentration of 0.2 μM and incubated at 37° C. and 5% CO₂ in duplicate. At designated time point, aliquot (50 μL) was taken and processed by the addition of 150 μL of acetonitrile containing 0.1% formic acid and internal standard for LC/MS-MS analysis. Peak areas of both Enantiomer 1 and Enantiomer 2 were determined for each sample using a chiral specific analytical method. The results are summarized in Table 5 below.

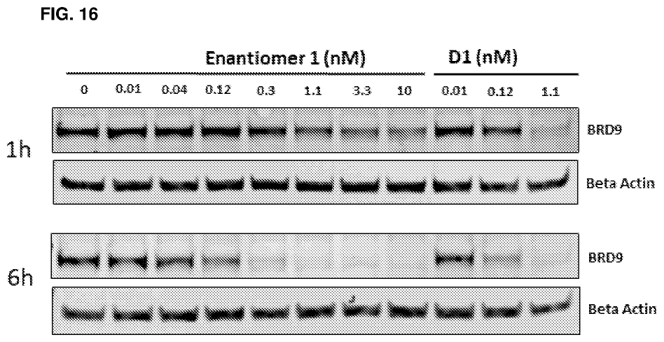

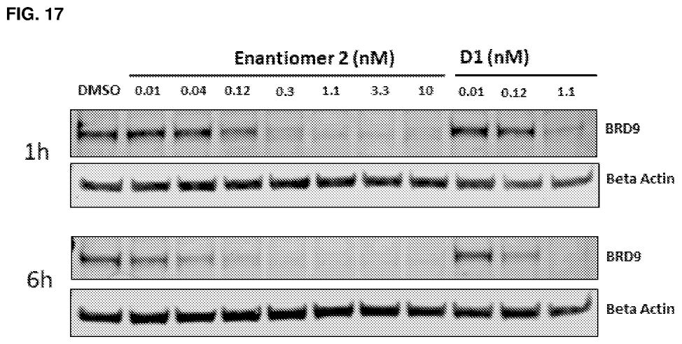

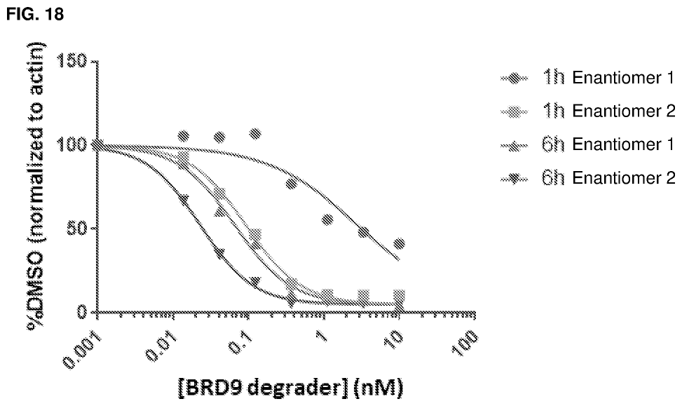

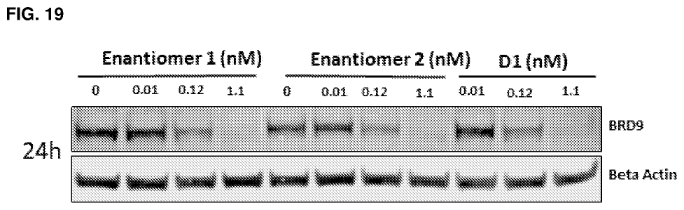

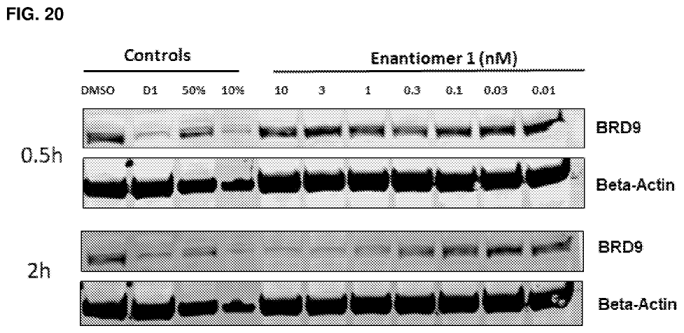

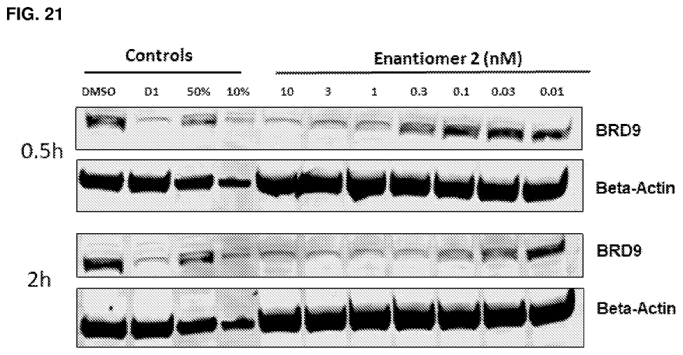

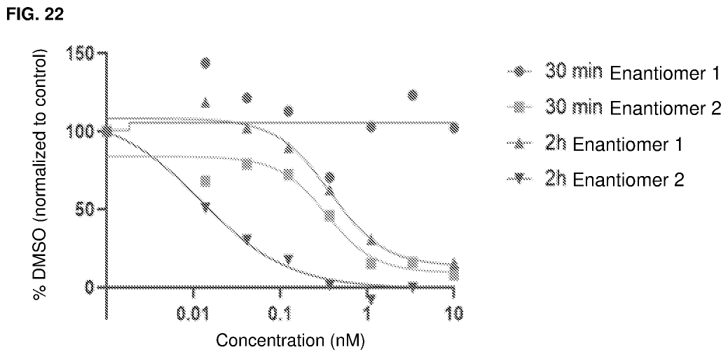

Results. To assess BRD9 degradation activity of two enantiomers, degrader treatment and subsequent western-blot experiments were carried out using two synovial sarcoma cell lines (SYO-1 and ASKA). Significant more potent BRD9 degradation activity was observed with Enantiomer 2, with a fitted DC₅₀ value of 0.092 nM, comparing to 2.8 nM for Enantiomer 1 in SYO-1 with 1 h treatment time (FIGS. 16, 17, and 18 and Table 4). Even more dramatic difference in ASKA cells is evident with a DC₅₀ of 0.34 nM for Enantiomer 2 at 30 minutes, but there is no discernable activity for Enantiomer 1 up to 10 UM at the same time point (FIGS. 20, 21, and 22 and Table 4). The difference is reduced to about 32-fold at 2 h in ASKA, with a fitted DC₅₀ value of 0.38 nM and 0.012 nM for Enantiomer 1 and Enantiomer 2, respectively (Table 4). The difference is further reduced to ca. 3-fold by 6 h in SYO1. Enantiomer 2 works slightly better than its racemic parent compound D1 in degrading BRD9 but overall comparable under the same study conditions (FIGS. 16 and 17). BRD9 degradation activity becomes highly similar for all three compounds at 24 h (FIG. 19). Taking together, Enantiomer 2 is much more potent in degradation endogenous BRD9 protein in two synovial sarcoma cell lines at early time point, whereas Enantiomer 1 is largely inactive or with much reduced degradation potency. However, the difference in potency is diminished over time and largely disappeared by 24 h.

TABLE 4

| Cell | Fitted | Enantiomer | Enantiomer |
|---|---|---|---|
| Line | DC₅₀ (nM) | 1 | 2 |
| ASKA | 0.5 h | >10 | 0.34 |
| SYO-1 | 1 h | 2.8 | 0.092 |
| ASKA | 2 h | 0.38 | 0.012 |
| SYO-1 | 6 h | 0.066 | 0.023 |

Epimerization of the chiral center in thalidomide or other IMiD drugs and their derivatives is reported. The acidic hydrogen in the chiral center can be scrambled under physical or neutral pH conditions. To investigate the chiral stability under cell assay conditions for these degraders, we performed a time course study for Enantiomer 1 and Enantiomer 2 in cell culture medium at 37° C. There is no detectable Enantiomer 2 in Enantiomer 1 samples at time 0 or 0.5 h. But substantial Enantiomer 2 was detected at later time points, accounting for 12% and 30% of the total at 2 h and 6 h, respectively (Table 5). Similarly, Enantiomer 2 is converted to Enantiomer 1 over time and its effective concentration was reduced to 63% at 6h (Table 5). These data indicate that epimerization rate is relatively fast under the cell assay conditions, and suggest that the time-dependent BRD9 degradation activity for Enantiomer 1 is likely due to the converted Enantiomer 2. Overall, these data indicate that Enantiomer 2 is the active enantiomer in degrading BRD9 in cells.

TABLE 5

| | Enantiomer 1 Dosing | | Enantiomer 2 Dosing | |
|---|---|---|---|---|
| | Mean peak | | Mean peak | |
| | area ratio | | area ratio | |
| | of Enantiomer | | of Enantiomer | |
| | 2 over | % | 1 over | % |
| Time | Enantiomer 1 | Enantiomer | Enantiomer 2 | Enantiomer |
| (h) | peak area ratio | 2 | peak area ratio | 2 |
| 0 | 0.0 | 0.0 | 0.01 | 99 |
| 0.5 | 0.0 | 0.0 | 0.06 | 95 |
| 2 | 0.13 | 12 | 0.22 | 82 |
| 6 | 0.43 | 30 | 0.60 | 63 |

### Example 13—The Effect of Compounds S-D1 and R-D1 on Synovial Sacroma Cells

Method. The SYO-1 tumor cells were maintained in vitro as adherent cells in Dulbecco's Modified Eagle Medium (DMEM) supplemented with 10% fetal bovine serum at 37° C. in an atmosphere of 5% CO2 in air. The cells growing in an exponential growth phase were harvested and counted for tumor inoculation. BALB/c Nude mice (Shanghai Lingchang biological science) were inoculated subcutaneously on the right flank with (5×10⁶) in 0.1 mL of phosphate buffered saline (PBS). The treatment—(described in the table below)—was started on day 19 after tumor inoculation, when the average tumor size reached 499 mm³.

TABLE 6
Treatment information

| Antibody | Number of mice |
|---|---|
| Vehicle control, sulfobutylether-β- | 3 |
| cyclodextrin (SBECD), Single | |
| Dose, 10 μL/g | |
| Racemic D1, 0.5 mg/kg i.v., Single | 12 |
| Dose, 10 μL/g (20% SBECD) | |
| Enantiomer 1, 0.25 mg/kg i.v., Single | 18 |
| Dose, 10 μL/g (20% SBECD) | |
| Enantiomer 2, 0.25 mg/kg i.v., Single | 18 |
| Dose, 10 μL/g (20% SBECD) | |
| Enantiomer 2, 1 mg/kg i.v., Single | 18 |
| Dose, 10 μL/g (20% SBECD) | |

Mice were treated with racemic D1, 1 mg/kg, i.p. for 4 weeks, mice were euthanized, and tumors collected 1, 4, 8, 24, 48 and 72-hour post last dose. Tumors were lysed with 1×RIPA lysis buffer (Boston BioProducts, BP-115D) with protease and phosphatase protein inhibitor (Roche Applied Science #04906837001 & 05892791001). Equal amount of lysate (30 ug) were loaded in in 4-12% Bis-Tris Midi Protein Gels in 1×MOPS buffer; samples ran at 120 V for 120 min. Proteins was transferred to membrane (NC) with TransBlot at 250 mA for 150 minute, then membranes were blocked with Odyssey Blocking buffer for 1 hour at room temperature. Membranes were hybridized overnight in cold room with primary antibodies. Images acquired using Li-COR imaging system (Li-COR Biotechnology, Lincoln, Nebraska)

TABLE 7
Detection antibody information

| Antibody | Vendor | Cat# | Species | Dilution |
|---|---|---|---|---|
| BRD9 | Bethyl, (Montgomery, TX) | A303-781A | Rabbit | 1:1000 |
| GAPDH | CST, (Danvers, MA) | 97166 | Mouse | 1:2000 |

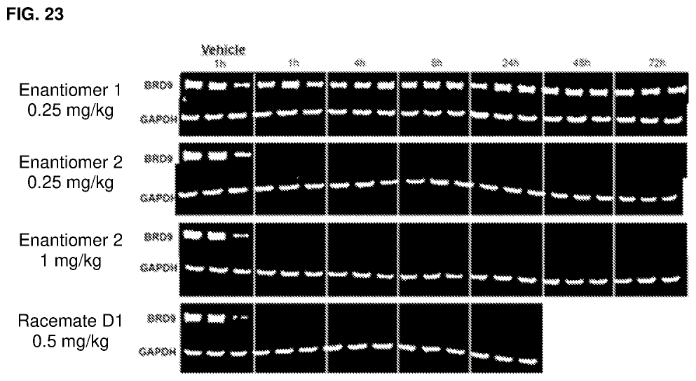

Results. Pharmacodynamic activities of Enantiomer 1, Enantiomer 2, and racemic compound D1 were evaluated in SYO-1 Xenograft model. Enantiomer 2 demonstrated significant activity which was assessed by BRD9 protein level using western blot assay FIG. 23. Enantiomer 2 degraded BRD9 up to 72 hours after a single dose. Enantiomer 1 was inactive and did not degrade BRD9 protein. These results suggested Enantiomer 2 is equipotent to racemic compound D1.

### Example 14—Stereoretentive Preparation of Compound S-D1

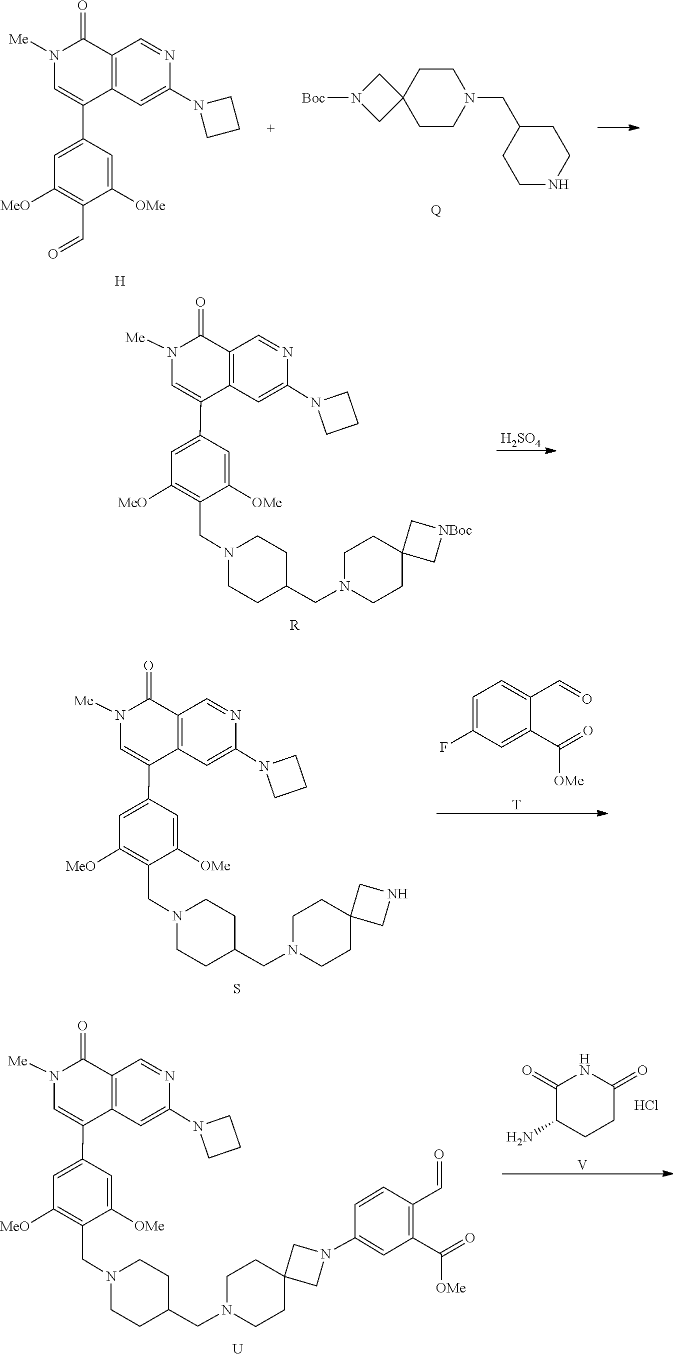

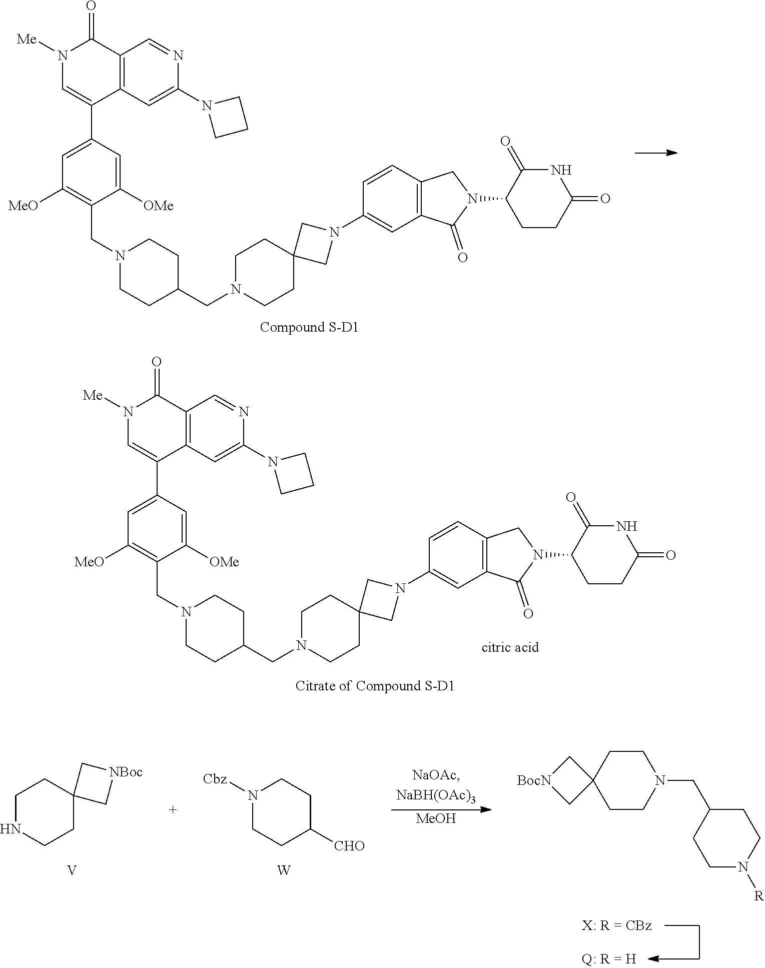

Preparation of Compound X. To V and Win MeOH were added NaOAc (2.0 eq) and sodium triacetoxyborohydride (3.0 eq), and the reaction mixture was stirred. Upon completion of the reaction, the reaction was quenched with water, followed by further dilution with NaOH (7%). The mixture was seeded with compound X. To the reaction mixture was added more NaOH (7%) until the pH of the solution was ˜8-9. The slurry was filtered and washed to obtain compound X in >90% yield.

Hydrogenation of Compound X, Formation of Compound Q. To a mixture of compound X in MeOH was added 1% w/w Pd/C. This mixture was put under 40 psi hydrogen pressure and stirred for 16 h. The reaction mixture was then filtered and washed with MeOH. The resulting solution was concentrated, and then IPAc was added. This mixture was concentrated to remove MeOH, and further IPAc was added. The IPAc solution was then concentrated, and to the mixture was added n-heptane to allow for precipitation of the product. This mixture was cooled and stirred overnight. The solid was filtered and dried to provide Compound Q in ˜75% yield.

Reductive Amination to Compound R: Compound Q and Compound H Coupling. To Compound Q and Compound H in NMP, were added NaOAc (4.0 eq) and sodium triacetoxyborohydride (3.0 eq), and the reaction mixture is stirred. Upon completion of the reaction, the reaction was quenched NH₄Cl (10% in water) the solution is filtered and washed with water. To the solution was added MeTHF, and the resulting mixture was basified with NaOH (15%) to pH 8-10. The layers were separated, and the aqueous layer was further extracted with MeTHF. The MeTHF layers were combined and washed with water to remove residual NMP. The organic layer was concentrated and seeded with compound R. To this mixture was added methylcyclohexane, and the mixture was cooled. The slurry was filtered and washed to obtain compound R in >80% yield.

Deprotection of Compound R: Formation of Compound S. To Compound R was added 5% H₂SO₄, and mixture is heated and stirred. The reaction was quenched by the addition of 16% NaOH solution to basify to pH 6-8. The solution was washed with DCM and 20% K₂CO₃ solution was added to adjust the pH to 9-11. The layers were separated, and the aqueous layer was further extracted with DCM. The organic layers were combined, and the solution was solvent swapped from DCM to THF. As a solution in THF, compound PB was isolated by heating and then cooling to RT followed by the addition of MBTE to allow for further precipitation. The slurry was stirred overnight. The slurry was filtered and washed to obtain compound S in >70% yield.

SNAR: Synthesis of Compound U. To a solution of compound S, compound T, and DMAc was added Na₂CO₃. The mixture was then stirred and heated overnight. The reaction was cooled and filtered to remove inorganics. The reaction was then quenched with NH₄Cl, and DCM was added. The layers were separated, and the aqueous layer was washed with DCM once again, and then organic layers were combined. The combined organic layers were washed with NH₄HCO₃. The layers were separated, and the NH₄HCO₃ process was repeated. The layers were separated, and the organic layer was concentrated. MeCN was added, and the mixture was concentrated to remove DCM. The mixture was then seeded with compound U, cooled, and stirred overnight. The slurry was then filtered and dried to provide compound U in >60% yield.

Synthesis of Compound S-D1. To compound U and compound V was added a THF/MeOH mixture. This solution was cooled and stirred, and AcOH was added. NaCNBH₃ was then added, while the mixture continued cooling. The reaction mixture was then allowed to stir overnight at RT until reaction completion. The reaction was quenched by the addition of a mixture of DCM/DMSO and Na₂CO₃. The layers were separated, and the organic layer was washed with Na₂CO₃ and then brine. The organic layer were separated and concentrated. To the concentrated organic layer was added MeCN, and the mixture was filtered. The filtrate was then further concentrated, and DMF was added. To the DMF solution was added water at RT, and the mixture was stirred for 10 h. The slurry was filtered, and drying the filter cake afforded compound S-D1 in ˜60% yield.

Recrystallization of Compound S-D1. Compound S-D1 was dissolved in DMSO (alternatively, NMP or DMF may be used), and a mixture of THF (or acetone)/water was then added. The mixture was seeded, and then further THF (or acetone)/water was added. The slurry was then filtered and dried to give compound S-D1 in 75-100% yield.

### Example 15—Forced Degradation Study of Compound D1

### Solution Preparation

Diluent 1: H₂O
Diluent 2: dimethylsulfoxide (DMSO)
Diluent 3: [0.05% formic acid (FA) in acetonitrile (MeCN)]: H₂O=1:1 (v/v)
2 N HCl was prepared by weighing 19.7076 g concentrated aq. HCl (37%) into a 100 ml volumetric flask containing approximately 20 mL diluent 1, diluting to volume with diluent 1, and mixing well.
0.002 N HCl was prepared as follows. 1 mL of 2 N HCl into was transferred into a 100 mL volumetric flask containing approximately 20 mL diluent 1, diluted to volume with diluent 1, and mixed well. Then, 1 mL of this solution was pipetted into a 10 mL volumetric flask, diluted to volume with diluent 1, and mixed well.
Diluent 4: 0.001 N HCl was prepared as follows. 1 mL of the 2 N HCl was transferred into a 100 mL volumetric flask containing approximately 20 mL diluent 1, diluted to volume with diluent 1, and mixed well. Then, 5 mL of this solution were pipetted into a 100 mL volumetric flask, made up to volume with diluent 1 and mixed well.
2 N NaOH was prepared as follows. 8.0248 g of NaOH were weighed into a 100 mL volumetric flask, diluted to volume with diluent 1, and mixed well.
0.1 N NaOH was prepared as follows. 5 mL of the 2 N NaOH were transferred into a 100 mL volumetric flask containing approximately 5 mL diluent 1, diluted to volume with diluent 1, and mixed well.
0.002 N NaOH was prepared as follows. 1 mL of the 2 N NaOH was transferred into a 100 mL volumetric flask containing approximately 20 mL diluent 1, diluted to volume with diluent 1, and mixed well. Then, 10 mL of this solution were pipetted into a 100 mL volumetric flask, diluted to volume with diluent 1, and mixed well.
6% Hydrogen peroxide was prepared as follows. 20 mL of concentrated H₂O₂ (30%) were transferred to a 100 mL volumetric flask, diluted to volume with diluent 1, and mixed well.
92% relative humidity control was achieved using a dryer containing a saturated potassium nitrate solution.
1 N Citric acid was prepared as follows. 19.2032 g of citric acid were weighed into a 100 mL volumetric flask, diluted to volume with diluent 1, and mixed well.
pH=4.0 50 mM Sodium citrate buffer was prepared as follows. 1.4814 g of sodium citrate were weighed into a 100 mL volumetric flask, diluted up to volume with diluent 1, and mixed well. pH was adjusted to 4.0 by 1 N citric acid.
pH=5.0 50 mM Sodium citrate buffer was prepared as follows. 1.4815 g of sodium citrate were weighed into a 100 mL volumetric flask, diluted to volume with diluent 1, and mixed well. pH was adjusted to 5.0 by 1 N citric acid.
pH=7.0 Phosphate buffer was prepared as follows. 0.6963 g of KH₂PO₄ were weighed into a 100 mL volumetric flask, 29.1 mL of the 0.1 N NaOH were transferred into the same volumetric flask, diluted to volume with diluent 1, and mixed well.

### Sample Preparation

Sample stock solution was prepared by weighing 60.74 mg of compound D1 to a 100 mL volumetric flask, dissolving by ultrasonic in 50 mL of diluent 2, diluting to volume with diluent 2, and mixing well.

Unstressed sample solution was prepared by weighing 21.32 mg of compound D1 into a 100 ml volumetric flask, dissolving by ultrasonic in 50 mL of diluent 3, diluting to volume with diluent 3, and mixing well.

Sensitivity solution (LOQ) was prepared as follows. 1.0 mL of unstressed sample solution was pipetted into a 100 mL volumetric flask, diluted to volume with diluent 3, and mixed well. Then, 5 mL of this sample solution were pipetted into a 100 mL volumetric flask, diluted to volume with diluent 3, and mixed well.

Sample solution for pH=4.0 solution stability was prepared as follows. 20.96 mg of compound D1 were weighed into a 100 mL volumetric flask, dissolved by ultrasonic in 50 ml of pH=4.0 50 mM sodium citrate buffer, diluted to volume with pH=4.0 50 mM sodium citrate buffer, and mixed well.

Sample solution for pH=5.0 solution stability was prepared as follows. 20.75 mg of compound D1 were weighed into a 100 mL volumetric flask, dissolved by ultrasonic in 50 ml of pH=5.0 50 mM sodium citrate buffer, diluted to volume with pH=5.0 50 mM sodium citrate buffer, and mixed well.

Sample solution for pH=7.0 solution stability was prepared as follows. 19.91 mg of compound D1 and 684.71 mg KH₂PO₄ were weighed into a 100 mL volumetric flask, dissolved by ultrasonic in 50 mL of diluent 1, transferred 29.1 mL of 0.1 N NaOH into the same volumetric flask, diluted to volume with diluent 1, and mixed well.

### Forced Degradation

### Stress Conditions

TABLE 8
Stress Conditions and Sampling time points

| Degradation type | Stress Solvent/Conditions | Time Point |
|---|---|---|
| Acid | 1N HCl at RT | 0 h, 1 d, 2 d, 3 d |
| Hydrolysis | | |
| Basic | 0.001N NaOH at RT | 0 h, 1 h |
| Hydrolysis | | |
| Oxidation | 3% Hydrogen Peroxide at RT | 0 h, 8 h |
| Photolysis | 1.10 W/m²/25° C. for solid | 13 h, 26 h, 39 h |
| | (1.10 W/m²/25° C. with | |
| | 13 hours equal to visible | |
| | 1.2M lux hrs and UV | |
| | 647 Wh/m²) | |
| Thermal | 80° C. for solid | 1 d, 3 d, 7 d |
| | 80° C. for solution | 1 d, 7 d |
| Humidity | 92% RH | 1 d, 3 d, 7 d |
| Solution | pH = 4.0, 50 mM Sodium | 0 h, 1 d, 3 d, 7 d |
| Stability | citrate buffer at RT | |
| | pH = 5.0, 50 mM Sodium | 0 h, 1 d, 3 d, 7 d |
| | citrate buffer at RT | |
| | pH = 7.0 Phosphate | 0 h, 1 d, 3 d, 7 d |
| | buffer at RT | |

Notes: 1. The target endpoint of a stress study was to form approximately 5-15% of total degradation product. 2. Based on actual degradation of the sample, the stress conditions including concentration of sample and reagent, and temperature, humidity, light may be adjusted. 3. After stressing samples in acid and base, neutralize them before placing into freezer. 4. All degradable samples before analysis must be placed into the 2° C. to 8° C. condition.

Acid degradation (1 N HCl at RT). 3 mL of the sample stock solution (see above) were transferred into an 8 mL vial, 3 mL of 2 N HCl solution were added, and the resulting mixture was mixed well. Samples were prepared in quadruplicate and kept at RT. At the sampling point, 2 mL of the sample were transferred into an 8 mL vial and neutralized with 1 mL of 2 N NaOH.

Blank: a blank has been prepared following the same procedure as described above, only without the inclusion of compound D1.

Basic degradation (0.001 N NaOH at RT). 3 mL of the sample stock solution (see above) were transferred into an 8 mL vial, 3 mL of 0.002 N NaOH solution were added, and the resulting mixture was mixed well. Samples were prepared in triplicate and kept at RT. At the sampling point, 2 mL of the solution were transferred into an 8 mL vial and neutralized with 1 mL of 0.002 N HCl.

Blank: a blank has been prepared following the same procedure as described above, only without the inclusion of compound D1.

Oxidation degradation (3% H₂O₂ at RT). 3 mL of the sample stock solution (see above) were transferred into an 8 mL vial, 3 mL of 6% H₂O₂ solution were added, and the resulting mixture was mixed well. Samples were prepared in quadruplicate and kept at RT. At the sampling time point, 2 mL of the solution were transferred into an 8 mL vial, neutralized with 1 mL of diluent 3, and mixed well.

Blank: a blank has been prepared following the same procedure as described above, only without the inclusion of compound D1.

Photolysis degradation (solid). About 60 mg of compound D1 were placed onto a watch glass. Samples were prepared in triplicate and placed in a photo chamber (see Table 8). At the sampling time point, the sample was placed in a vial for analysis. About 20.0±2.0 mg of the sample into a 100 mL volumetric flask, dissolved by ultrasonic in 50 mL of diluent 3, diluted to volume with diluent 3, and mixed well.

TABLE 9
Photolysis degradation
of samples' mass weight

| Sampling Name* | Weight (mg) |
|---|---|
| PSD-13 h | 20.44 |
| PSD-26 h | 19.93 |
| PSD-39 h | 19.43 |

*PSD-13 h, PSD-26 h and PSD-39 h in category mean the compound is degradation product under photolysis solid stress condition in 13 h, 26 h, and 39 h, respectively.

Dark control sample: one sample was prepared as described above for this test, but the watch glass was covered with aluminum foil, and processed as described above.

### Thermal Stress Degradation (80° C. for Solid and Solution).

Solid: about 60 mg of compound D1 were weighted into an 8 mL vial. Samples were prepared in triplicate and placed in an oven at 80° C. At the sampling time point, about 20.0±2.0 mg of the sample were weighed into a 100 mL volumetric flask, dissolved by ultrasonic in 50 ml of diluent 3, diluted to volume with diluent 3, and mixed well.

TABLE 10
Thermal degradation of
samples' mass weight

| Sample Name* | Weight (mg) |
|---|---|
| T-1 d | 19.82 |
| T-3 d | 22.07 |
| T-7 d | 21.68 |

*T-1 d, T-3 d and T-7 d in category mean the compound is degradation product under thermal solid stress condition in 1 d, 3 d and 7 d, respectively.

Solution: 3 mL of the sample stock solution (see above) were transferred into an 8 mL vial. Samples were prepared in triplicate and placed them in an oven at 80° C. At the sampling time point, 1 mL of the solution was transferred into an 8 mL vial, neutralized with 2 mL of diluent 3, and mixed well.

Blank: a blank has been prepared following the same procedure as described above, only without the inclusion of compound D1.

Humidity degradation (92% RH for solid). About 60 mg of compound D1 were transferred into a 20 mL vial (without cap). Samples were prepared in triplicate and placed in a desiccator at 92% RH. At the sampling time point, about 20.0±2.0 mg of the sample were transferred into a 100 mL volumetric flask, dissolved by ultrasonic in 50 mL of diluent 3, diluted to volume with diluent 3, and mixed well.

TABLE 11
Humidity degradation of
samples' mass weight

| Sample Name* | Weight (mg) |
|---|---|
| H-1 d | 21.02 |
| H-3 d | 21.41 |
| H-7 d | 20.11 |

*H-1 d, H-3 d and H-7 d in category mean the compound is degradation product under humidity solid stress condition in 1 d, 3 d and 7 d, respectively.

Solution Stability. Samples were prepared tested under the solution stability conditions noted in Table 8. The sampling time points were as noted in Table 8.

Blank: a blank has been prepared following the same procedure as described above, only without the inclusion of compound D1.

### Sample Analysis

TABLE 12
HPLC Method 1

| Mobile Phase A | H₂O + 0.1% TFA | | |
|---|---|---|---|
| Mobile Phase B | MeCN + 0.1% TFA | | |
| Column | Poroshell 120 SB-AQ | | |
| | (3.0*150 mm, 2.7 μm) | | |
| Needle Wash | MeCN: H₂O = 1:1 | | |
| Injection volume | 3 | μL | |
| Column Temperature | 40° | C. | |
| Flow Rate | 0.8 | mL/min | |
| Detection | 210 | nm | |
| Gradient | Time (min) | % A | % B |
| | 0.0 | 198 | 2 |
| | 7.0 | 70 | 30 |
| | 12.0 | 50 | 40 |
| | 15.0 | 5 | 195 |
| | 17.0 | 5 | 195 |

| | |
|---|---|
| Post time | 5 min |
| Run time | 17 min |

TABLE 13
Summary

| | | | | | | | | | % |
|---|---|---|---|---|---|---|---|---|---|
| Degradation | Stress Solvent/ | | Sample | Sample | | | | % | Mass |
| Type | Conditions | | Name | Number | Time | | Area % | Degradation | Balance |
| Unstressed | N/A | | N/A | 0 | N/A | | 94.172 | N/A | N/A |
| Acid | 1N HCl RT | | A 0 | 1 | 0 | h | 93.734 | 0.438 | 108.2 |
| Hydrolysis | | | A 1 | 2 | 1 | d | 89.391 | 4.781 | 106.5 |
| | | | A 2 | 3 | 2 | d | 85.180 | 8.992 | 107.6 |
| | | | A 3 | 4 | 3 | d | 82.047 | 12.125 | 106.9 |
| Base | 1N NaOH RT | | B 0 | 5 | 0 | h | 68.962 | 25.210 | 104.0 |
| Hydrolysis | | | B 1 | 6 | 1 | h | 17.331 | 76.841 | 98.0 |
| Oxidation | 3% H₂O₂ RT | | O 0 | 7 | 0 | h | 77.988 | 16.184 | 106.8 |
| | | | O 8 | 8 | 8 | h | 33.755 | 60.417 | 105.2 |
| Photolysis | 1.10 W/m²/25° C. for | | P 1 | 9 | 13 | h | 91.084 | 3.088 | 101.0 |
| | solid with | | P 2 | 10 | 26 | h | 90.916 | 3.256 | 92.1 |
| | 13 hours equal to | | P 3 | 11 | 39 | h | 90.736 | 3.436 | 105.7 |
| | visible 1.2M lux hrs | | | | | | | | |
| | and UV 647 Wh/m² | | | | | | | | |
| Thermal | 80° C. | Solid | TSD 1 | 12 | 1 | d | 93.448 | 0.724 | 105.7 |
| | | | TSD 3 | 13 | 3 | d | 93.194 | 0.978 | 97.6 |
| | | | TSD 7 | 14 | 7 | d | 82.192 | 11.980 | 92.7 |
| | 80° C. | Solution | TSN 1 | 15 | 1 | d | 94.053 | 0.119 | 100.5 |
| | | | TSN 7 | 16 | 7 | d | 31.937 | 62.235 | 104.2 |

| Humidity | 92% RH | H 1 | 17 | 1 | d | 92.927 | 1.245 | 98.8 |
|---|---|---|---|---|---|---|---|---|
| | | H 3 | 18 | 3 | d | 92.081 | 2.091 | 99.5 |
| | | H 7 | 19 | 7 | d | 90.890 | 3.282 | 99.8 |
| Solution | pH = 4.0, 50 mM | pH 4 0 | 20 | 0 | h | 93.516 | 0.656 | 90.1 |
| Stability | Sodium citrate | pH 4 1 | 21 | 1 | d | 93.331 | 0.841 | 93.7 |
| | buffer at RT | pH 4 3 | 22 | 3 | d | 92.873 | 1.299 | 93.9 |
| | | pH 4 7 | 23 | 7 | d | 92.130 | 2.042 | 93.7 |
| | pH = 5.0 50 mM | pH 5 0 | 24 | 0 | h | 92.498 | 1.674 | 95.3 |
| | Sodium citrate | pH 5 1 | 25 | 1 | d | 92.250 | 1.922 | 103.1 |
| | buffer at RT | pH 5 3 | 26 | 3 | d | 92.039 | 2.133 | 100.0 |
| | | pH 5 7 | 27 | 7 | d | 89.514 | 4.658 | 100.8 |
| | pH = 7.0 Phosphate | pH 7 0 | 28 | 0 | h | 92.638 | 1.534 | 97.1 |
| | buffer at RT | pH 7 1 | 29 | 1 | d | 85.252 | 8.920 | 102.6 |

| | | | | | | |
|---|---|---|---|---|---|---|
| pH 7 2 | 30 | 2 | d | 79.002 | 15.170 | 100.6 |

TABLE 14
Summary result of mass balance, resolution and purity factor

| | | Mass Balance | | | *Resolution | | | Purity Factor | | |
|---|---|---|---|---|---|---|---|---|---|---|
| Sample | | | | Pass/ | | | Pass/ | | | Pass/ |
| Number | Purity % | Result | Criteria | Fail | Result | Criteria | Fail | Result | Criteria | Fail |
| 4 | 82.047 | 106.9 | 90%-110% | Pass | 2.0 | ≥1.2 | Pass | 999.943 | >990 | Pass |
| 6 | 17.331 | 98.0 | | | 1.8 | | | 996.409 | | |
| 8 | 33.755 | 105.2 | | | 1.0 | | Fail | 998.510 | | |
| 11 | 90.736 | 105.7 | | | 1.8 | | Pass | 999.780 | | |
| 14 | 82.192 | 92.7 | | | 1.8 | | | 999.941 | | |
| 16 | 31.937 | 104.2 | | | 1.8 | | | 999.708 | | |
| 19 | 90.890 | 99.8 | | | 1.9 | | | 999.438 | | |
| 23 | 92.130 | 93.7 | | | 2.0 | | | 999.830 | | |
| 27 | 89.514 | 100.8 | | | 2.0 | | | 999.639 | | |
| 30 | 79.002 | 100.6 | | | 2.0 | | | 998.160 | | |

*Resolution was the adjacent impurity with the main peak.
According to the result above, samples 6 and 8 were used to method optimized.

### Example 16—Forced Degradation Study of Compound S-D1

### Solutions Preparation

Mobile phase: methanol (MeOH):acetonitrile (MeCN)=3:7 (v/v)+25 mM formic acid (FA)+25 mM NH₃. Prepared by transferring 300 mL MeOH, 700 mL MeCN, 970 μL FA, and 12.5 mL Ammonia in to 1 L bottle, mixed well.

Needle wash solution: MeOH
Diluent 1: H₂O
Diluent 2: 0.05% FA in MeCN:THF=1:1 (v/V)
2 N HCl was prepared by weighing 19709.02 mg concentrated aq. HCl (37%) into a 100 mL volumetric flask containing approximately 20 mL diluent 1, diluting to volume with diluent 1, and mixing well.
0.3 N HCl was prepared by transferring 15 mL of the 2 N HCl (see above) into a 100 ml volumetric flask containing approximately 20 mL diluent 1, diluting to volume with diluent 1, and mixing well.
2 N NaOH was prepared by weighing 7996.77 mg NaOH into a 100 mL volumetric flask, made up to volume with diluent 1 and mixed well.
0.3 N NaOH was prepared by transferring 15 mL of the 2 N NaOH (see above) into a 100 ml volumetric flask containing approximately 5 mL diluent 1, diluting to volume with diluent 1, and mixing well.
0.1 N NaOH was prepared by transferring 5 mL of the 2 N NaOH (see above) into a 100 mL volumetric flask containing approximately 5 mL diluent 1, diluting to volume with diluent 1, and mixing well.
1 N Citric acid was prepared by weighing 19193.47 mg citric acid into a 100 mL volumetric flask, diluting to volume with diluent 1, and mixing well.
pH=4.0 50 mM Sodium citrate buffer was prepared by weighing 1474.25 mg sodium citrate into a 100 mL volumetric flask, diluting to volume with diluent 1, and mixing well. pH was adjusted pH to 4.0 by 1 N citric acid.
pH=5.0 50 mM Sodium citrate buffer was prepared by weighing 1474.11 mg sodium citrate into a 100 mL volumetric flask, diluting to volume with diluent 1, and mixing well. pH was adjusted to 5.0 by 1 N citric acid.

Sample solution stock was prepared by weighing 501.53 mg of compound S-D1 to a 100 ml volumetric flask, dissolving by ultrasonic in 50 mL of diluent 2, diluting to volume with diluent 2, and mixing well.

Unstressed sample solution was prepared by weighing 50.83 mg of compound S-D1 into a 100 mL volumetric flask, dissolving by ultrasonic in 50 mL of diluent 2, diluting to volume with diluent 2, and mixing well.

Sensitivity solution (LOQ) was prepared as follows. 1.0 mL of unstressed sample solution were pipetted into a 100 mL volumetric flask, diluted to volume with diluent 2, and mixed well. Then 2 mL of this sample solution were pipetted into a 10 mL volumetric flask, diluted to volume with diluent 2, and mixed well.

Sample solution for pH=4.0 solution stability was prepared as follows. 50.79 mg of compound S-D1 were weighed into a 100 mL volumetric flask, dissolved by ultrasonic in 50 mL of pH=4.0 50 mM sodium citrate buffer (see above), diluted to volume with pH=4.0 50 mM sodium citrate buffer, and mixed well.

Sample solution for pH=5.0 solution stability was prepared as follows. 50.66 mg of compound S-D1 were weighed into a 100 mL volumetric flask, dissolved by ultrasonic in 50 ml of pH=5.0 50 mM sodium citrate buffer (see above), diluted to volume with 50 ml of pH=5.0 50 mM sodium citrate buffer and mixed well.

Sample solution for pH=7.0 solution stability was prepared as follows. 50.78 mg of compound S-D1 and 680.42 mg KH₂PO₄ were weighed into a 100 mL volumetric flask, dissolved by ultrasonic in 50 mL of diluent 1, transferred 29.1 mL of the 0.1 N NaOH (see above), diluted to volume with diluent 1, and mixed well.

### Forced Degradation

TABLE 15
Stress Conditions and Sampling Time Points

| Degradation | Stress | Sampling |
|---|---|---|
| type | Solutions/Conditions | Time Point |
| Acid Hydrolysis | 1N HCl at RT | 0 h, 1 d, 2 d, 3 d |
| Basic Hydrolysis | 0.15N NaOH at RT | 0 h, 1 h |
| Thermal | 50° C. for solid | 1 d, 3 d, 7 d |
| Solution | pH = 4.0, 50 mM Sodium | 0 h, 1 d, 2 d, 3 d |
| Stability | citrate buffer at RT | |
| | pH = 5.0, 50 mM Sodium | 0 h, 1 d, 2 d, 3 d |
| | citrate buffer at RT | |
| | pH = 7.0 Phosphate | 0 h, 1 d, 2 d, 3 d |
| | buffer at RT | |

Note:
After stressing samples in acid or base, the samples were neutralized before placing into freezer.

Acid degradation (1 N HCl at RT). 3 mL of the sample stock solution (see above) into an 8 mL vial, added 3 mL of 2 N HCl solution, and mixed well. Samples were prepared in quadruplicate and kept at room temperature (RT). At the sampling time point, 1 mL of the sample was transferred into a 5 mL volumetric flask, neutralized with 0.5 mL of 2 N NaOH, diluted to volume with diluent 2, and mixed well.

Blank: a blank has been prepared following the same procedure as described above, only without the inclusion of compound S-D1.

Basic degradation (0.15 N NaOH at RT). 3 mL of the sample stock solution (see above) into an 8 mL vial, added 3 mL of 0.3 N NaOH solution, and mixed well. Samples were prepared in quadruplicate and kept at room temperature (RT). At the sampling time point, 1 mL of the sample was transferred into a 5 mL volumetric flask, neutralized with 0.5 mL of 2 N NaOH, diluted to volume with diluent 2, and mixed well.

Blank: a blank has been prepared following the same procedure as described above, only without the inclusion of compound S-D1.

Thermal stress degradation (50° C. for solid). Approximately 150 mg of compound S-D1 were weighed into an 8 mL vial. Multiple samples were prepared and placed in an oven at 50° C. At the sampling time points, each of the 50.57 mg, 50.84 mg, and 50.84 mg samples were transferred into their respective 100 mL volumetric flasks, dissolved by ultrasonic in 50 mL of diluent 2, diluted to volume with diluent 2, and mixed well.

Solution Stability. The study was performed according to the conditions outlined in Table 15 using sample solution for pH=4.0 solution stability, sample solution for pH=5.0 solution stability, and sample solution for pH=7.0 solution stability (see above).

Blank: a blank has been prepared following the same procedure as described above, only without the inclusion of sample.

### Sample Analysis

TABLE 16
HPLC Method

| Mobile Phase | MeOH:MeCN = 3:7 (v/v) + | |
|---|---|---|
| | 25 mM FA + 25 mM NH₃ | |
| Column | Cellulose SB, 100*4.6 mm, 3.0 μm | |
| Needle Wash | MeOH | |
| Injection volume | 1 | μL |
| Column Temperature | 30° | C. |
| Flow Rate | 0.8 | mL/min |
| Diluent | 0.05% FA in | |
| | MeCN:THF = 1:1 (v/V) | |
| Autosampler | RT | |
| Temperature | | |
| Detection | 280 nm | |
| Isocratic | Time (minute) | Mobile Phase (%) |
| | 0.0 | 100 |
| | 8.0 | 100 |

| | |
|---|---|
| Data Record Time | 8 minutes |

TABLE 17
Summary

| Degradation | Stress | | Sample | Area % | Area % | % |
|---|---|---|---|---|---|---|
| Type | Solvent/Conditions | Time | Number | (R-D1) | (S-D1) | Degradation |
| Unstressed | N/A | N/A | 0 | 0.823 | 99.177 | N/A |
| Acid | 1N HCl RT | 0 h | 1 | 0.825 | 99.176 | 0.002 |
| Hydrolysis | | 1 d | 2 | 0.828 | 99.172 | 0.005 |
| | | 2 d | 3 | 0.834 | 99.166 | 0.011 |
| | | 3 d | 4 | 0.839 | 99.161 | 0.016 |
| Base | 0.15N NaOH RT | 0 h | 5 | 19.083 | 80.917 | 18.260 |
| Hydrolysis | | 1 h | 6 | 53.033 | 46.967 | 52.210 |
| Thermal | 50° C. | 1 d | 7 | 1.001 | 98.999 | 0.178 |
| | | 3 d | 8 | 1.154 | 98.847 | 0.331 |
| | | 7 d | 9 | 1.203 | 98.797 | 0.380 |
| Solution | pH = 4.0, | 0 h | 10 | 0.921 | 99.079 | 0.098 |
| Stability | 50 mM Sodium | 1 d | 11 | 0.962 | 99.038 | 0.139 |
| | citrate buffer at RT | 2 d | 12 | 1.124 | 98.876 | 0.301 |
| | | 3 d | 13 | 1.305 | 98.695 | 0.482 |
| | pH = 5.0 | 0 h | 14 | 1.471 | 98.529 | 0.648 |
| | 50 mM Sodium | 1 d | 15 | 1.861 | 98.139 | 1.038 |
| | citrate buffer at RT | 2 d | 16 | 2.230 | 97.770 | 1.407 |
| | | 3 d | 17 | 2.825 | 97.175 | 2.002 |
| | pH = 7.0 | 0 h | 18 | 11.555 | 88.445 | 10.732 |
| | Phosphate buffer at | 1 d | 19 | 19.745 | 80.255 | 18.922 |
| | RT | 2 d | 20 | 28.374 | 71.626 | 27.551 |
| | | 3 d | 21 | 35.926 | 64.074 | 35.103 |

TABLE 18
Summary result of resolution

| | Resolution between | | | Resolution between | | | | | |
|---|---|---|---|---|---|---|---|---|---|
| | compound R-D1 peak | | | compound S-D1 peak | | | | | |
| | and its adjacent peak | | | and its adjacent peak | | | Purity Factor | | |
| Sample | | | Pass/ | | | Pass/ | | | Pass/ |
| Number | Result | Criteria | Fail | Result | Criteria | Fail | Result | Criteria | Fail |
| 4 | 1.3 | ≥1.2 | Pass | 2.0 | ≥1.2 | Pass | 999.917 | >990 | Pass |
| 6 | 1.8 | | | 4.4 | | | 996.140 | | |
| 9 | 1.8 | | | 2.3 | | | 999.152 | | |
| 13 | 1.7 | | | 3.7 | | | 999.947 | | |
| 17 | 1.9 | | | 4.1 | | | 999.616 | | |
| 21 | 2.1 | | | 3.9 | | | 999.728 | | |

### Example 17—Stability of Compound S-D1 in Blood and Blood Plasma

Compound S-D1 (1 μM) was incubated at 37° C. in triplicates with plasma and blood of human, monkey, and rat, and samples were taken from each incubation at 0, 30, 60, 120 and 240 min. Propantheline (5 UM, human and monkey) or mevinolin (5 UM, rat) were used as a positive control for plasma and blood stability. Samples were analyzed by Ultra Performance Liquid Chromatography with Tandem Mass Spectrometric Detection (UPLC/MS-MS). Throughout the study, control compounds performed as expected.

LC conditions were as follows:

| Instrument: | Shimadzu 30 AD | | | |
|---|---|---|---|---|
| Column: | Waters XSelect ®HSS | | | |
| | T3 2.5 μm (2.1 × 50 mm) | | | |
| Column | 40° C. | | | |
| temperature: | | | | |
| Mobile phase: | A: 0.1% formic acid in water | | | |
| | B: 0.1% formic acid in acetonitrile | | | |
| Injection volume: | 1 μL | | | |
| | Time | A | B | Elution rate |
| | (min) | (%) | (%) | (mL/min) |
| | 0 | 95 | 5 | 0.7 |
| | 0.2 | 95 | 5 | 0.7 |
| | 0.7 | 2 | 98 | 0.7 |
| | 1.2 | 2 | 98 | 0.7 |
| | 1.25 | 95 | 5 | 0.7 |
| | 1.5 | 95 | 5 | 0.7 |

Mass conditions were as follows:

| | |
|---|---|
| Instrument: | Triple Quad 5500 (AB sciex, USA) |
| Ion source: | Turbo spray |
| Ionization model: | ESI |
| Scan type: | MRM |
| Ionization mode: | Positive |

Mass spectrometer conditions:

| Test Article | Q1 (m/z) | Q3 (m/z) | DP (v) | EP(v) | CE(v) | CXP(v) |
|---|---|---|---|---|---|---|
| Alprazolam | 309.1 | 281.1 | 80 | 10 | 36 | 10 |
| Mevinolin | 405.3 | 199 | 85 | 15 | 15 | 10 |
| Propantheline | 369.2 | 99.7 | 80 | 10 | 50 | 10 |

Plasma samples were prepared as follows. Plasma was acquired from suppliers and stored at −80° C. prior to use. A water bath was set to 37° C. Frozen plasma (stored at −80° C.) was thawed immediately prior to use in the 37° C. water bath. The plasma was centrifuged at 2,000 g for 5 minutes to remove clots and collect supernatant into a fresh tube. pH of the plasma was then checked. The present study only utilized plasma that was thawed once and was within the range of pH 7.2 to 8.0.

Ratios of compound R-D1 over compound S-D1 were calculated to assess the conversion from compound S-D1 to compound R-D1 in human, monkey, and rat plasma or blood incubated with compound S-D1 up to 4 h (Table 19). Under the experimental condition, ratios of compound R-D1 over compound S-D1 in plasma or blood increased over time and were generally similar among the three species evaluated. Ratios of compound R-D1 over compound S-D1 were ˜0.01 at time 0 and reached ˜0.6-0.7 in plasma or ˜0.1-0.2 in blood after 4 h of incubations.

TABLE 19
Ratios of compound R-D1 over compound S-D1
in human, monkey, and rat plasma or blood

| Matrix | Time (min) | Human | Monkey | Rat |
|---|---|---|---|---|
| Plasma | 0 | 0.00749 ± 0.00524 | 0.0138 ± 0.00170 | 0.0130 ± 0.00603 |
| | 30 | 0.0288 ± 0.00791 | 0.0630 ± 0.000692 | 0.0572 ± 0.00774 |
| | 60 | 0.0712 ± 0.00964 | 0.123 ± 0.00256 | 0.124 ± 0.00512 |
| | 120 | 0.268 ± 0.0220 | 0.279 ± 0.000599 | 0.300 ± 0.0472 |
| | 240 | 0.611 ± 0.0473 | 0.570 ± 0.0140 | 0.713 ± 0.0539 |
| Blood | 0 | 0.0140 ± 0.000573 | 0.0138 ± 0.00122 | 0.0141 ± 0.000410 |
| | 30 | 0.0332 ± 0.00319 | 0.0292 ± 0.000973 | 0.0237 ± 0.00113 |
| | 60 | 0.0500 ± 0.00223 | 0.0466 ± 0.00747 | 0.0344 ± 0.00133 |
| | 120 | 0.0975 ± 0.00138 | 0.0900 ± 0.00142 | 0.0569 ± 0.00227 |
| | 240 | 0.180 ± 0.00438 | 0.152 ± 0.000572 | 0.101 ± 0.00255 |

Values are means ± SD of triplicate determinations.

Percent remaining of total (compound R-D1 and compound S-D1) was calculated to assess degradation in human, monkey, and rat plasma or blood incubated with compound S-D1 to 4 h (Table 20). While degradation was minimal in human and rat blood, loss of total in human and rat plasma increased over time. After 4 h of incubations, ˜100% remaining in human and rat blood, and ˜60-70% remaining in human and rat plasma were observed. Degradation in monkey plasma and blood appeared to be similar with ˜80% remaining observed at 4 h.

TABLE 20
Percent Remaining of compounds R-D1 and S-D1
in human, monkey, and rat plasma or blood

| | Time | % remaining of total | | |
|---|---|---|---|---|
| Matrix | (min) | Human | Monkey | Rat |
| Plasma | 0 | 100 | 100 | 100 |
| | 30 | 94.5 ± 7.02 | 88.9 ± 3.81 | 90.4 ± 4.01 |
| | 60 | 96.1 ± 0.757 | 96.3 ± 2.41 | 88.3 ± 7.05 |
| | 120 | 84.2 ± 3.80 | 94.3 ± 6.75 | 78.0 ± 1.51 |
| | 240 | 72.6 ± 4.21 | 83.4 ± 4.58 | 63.0 ± 6.48 |
| Blood | 0 | 100 | 100 | 100 |
| | 30 | 100 ± 4.91 | 96.4 ± 5.46 | 115 ± 4.38 |
| | 60 | 101 ± 2.59 | 98.1 ± 5.70 | 112 ± 5.97 |
| | 120 | 98.5 ± 6.24 | 94.2 ± 7.93 | 95.1 ± 11.7 |
| | 240 | 99.9 ± 7.21 | 80.0 ± 4.56 | 115 ± 5.86 |

Values are means ± SD of triplicate determinations

### OTHER EMBODIMENTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1. A method of determining the level of a chiral glutarimide comprising an epimerizable stereogenic center at the α-carbon of the glutarimide ring in a subject, the method comprising:
collecting a body fluid from the subject into a container comprising a solution comprising citric acid to form a composition; and
determining the level of the chiral glutarimide in the composition,
thereby determining the level of a chiral glutarimide in a subject.

2. A method of preparing a composition comprising a chiral glutarimide comprising an epimerizable stereogenic center at the α-carbon of the glutarimide ring and a body fluid, the method comprising collecting the body fluid from a subject into a container comprising a solution comprising citric acid, the body fluid comprising the chiral glutarimide.

3. A composition comprising a chiral glutarimide comprising an epimerizable stereogenic center at the α-carbon of the glutarimide ring, a body fluid, and citrate buffer, wherein the composition is enriched for one of the stereoisomers of the epimerizable stereogenic center.

4. The method or composition of any one of claims 1 to 3, wherein the body fluid is blood.

5. The method or composition of any one of claims 1 to 4, wherein the solution comprising citric acid is 0.5M to 5M.

6. The method or composition of any one of claims 1 to 5, wherein the solution comprising citric acid is 3M citric acid.

7. The method or composition of any one of claims 1 to 6, wherein the ratio of body fluid to the solution of citric acid in the composition is 99 to 1.

8. A method of determining the level of a chiral glutarimide comprising an epimerizable stereogenic center at the α-carbon of the glutarimide ring in a subject, the method comprising:
collecting a body fluid from the subject;
separating the body fluid into two or more components;
combining one of the components and citric acid to form a composition; and
determining the level of the chiral glutarimide in the composition,
thereby determining the level of a chiral glutarimide in a subject.

9. A method of preparing a composition comprising a chiral glutarimide comprising an epimerizable stereogenic center at the α-carbon of the glutarimide ring and a component of a body fluid, the method comprising combining citric acid and the component of the body fluid collected from a subject.

10. A composition comprising a chiral glutarimide comprising an epimerizable stereogenic center at the α-carbon of the glutarimide ring, a component of a body fluid, and citrate buffer, wherein the composition is enriched for one of the stereoisomers of the epimerizable stereogenic center.

11. The method or composition of any one of claims 8 to 10, wherein the component of a body fluid is blood serum or blood plasma.

12. The method or composition of claim 11, wherein the component of a body fluid is blood serum.

13. The method or composition of claim 11, wherein the component of a body fluid is blood plasma.

14. The method or composition of any one of claims 8 to 13, wherein the citric acid is in lyophilized form.

15. The method or composition of any one of claims 8 to 14, wherein the ratio of the component of body fluid to citric acid in the composition is 99 to 1.

16. The method of any one of claims 8, 9, or 11 to 15, wherein the body fluid is stored at 0° C. to 4° C. between the step of collecting the body fluid and the step of preparing the component of the body fluid.

17. The method or composition of any one of claims 1 to 16, wherein the chiral glutarimide comprising an epimerizable stereogenic center at the α-carbon of the glutarimide ring is a compound of formula l:
A-L-B Formula I,
wherein
L is a linker;
B is a degradation moiety having the structure: 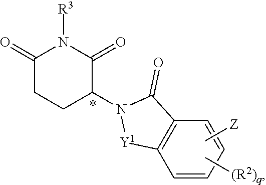
wherein
*designates the stereoenriched epimerizable stereogenic center at the α-carbon of the glutarimide ring;
Y¹ is 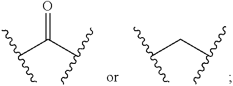
R³ is H, optionally substituted C₁-C₆ alkyl, or optionally substituted C₁-C₆ heteroalkyl;
q is 0, 1, 2, 3, or 4;
each R² is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; and
Z is a substituent; and
A is a protein binding moiety,
or a pharmaceutically acceptable salt thereof.

18. The method or composition of claim 17, wherein the protein binding moiety has the structure of Formula E-3, Formula E-4, Formula G-2, Formula G-3, or Formula E-5: 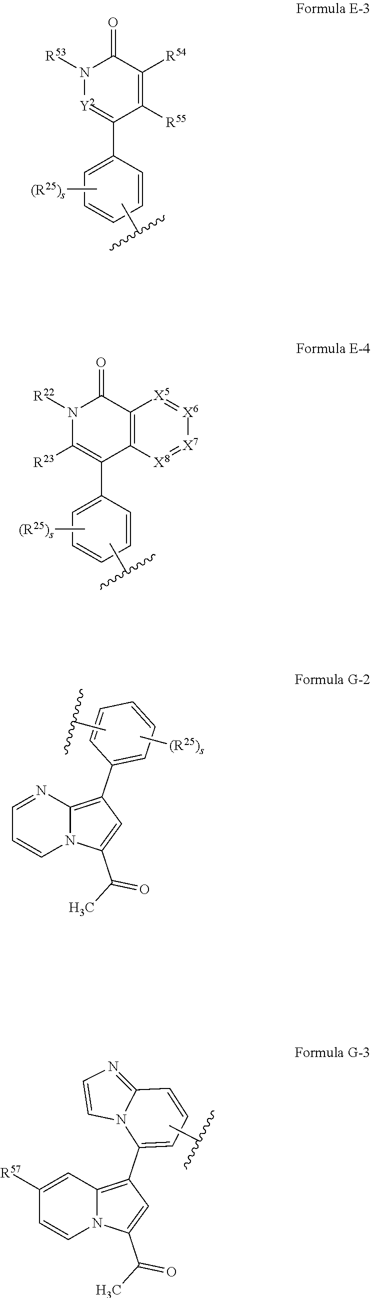 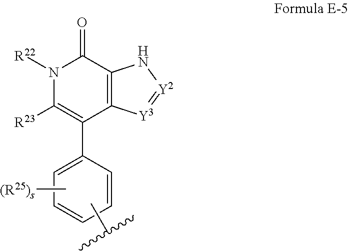
wherein
Y² is N or CR²³;
R²² is H, optionally substituted C₁-C₆ alkyl, or optionally substituted C₁-C₆ heteroalkyl;
R²³ is H, halogen, optionally substituted C₁-C₆ alkyl, or optionally substituted C₆-C₁₀ aryl;
s is 0, 1, 2, 3, or 4;
each R²⁵ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino;
R⁵³ is H, optionally substituted C₁-C₆ alkyl, optionally substituted C₁-C₆ heteroalkyl, or optionally substituted C₃-C₁₀ carbocyclyl;
R⁵⁴ is H or optionally substituted C₂-C₉ heteroaryl;
R⁵⁵ is H or N(R^{a})₂, wherein each R^{a} is independently H, optionally substituted C₁-C₆ alkyl, optionally substituted C₁-C₆ heteroalkyl, or optionally substituted C₃-C₁₀ carbocyclyl, or two geminal R^{a}, together with the nitrogen atom to which they are attached, combine to form optionally substituted C₂-C₉ heterocyclyl;
each of X⁵, X⁶, X⁷, and X⁸ is, independently, N or CR⁵⁶;
each R⁵⁶ is, independently, H or N(R^{a})₂, wherein R^{a} is H, optionally substituted C₁-C₆ alkyl, optionally substituted C₁-C₆ heteroalkyl, or optionally substituted C₃-C₁₀ carbocyclyl, or two geminal R^{a}, together with the nitrogen atom to which they are attached, combine to form optionally substituted C₂-C₉ heterocyclyl;
R⁵⁷ is optionally substituted C₂-C₁₀ heterocyclyl;
each of Y² and Y³ is, independently, N or CR⁵⁸; and
each R⁵⁸ is, independently, H or optionally substituted C₁-C₆ alkyl,
wherein if R⁵³ is H and R⁵⁴ is H, then R⁵⁵ is NR^{a}; if R⁵⁴ is H and R⁵⁵ is H, then R⁵³ is optionally substituted C₃-C₁₀ carbocyclyl; and if R⁵³ is H and R⁵⁵ is H, then R⁵⁴ is optionally substituted C₂-C₉ heteroaryl,
or a pharmaceutically acceptable salt thereof.

19. The method or composition of claim 17 or 18, wherein A has the structure of Formula E-3.

20. The method or composition of claim 17 or 18, wherein A has the structure of Formula E-4.

21. The method or composition of claim 17 or 18, wherein A has the structure of Formula G-2.

22. The method or composition of claim 17 or 18, wherein A has the structure of Formula G-3.

23. The method or composition of claim 17 or 18, wherein A has the structure of Formula E-5.

24. The method or composition of any one of claims 17 to 23, wherein s is 0, 1, or 2.

25. The method or composition of any one of claims 17 to 24, wherein the degradation moiety has the structure of Formula A-1: 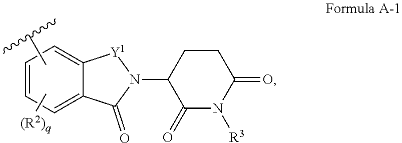
wherein
Y¹ is 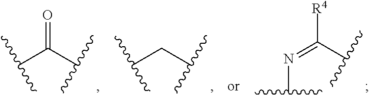
R³ and R⁴ are, independently, H, optionally substituted C₁-C₆ alkyl, or optionally substituted C₁-C₆ heteroalkyl;
q is 0, 1, 2, 3, or 4; and
each R² is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino,
or a pharmaceutically acceptable salt thereof.

26. The method or composition of claim 25, wherein R³ is H or optionally substituted C₁-C₆ alkyl.

27. The method or composition of claim 26, wherein R³ is H or CH₃.

28. The method or composition of claim 27, wherein R³ is H.

29. The method or composition of claim 27, wherein R³ is CH₃.

30. The method or composition of any one of claims 25 to 29, wherein Y¹ is

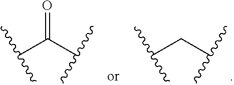

31. The method or composition of claim 30, wherein Y¹ is

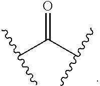 [SMILES: *C(*)=O]

32. The method or composition of any one of claims 25 to 31, wherein each R² is, independently, optionally substituted C₁-C₆ alkyl, optionally substituted C₁-C₆ heteroalkyl, hydroxyl, or optionally substituted amino.

33. The method or composition of any one of claims 25 to 34, wherein q is 0 or 1.

34. The method or composition of claim 33, wherein q is 0.

35. The method or composition of any one of claims 25 to 34, wherein the degradation moiety has the structure of Formula A-1a:

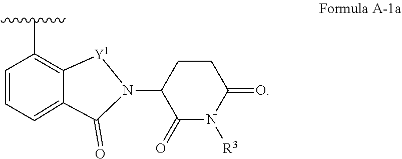

36. The method or composition of any one of claims 25 to 34, wherein the degradation moiety has the structure of Formula A-1b:

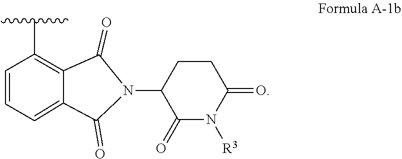

37. The method or composition of any one of claims 25 to 34, wherein the degradation moiety has the structure of Formula A-1c:

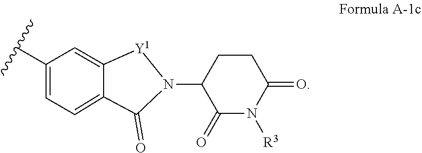

38. The method or composition of any one of claims 25 to 34, wherein the degradation moiety has the structure of Formula A-1d:

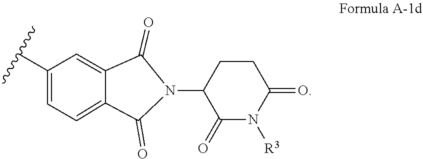

39. The method or composition of any one of claims 25 to 34, wherein the degradation moiety has the structure:

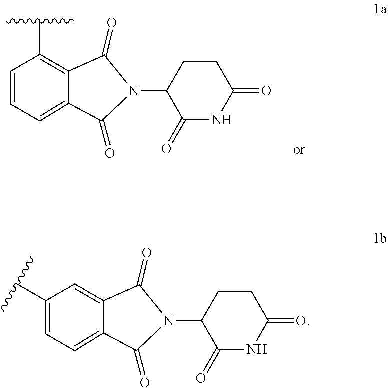 [SMILES: *c1cccc2c1C(=O)N(C1CCC(=O)NC1=O)C2=O ; *c1ccc2c(c1)C(=O)N(C1CCC(=O)NC1=O)C2=O]

40. The method or composition of any one of claims 17 to 39, wherein the linker has the structure of Formula II:
A¹-(B¹)_{f}-(C¹)_{g}-(B²)ₕ-(D)-(B³)ᵢ-(C²)ⱼ-(B⁴)ₖ-A² Formula II
wherein
A¹ is a bond between the linker and A;
A² is a bond between B and the linker;
each of B¹, B², B³, and B⁴ is, independently, optionally substituted C₁-C₂ alkyl, optionally substituted C₁-C₃ heteroalkyl, optionally substituted C₂₋₉ heterocyclyl, O, S, S(O)₂, or NR^{N};
R^{N} is H, optionally substituted C₁₋₄ alkyl, optionally substituted C₂₋₄ alkenyl, optionally substituted C₂₋₄ alkynyl, optionally substituted C₂₋₉ heterocyclyl, optionally substituted C₆₋₁₂ aryl, or optionally substituted C₁₋₇ heteroalkyl;
each of C₁ and C₂ is, independently, carbonyl, thiocarbonyl, sulphonyl, or phosphoryl;
f, g, h, I, j, and k are each, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₉ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}-(C¹)_{g}-(B²)ₕ-to-(B³)ᵢ-(C²)ⱼ-(B⁴)ₖ-A².

41. The method or composition of claim 40, wherein each of B¹, B², B³, and B⁴ is, independently, optionally substituted C₁-C₄ alkyl, optionally substituted C₁-C₄ heteroalkyl, or NR^{N}.

42. The method or composition of claim 40 or 41, wherein R^{N} is H or optionally substituted C₁₋₄ alkyl.

43. The method or composition of claim 42, wherein R^{N} is H or CH₃.

44. The method or composition of any one of claims 40 to 43, wherein each of B¹ and B⁴ is, independently,

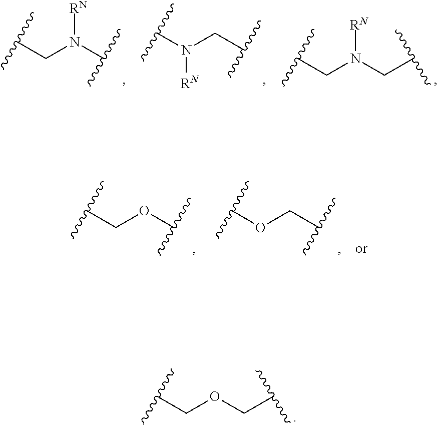

45. The method or composition of claim 44, wherein B¹ is

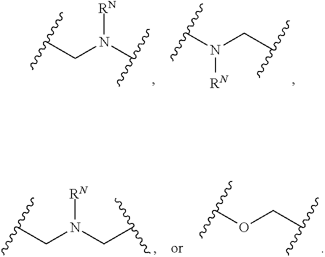

46. The method or composition of any one of claims 40 to 45, wherein each of C¹ and C² is, independently,

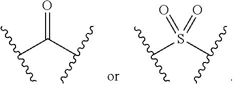

47. The method or composition of claim 46, wherein C¹ is

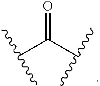 [SMILES: *C(*)=O]

48. The method or composition of any one of claims 1 to 47, wherein the chiral glutarimide has the structure:

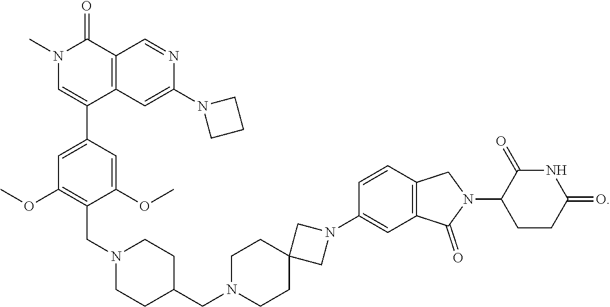 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N(C2CCC(=O)NC2=O)C4)C3)CC1]

49. The method or composition of any one of claims 1 to 48, wherein the chiral glutarimide has the structure:

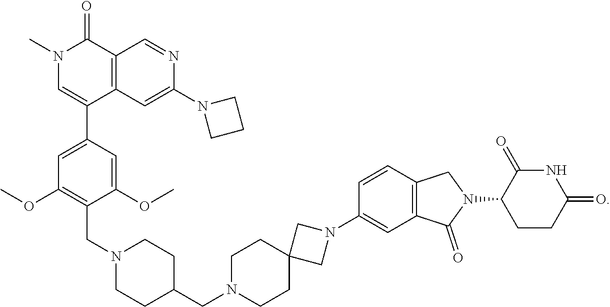 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N([C@H]2CCC(=O)NC2=O)C4)C3)CC1]

50. The method or composition of any one of claims 1 to 49, wherein the chiral glutarimide is enriched for one of the stereoisomers at the epimerizable stereogenic center.

51. A method of preparing a chiral glutarimide or a salt thereof comprising an epimerizable stereogenic center at the α-carbon of the glutarimide ring, the epimerizable stereogenic center being enriched for one of the stereoisomers, and the method comprising reacting a stereoenriched aminoglutarimide with a carboxybenzaldehyde,
wherein the chiral glutarimide is of the following structure: 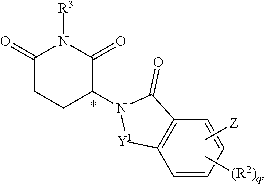
wherein
*designates the stereoenriched epimerizable stereogenic center at the α-carbon of the glutarimide ring;
Y¹ is 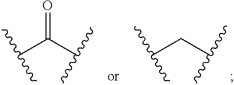
R³ is H, optionally substituted C₁-C₆ alkyl, or optionally substituted C₁-C₆ heteroalkyl;
q is 0, 1, 2, 3, or 4;
each R² is, independently, halogen, optionally substituted C₁-C₈ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino; and
Z is L-A;
wherein L is a linker; and
A is a protein binding moiety;
wherein the enantioenriched aminoglutarimide is of the following structure: 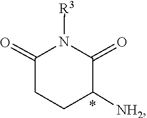
or a salt thereof,
wherein all variables are same as those in the chiral glutarimide;
and
wherein the carboxybenzaldehyde is of the following structure: 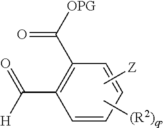
wherein
PG is an O-protecting group, and
all remaining variables are same as those in the chiral glutarimide.

52. The method of claim 51, wherein R³ is H or optionally substituted C₁-C₆ alkyl.

53. The method of claim 52, wherein R³ is H or CH₃.

54. The method of claim 53, wherein R³ is H.

55. The method of claim 53, wherein R³ is CH₃.

56. The method of any one of claims 51 to 55, wherein Y¹ is

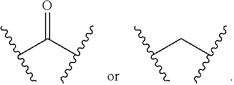

57. The method of claim 56, wherein Y¹ is

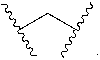 [SMILES: *C*]

58. The method of any one of claims 51 to 57, wherein each R² is, independently, optionally substituted C₁-C₆ alkyl, optionally substituted C₁-C₆ heteroalkyl, hydroxyl, or optionally substituted amino.

59. The method of any one of claims 51 to 58, wherein q is 0 or 1.

60. The method of claim 59, wherein q is 0.

61. The method of any one of claims 51 to 60, wherein the enantioenriched aminoglutarimide is reacted with the carboxybenzaldehyde under the reductive amination conditions.

62. The method of any one of claims 51 to 61, wherein
A has the structure of Formula E-3, Formula E-4, Formula G-2, Formula G-3, or Formula E-5: 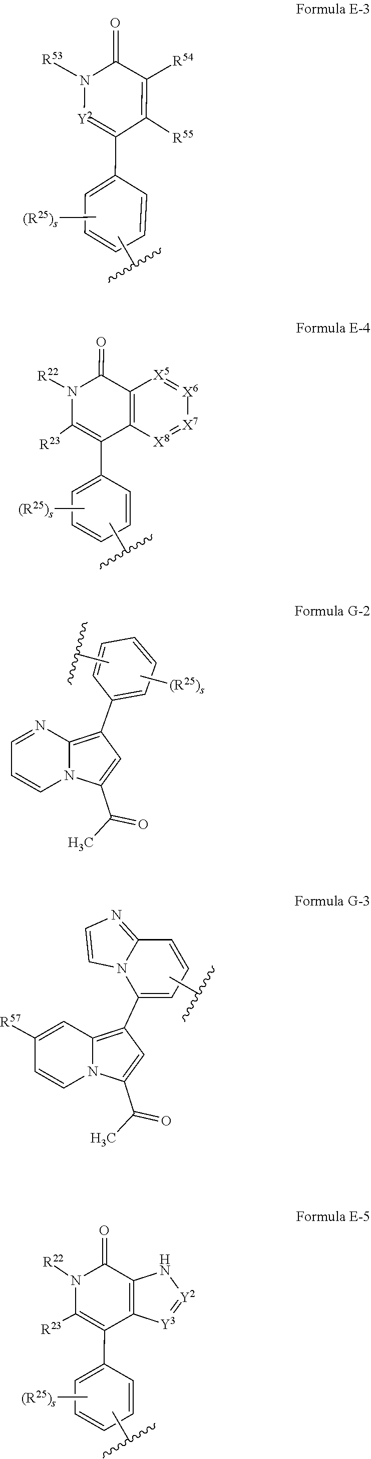
wherein
Y² is N or CR²³;
R²² is H, optionally substituted C₁-C₆ alkyl, or optionally substituted C₁-C₆ heteroalkyl;
R²³ is H, halogen, optionally substituted C₁-C₆ alkyl, or optionally substituted C₆-C₁₀ aryl;
s is 0, 1, 2, 3, or 4;
each R²⁵ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₅-C₁₀ aryl, optionally substituted C₂-C₉ heteroaryl, optionally substituted C₂-C₆ alkenyl, optionally substituted C₂-C₆ heteroalkenyl, hydroxyl, thiol, or optionally substituted amino;
R⁵³ is H, optionally substituted C₁-C₆ alkyl, optionally substituted C₁-C₆ heteroalkyl, or optionally substituted C₃-C₁₀ carbocyclyl;
R⁵⁴ is H or optionally substituted C₂-C₉ heteroaryl;
R⁵⁵ is H or N(R^{a})₂, wherein each R^{a} is independently H, optionally substituted C₁-C₆ alkyl, optionally substituted C₁-C₆ heteroalkyl, or optionally substituted C₃-C₁₀ carbocyclyl, or two geminal R^{a}, together with the nitrogen atom to which they are attached, combine to form optionally substituted C₂-C₉ heterocyclyl;
each of X⁵, X⁶, X⁷, and X⁸ is, independently, N or CR⁵⁶;
each R⁵⁶ is, independently, H or N(R^{a})₂, wherein R^{a} is H, optionally substituted C₁-C₆ alkyl, optionally substituted C₁-C₆ heteroalkyl, or optionally substituted C₃-C₁₀ carbocyclyl, or two geminal R^{a}, together with the nitrogen atom to which they are attached, combine to form optionally substituted C₂-C₉ heterocyclyl;
R⁵⁷ is optionally substituted C₂-C₁₀ heterocyclyl;
each of Y² and Y³ is, independently, N or CR⁵⁸; and
each R⁵⁸ is, independently, H or optionally substituted C₁-C₆ alkyl,
wherein if R⁵³ is H and R⁵⁴ is H, then R⁵⁵ is NR^{a}; if R⁵⁴ is H and R⁵⁵ is H, then R⁵³ is optionally substituted C₃-C₁₀ carbocyclyl; and if R⁵³ is H and R⁵⁵ is H, then R⁵⁴ is optionally substituted C₂-C₉ heteroaryl.

63. The method of claim 62, wherein A has the structure of Formula E-3.

64. The method of claim 62, wherein A has the structure of Formula E-4.

65. The method of claim 62, wherein A has the structure of Formula G-2.

66. The method of claim 62, wherein A has the structure of Formula G-3.

67. The method of claim 62, wherein A has the structure of Formula E-5.

68. The method of any one of claims 62 to 67, wherein s is 0, 1, or 2.

69. The method of any one of claims 51 to 68, wherein the linker has the structure of Formula II:
A¹-(B¹)_{f}-(C¹)_{g}-(B²)ₕ-(D)-(B³)ᵢ-(C²)ⱼ-(B⁴)ₖ-A² Formula II
wherein
A¹ is a bond between the linker and A;
A² is the valency of Z;
each of B¹, B², B³, and B⁴ is, independently, optionally substituted C₁-C₂ alkyl, optionally substituted C₁-C₃ heteroalkyl, optionally substituted C₂₋₉ heterocyclyl, O, S, S(O)₂, or NR^{N};
R^{N} is H, optionally substituted C₁₋₄ alkyl, optionally substituted C₂₋₄ alkenyl, optionally substituted C₂₋₄ alkynyl, optionally substituted C₂₋₉ heterocyclyl, optionally substituted C₆₋₁₂ aryl, or optionally substituted C₁₋₇ heteroalkyl;
each of C¹ and C² is, independently, carbonyl, thiocarbonyl, sulphonyl, or phosphoryl;
f, g, h, I, j, and k are each, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₉ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}-(C¹)_{g}-(B²)ₕ-to-(B³)ᵢ-(C²)ⱼ-(B⁴)ₖ-A².

70. The method of claim 69, wherein each of B¹, B², B³, and B⁴ is, independently, optionally substituted C₁-C₄ alkyl, optionally substituted C₁-C₄ heteroalkyl, or NR^{N}.

71. The method of claim 69 or 70, wherein R^{N} is H or optionally substituted C₁₋₄ alkyl.

72. The method of any one of claims 69 to 71, wherein R^{N} is H or CH₃.

73. The method of any one of claims 69 to 72, wherein each of B¹ and B⁴ is, independently,

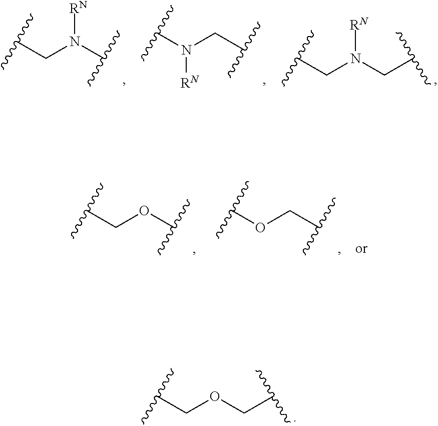

74. The method of claim 73, wherein B¹ is

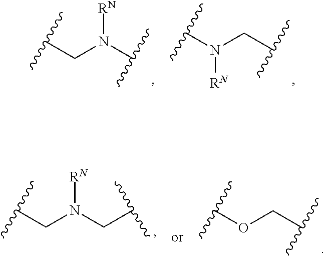

75. The method of any one of claims 69 to 74, wherein each of C¹ and C² is, independently,

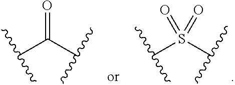

76. The method of claim 75, wherein C¹ is

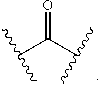 [SMILES: *C(*)=O]

77. The method of any one of claims 51 to 76, wherein the carboxybenzaldehyde is of the following structure:

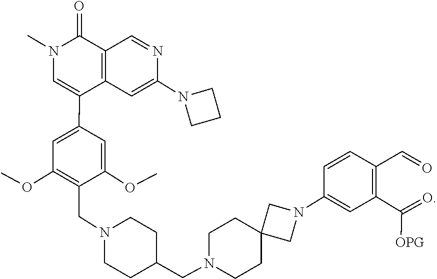

78. The method of claim 77, further comprising the step of preparing the carboxybenzaldehyde from a first reactant and a second reactant,
wherein the first reactant is of the following structure: 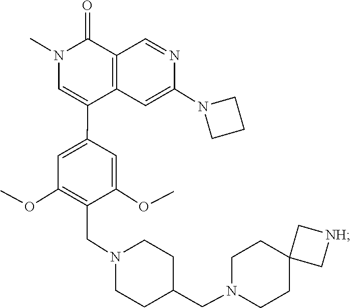 [SMILES: COc1cc(-c2cn(C)c(=O)c3cnc(N4CCC4)cc23)cc(OC)c1CN1CCC(CN2CCC3(CC2)CNC3)CC1]
and
wherein the second reactant is of the following structure:

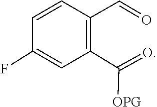

79. The method of claim 78, wherein the step of preparing the carboxybenzaldehyde is performed under the nucleophilic aromatic substitution reaction conditions.

80. The method of claim 78 or 79, further comprising the step of preparing the first reactant from a third reactant and a fourth reactant,
wherein the third reactant is a compound of the following structure: 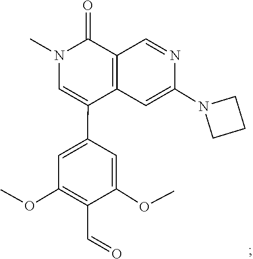 [SMILES: COc1cc(-c2cn(C)c(=O)c3cnc(N4CCC4)cc23)cc(OC)c1C=O]
and
wherein the fourth reactant is a compound of the following structure: 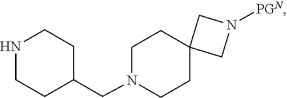
wherein PG^{N} is an N-protecting group.

81. The method of claim 80, wherein the step of preparing the first reactant comprises reacting the third reactant and the fourth reactant under reductive amination reaction conditions and removing the N-protecting group.

82. The method of any one of claims 51 to 81, further comprising the step of preparing the salt of the chiral glutarimide, wherein the step comprises reacting a free-base form of the chiral glutarimide with an acid to produce the salt of the chiral glutarimide.

83. The method of claim 82, wherein the acid is citric acid, and the salt of the chiral glutarimide is a citrate salt of the chiral glutarimide.
